(19) United States
(12) Patent Application Publication
(10) Pub. No.: US 2025/0382301 A1
(43) Pub. Date: Dec. 18, 2025

(54) SMALL MOLECULE INHIBITORS OF DYRK/CLK AND USES THEREOF

(71) Applicants: Arizona Board of Regents on Behalf of the University of Arizona, Tucson, AZ (US); University of Dundee, Dundee (GB)

(72) Inventors: Christopher Hulme, Tucson, AZ (US); Samantha Rokey, Tucson, AZ (US); Curtis Thorne, Tucson, AZ (US); Arthur Shaw, Tucson, AZ (US); Tim Chavez, Tucson, AZ (US); Nathan Bedard, Tucson, AZ (US); Carly Cabel, Tucson, AZ (US); Sourav Banerjee, Dundee (GB); Alessandra Fistrovich, Tucson, AZ (US)

(21) Appl. No.: 18/877,547

(22) PCT Filed: Jun. 22, 2023
(86) PCT No.: PCT/US2023/025973

§ 371 Date: Dec. 20, 2024

Related U.S. Application Data
(60) Provisional application No. 63/354,495, filed on Jun. 22, 2022.

(51) Int. Cl. C07D 487/08; A61K 31/496; A61K 31/517; A61K 31/519; A61K 31/551; C07D 401/14; C07D 403/14; C07D 405/14; C07D 417/14; C07D 471/04; C07D 471/10; C07D 487/04

(52) U.S. Cl. CPC C07D 487/08; A61K 31/496; A61K 31/517; A61K 31/519; A61K 31/551; C07D 401/14; C07D 403/14; C07D 405/14; C07D 417/14; C07D 471/04; C07D 471/10; C07D 487/04

(57) ABSTRACT

This invention is in the field of medicinal chemistry. In particular, the invention relates to a new class of small-molecule compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) which function as inhibitors of DYRK1A, DYRK1B, DYRK2, DYRK3, CLK1, CLK2, CLK3, CLK4, CDK7, CDK8/19, PI3K, PDGFrA/B, mTOR, WNT, homeodomain-interacting kinases (HIPKs), and/or CMGC kinases leading to inhibition of WNT signaling, and their use as therapeutics for the treatment of Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

### STATEMENT OF GOVERNMENT SUPPORT

This invention was made with government support under Grant No. AG067926 awarded by National Institutes of Health. The government has certain rights in the invention.

### FIELD OF THE INVENTION

This invention is in the field of medicinal chemistry. In particular, the invention relates to a new class of small-molecule compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) which function as inhibitors of DYRK1A, DYRK1B, DYRK2, DYRK3, CLK1, CLK2, CLK3, CLK4, CDK7, CDK8/19, PI3K, PDGFrA/B, mTOR, WNT, homeodomain-interacting kinases (HIPKs), and/or CMGC kinases leading to inhibition of WNT signaling, and their use as therapeutics for the treatment of Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

### INTRODUCTION

It's estimated that 2 million new cases of cancer will be shortly diagnosed per year with over 600,000 deaths in the US per annum. DYRK, CLK and CDK kinase inhibition affords opportunities across a spectrum of malignancies. Moreover, small molecule inhibition of DYRK and CLK kinases may play a role in mitigating disease progression of autoimmune disease and inflammatory disorders, exemplified by osteo-arthritis. DYRK1A has been revealed to play a key role in dementia and down syndrome pathogenesis. With >40 million patients suffering, dementia is currently a leading unmet medical need and costly burden on public health. Seventy percent of these cases have been attributed to Alzheimer's disease (AD), a neurodegenerative pathology whose most evident symptom is a progressive decline in cognitive functions. The underlying treatment of learning and/or memory disorders is a huge and significantly unmet medical need and also includes learning and memory repair after incidents of stroke or significant brain damage.

The present invention addresses these needs.

### SUMMARY OF THE INVENTION

The proteasome (immuno- and constitutive), heat shock factor 1, and mammalian target of rapamycin (mTOR) are essential protein complexes responsible for maintaining growth, division, and survival of cells in eukaryotes and are required for almost all cellular activities. Any impairment of any one or more of the complexes often underlies neurodegenerative diseases, cancer, immune disorders, and the aging process. Targeting these complexes have been clinically proven to be effective against all forms of cancers. RNA interference, kinome-wide screen, and biochemical studies demonstrate that blocking 26S proteasome and heat shock factor 1 phosphorylations triggered by DYRK2 markedly impairs proteostasis and impedes cell proliferation (see, Guo et. al. 2016 Nature Cell Biology; Moreno et. al. 2021 Cell death and differentiation; Banerjee et. al. PNAS 2018; Banerjee et. al. PNAS 2019). Furthermore, inhibition of DYRK3 activity leads to loss of PRAS40 phosphorylation leading to loss of mTOR signaling which reduces cancer cell proliferation. Importantly, loss of DYRK2 and DYRK3 activities significantly inhibited tumor formation in mice (see, eg, Banerjee et. al. 2019 PNAS). Accordingly, small-molecule inhibitors of DYRK kinases, either used alone or in combination with existing chemotherapy and/or proteasome inhibitors, have unique therapeutic potentials in treating human cancers with deregulated growth and proliferation.

Moreover, canonical WNT signaling is a key developmental pathway that has garnered significant interest for therapeutic intervention. The ability to modulate the WNT pathway and thus restore the health of diseased tissues affords possibilities in regenerative therapeutics and oncology. Noteworthy, WNT signaling controls chondrocyte osteoblast and synovial cell functions in osteoarthritis (Tao et. al., Theranostics, 2017, 7, 180-195). Indeed, numerous biological processes and targets related to WNT activation have been reported (Zhan et. al., Oncogene 2017, 36, 1461-1473. Ahmed et. al., Cancers, 2016, 8, 66). Part of this set of targets comprises the serine/threonine kinase CLKs shown to modulate the Wnt pathway by regulating pre-mRNA splicing (Deshmukh et. al., Osteoarthritis Cartilage 2019, 27, 1347-1360, Wang et. al., Nature 2008, 456-470-476). They consist as four isoforms in mammals (CLK1 through CLK4) and belong to the CMGC group of kinases which includes DYRKS, cyclin-dependent kinases (CDKs), GSK3, serine-arginine-rich protein kinases (SRPK) and others. CLK protein over-expression affects splicing site selection of pre-mRNA and as such, several CLK family inhibitors have been reported to play roles in the control mechanisms of mRNA splicing (Bossard et al., Cancer Res., 2020, 80, 5691. Deshmukh et. al., Osteoarthritis Cartilage 2019, 27, 1347-1360). Specifically, two high profile CLK inhibitors in clinical trials are SM08502 (Indication: Colorectal cancer, NCT03355066) and SM04690 (Indication: Osteoarthritis of the knee, Phase 3, NCT03928184).

CDKs have also shown to be heavily implicated in WNT inhibition. In particular CDK7 enhances the interaction between beta-catenin and TCF4 (see Duan et al., Cell Death & Differentiation, 2019, 26, 1442-1452), CDK8 has been identified as a gene that regulates b-catenin driven reporter activity in a loss of function RNAi screen (Rosenbluh et al., Trends Pharmacol Sci. 2014, 35, 103-109). Inhibitors of CDK7, 8 and 19 have shown utility in colorectal cancer and metastatic colon cancer in the liver and have been implicated in WNT signaling inhibition. Moreover, CDK8 selectivity promotes growth of colon cancer metastases in the liver by regulating gene expression of TIMP3 and matrix metalloproteases (see Liang et. al., Cancer Res. 2018, 78(23), 6594-6606). CDK8 and its paralog CDK19 are two isoforms of the Mediator kinase, the enzymatic component of the CDK module that binds to the transcriptional Mediator complex and inhibition of the CDK8/19 Mediator kinase sensitizes HER2+ breast cancers to HER2-targeting drugs preventing resistance in vitro and in vivo (see, e.g., Ding et al., PNAS, 2022, 119 (32), 1-11, e2201073119). High profile CDK8/CDK19 inhibitors in clinical trials include RVU120 in patients with Acute Myeloid Leukemia (AML) or high-risk Myelodysplastic Syndrome (HR-MDS) (NCT04021368), TSN084 (NCT05300438) and Senexin B, the first selective CDK8/19 inhibitor to enter clinical trials (NCT03065010).

In addition to the overwhelmingly prominent f-amyloid hypothesis being evaluated in a multitude of clinical trials through small molecule modulation of γ- and β-secretases and numerous immune-based approaches, aberrant phosphorylation of the tau protein is believed to significantly contribute to the development of AD and thus affords an alternate approach for therapeutic development. Tau is a cytoplasmic protein involved in the stabilization of microtubules under normal conditions. In AD, neuronal tau has been found to be excessively phosphorylated, with subsequent generation of aggregates of phosphorylated tau protein, known as “neurofibrillary tangles” (NFTs). NFTs and amyloid plaques are considered the most common hallmarks of AD and are correlated with neurofibrillary degeneration, neuronal death, and dementia.

Interestingly, several protein kinases have been implicated in neuronal development and, in particular, their overexpression and aberrant activation have been shown to play a significant role in the development of AD via tau phosphorylation. Dual specificity tyrosine phosphorylation regulated kinase-1A (DYRK1A) is important in neuronal development and plays a variety of functional roles within the adult central nervous system. The DYRK1A gene is located within the Down syndrome critical region (DSCR) on human chromosome 21 and current research suggests that overexpression of DYRK1A may be a significant factor leading to cognitive deficits in people with Alzheimer's disease (AD) and Down syndrome (DS).

Experiments conducted during the course of developing embodiments for the present invention designed, synthesized and biologically evaluated compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) as inhibitors of the dual specificity tyrosine phosphorylation regulated kinases (DYRKS) and CLKs, and their potential for use as therapeutics against WNT driven cancers and other disorders related to DYRK1A, DYRK1B, DYRK2, DYRK3, and CLK1, CLK2, CLK3 and CLK4 activities (e.g., DS, other neuropathology, cancer including glioblastoma, prostate cancer, metastatic breast cancer, metastatic lung cancer, multiple myeloma, secondary metastatic tumors of the brain, triple negative breast cancer, diabetes (T1D/T2D), cognitive enhancement). Many of such compounds are likely to exhibit activity against dual specificity tyrosine phosphorylation regulated kinase-1B (DYRK1B), dual specificity tyrosine phosphorylation regulated kinase-2 (DYRK2) (see, Tandon, et al., J. Biol. Chem 296 (2021)), dual specificity tyrosine phosphorylation regulated kinase-3 (DYRK3) (see, Kim, et al., Intl. J. Molecular Sciences 22, 2982 (2021)), and exhibit activity against other kinases implicated in a variety of disease states (e.g., dual specificity protein kinase CLK1 (Clk-1) and the cyclin-dependent kinases CDK7, CDK8 and CDK19.

The DYRK/CLK inhibitors described herein can also be considered as potential therapeutics for the treatment of developmental diseases such as Down syndrome, and neurodegenerative diseases such as Parkinson's disease, and Huntington's disease. Moreover, the DYRK inhibitors of the present invention have been also implicated as potential therapeutics for the treatment of glioblastomas and further potential utility is highlighted in the oncology arena (see, e.g., Ionescu et al., Mini-reviews in Medicinal Chemistry, 2012, 12, 1315-1329).

These novel DYRK/CLK inhibitors may also have utility as general cognitive enhancers, given the published findings that DYRK1A can phosphorylate sirtuin 1, a key regulator of learning and memory (see, e.g., Michan et al., J. Neurosci. 2010, 30(29), 9695-9707; Guo et al., J Biol. Chem. 2010, 285 (17), 13223-13232). Moreover, the effectiveness of small molecule inhibition of DYRK1A in mitigating both insoluble tau aggregates and amyloid plaques has been demonstrated (see, e.g., Branca et al., Aging Cell, 2017, 16(5), 1146-1154). The mechanistic rational for this was detailed previously (Smith et al., ACS Chem. Neuroscience, 2012, 3(11), 857-872). These novel DYRK/CLK inhibitors may also have further utility as results identify DYRK1A as a physiologically relevant regulator of T_{reg} cell differentiation and suggest a broader role for other DYRK family members in immune homeostasis. As such, new roles may be found in autoimmune diseases such as inflammatory bowel disease and type 1 diabetes (see, e.g., Khor B, et al., eLife 2015; 4:e05920).

Accordingly, this invention relates to a new class of small-molecule compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) which function as inhibitors of DYRK1A, DYRK1B, DYRK2, DYRK3, CLK1, CLK2, CLK3, CLK4, CDK7, CDK8/19, PI3K, PDGFrA/B, mTOR, WNT, homeodomain-interacting kinases (HIPKs), and/or CMGC kinases leading to inhibition of WNT signaling, and their use as therapeutics for the treatment of Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

In a particular embodiment, compounds encompassed within the following formulas are provided:

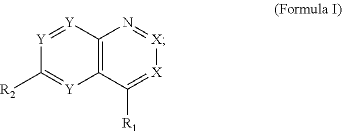

including pharmaceutically acceptable salts, solvates, and/or prodrugs thereof.

Formula I is not limited to a particular chemical moiety for X, Y. R1 and R2. In some embodiments, the particular chemical moiety for X, Y, R1 and R2 independently include any chemical moiety that permits the resulting compound to inhibit DYRK1A activity. In some embodiments, the particular chemical moiety for X, Y, R1 and R2 independently include any chemical moiety that permits the resulting compound to inhibit one or more of: DYRK1A related PI3K/Akt signaling; DYRK1A related tau phosphorylation; DYRK1A related NFAT phosphorylation; DYRK1A related ASK1/JNK1 pathway activation; DYRK1A related p53 phosphorylation; DYRK1A related Amph 1 phosphorylation; DYRK1A related Dynamin 1 phosphorylation; DYRK1A related Synaptojanin phosphorylation; DYRK1A related presenilin 1 (the catalytic sub-unit of γ-secretase) activity; DYRK1A related amyloid precursor protein phosphorylation; DYRK1A related SIRT1 activation; DYRK2 related heat shock factor 1 and 26S proteasome activities; DYRK3 related mTOR activity; DYRK3 phosphorylation (e.g., PRAS40); DYRK1B activity; CMGC/CLK kinase activity; CLK1 activity; CLK2 activity; CLK3 activity; CLK4 activity; CDK7 activity; CDK8 activity; CDK19 activity; PI3K activity; PI3K mutant activity; PDGFrA/B activity; mTOR activity; c-KIT activity; RYK activity; and WNT signaling.

Such embodiments are not limited to a particular definition for each of the “X” and “Y” substituents.

In some embodiments, one of the “X” substituents is carbon and the other is nitrogen, or both of the “X” substituents are carbon; and one of the “Y” substituents is nitrogen and the other “Y” substituents are carbon, or two of the “Y” substituents are nitrogen and one “Y” substituent is carbon, or all of the “Y” substituents are carbon: such that the resulting structure is one of the following formulas:

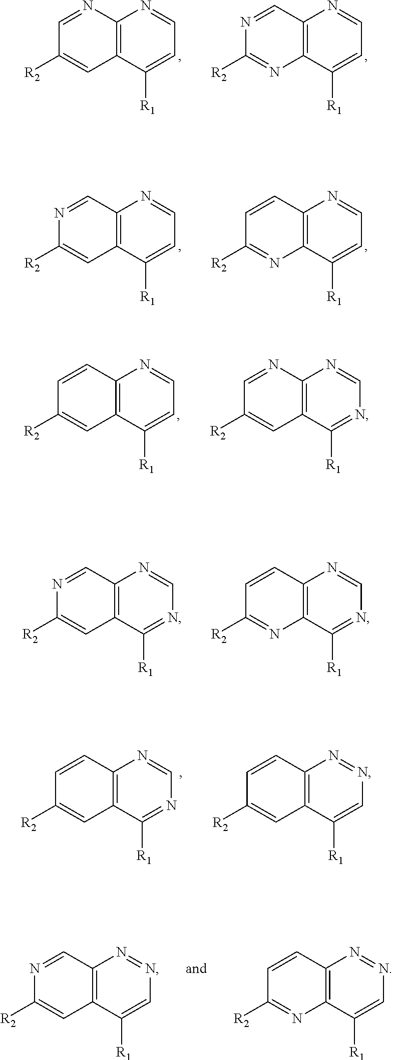

In some embodiments, R1 is selected from hydrogen,

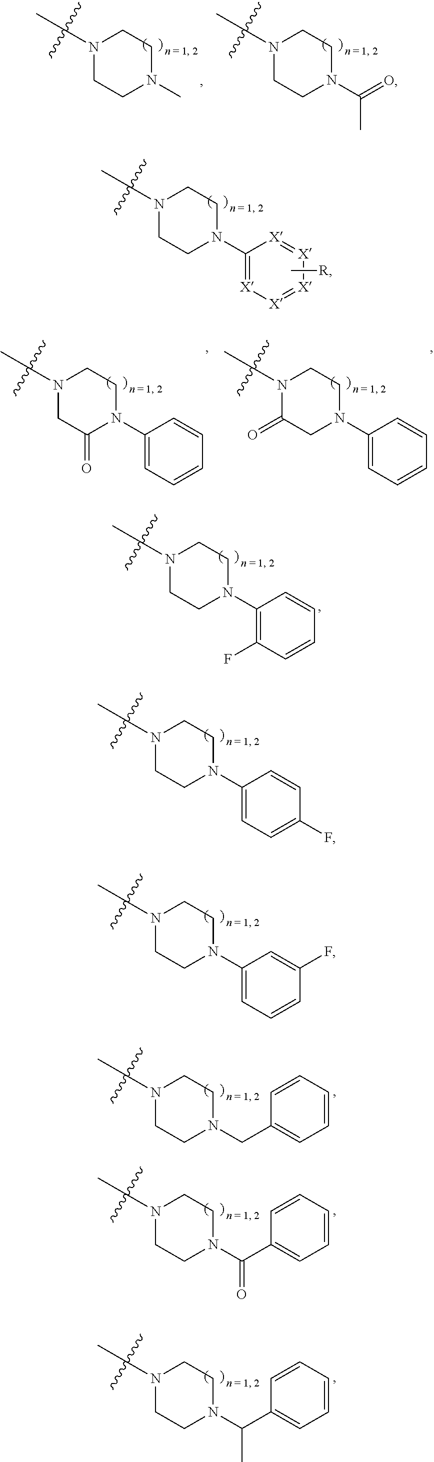

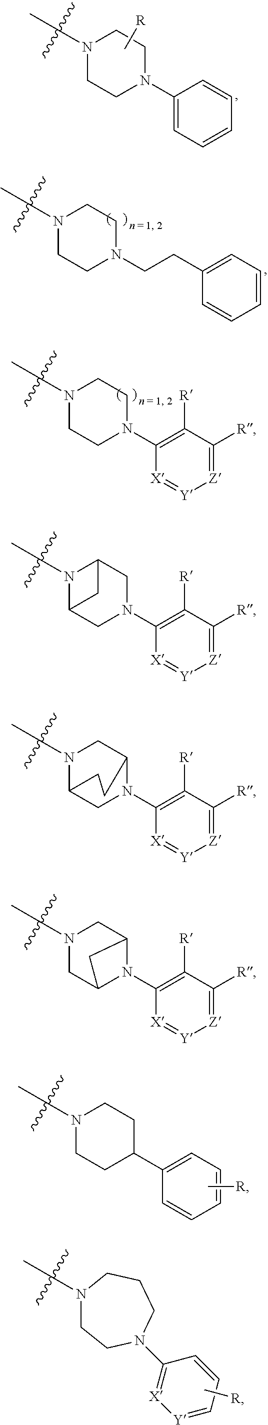

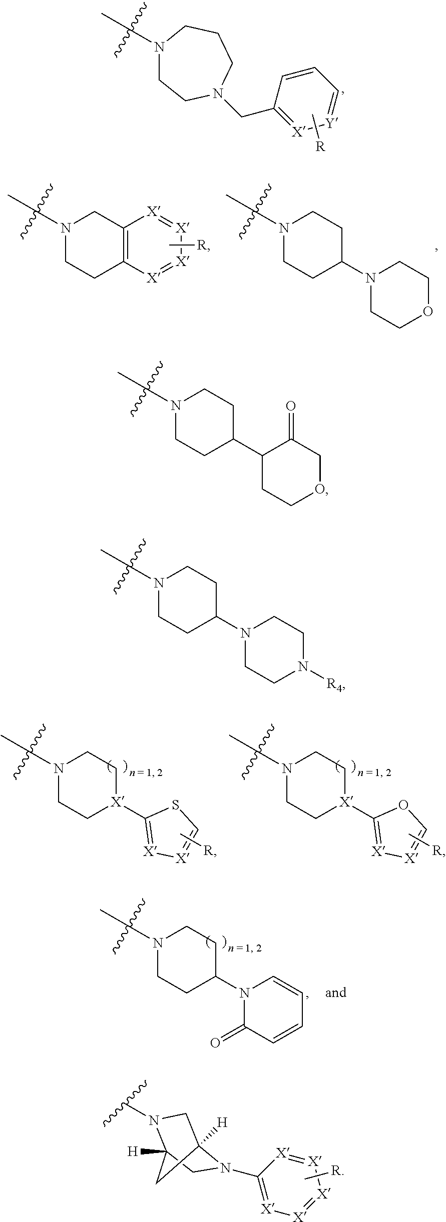

In some embodiments, R2 is selected from hydrogen, halogen (e.g., fluorine, bromine, iodine, chlorine), aryl, substituted aryl, heteroaryl, substituted heteroaryl,

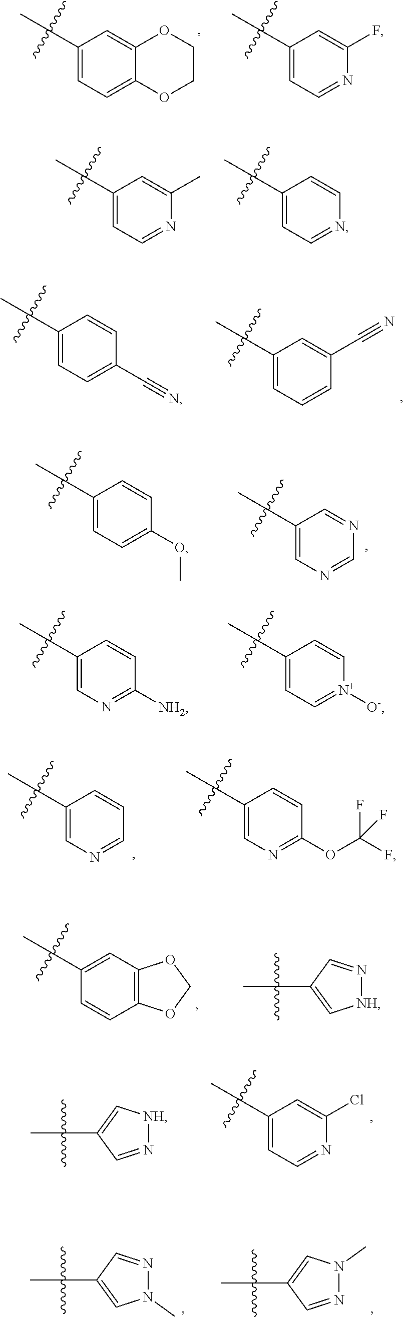

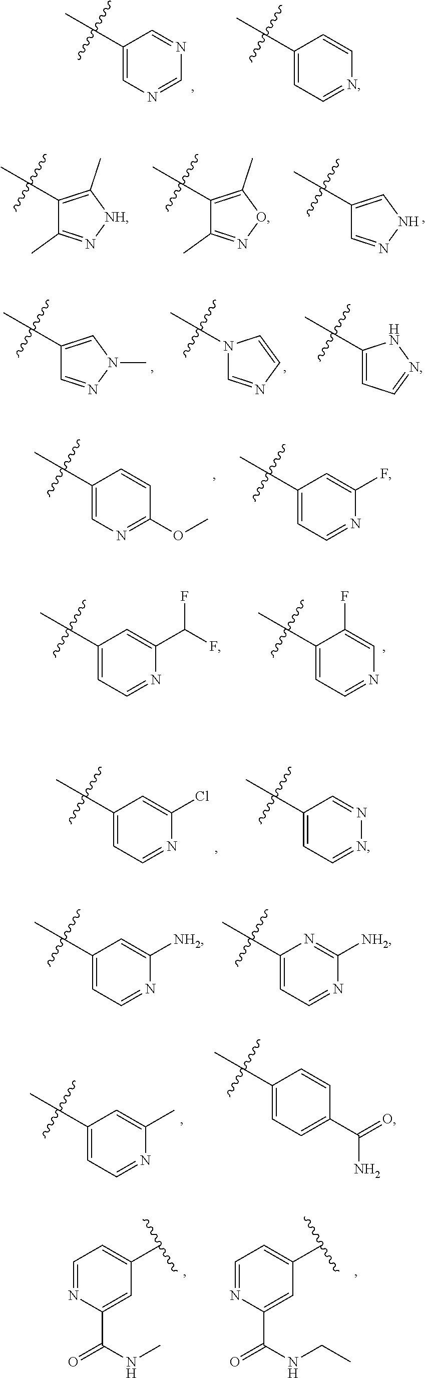

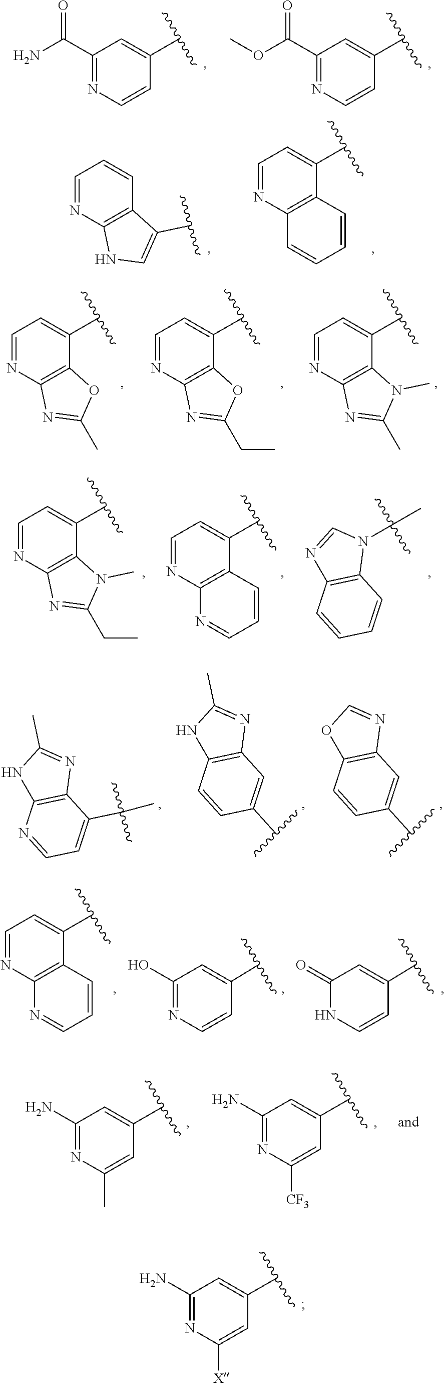

wherein X″ is selected from alkyl, haloalkyl, amino, alkylamino, hydroxy, fluoro, chloro, bromo, and cyano groups.

In some embodiments, X′, Y′, and Z′ are independently N, C or CR′.

In some embodiments, R, R′ and R″ are independently selected from hydrogen, halogen (e.g., fluorine, bromine, chlorine, iodine), di-halogen (di-fluorine, di-bromine, di-chlorine, di-iodine), CF3, OCH3, CHF2H, OCF3, methyl, di-methyl, alkoxy, alkylsulfonyl, cyano, carboxy, ester, amido, substituted amido, sulfonamide, substituted sulfonamide, methylenedioxy, heterocyclyl alkyl, heterocyclyl, heterocyclyl alkyl amido, a lipophilic moiety comprising ether, a secondary or tertiary amine moiety consisting of a heterocycloalkyl group that is bioisosteric to secondary amines (e.g., morpholine, piperidine, piperazine).

In some embodiments, R3 is selected from hydrogen, halogen (e.g., fluorine, bromine, chlorine, iodine), methyl, ethyl, and methoxy.

In some embodiments, R4 is selected from

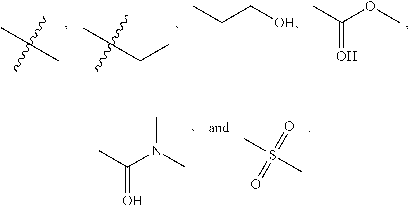

Each of the compounds presented in Table 1 have KD values between 0.5 nM to 10 uM (DYRK1A) and exhibit pan-DYRK and pan-CLK inhibitory profiles. Some of the compounds were shown to exhibit significant activity against CDK7, CDK8 and CDK19. Moreover, many of the exemplified compounds exhibit the ability to inhibit WNT signaling as judged by data from a WNT reporter assay—see Table 1. WNT Reporter Assay: Human Colonic Epithelial Cells (HCEC) were cultured using 1×DMEM supplemented with 1% penicillin/streptomycin, 1% Glutamax, and 10% fetal bovine serum in 5% CO2 at 37° C. These cells were previously engineered to express the TopGFP reporter (Addgene #24304) using second generation lentiviral techniques. For the Wnt reporter assay, cells were seeded at 2000 cells per well in a 384-well black screenstar imaging microplates (Greiner #781866) and allowed to adhere overnight. The following day cells were stimulated to induce the Wnt pathway using 10 μM CHIR99021 (Selleck #S1263). Simultaneously, DYR compounds were given in a dose-response using a Tecan d300e digital dispenser ranging from 0 μM to 30 μM concentrations. Cells were incubated for 24 hours before fixing for 30 minutes with 4% paraformaldehyde/sucrose solution. Cells were permeabilized with 0.1% triton-x in PBS for 10 minutes and stained for DAPI for 30 minutes. Plates were imaged on a Nikon Ti2 Eclipse fluorescent microscope for DAPI, GFP, and mCherry. Using Nikon Elements software for analysis, nuclei were segmented based on DAPI and mean object intensity per cell for both TopGFP and the internal control (mCherry) was measured. To calculate the amount of Wnt activity, we took the mean intensity of TopGFP and divided it by the mean intensity of mCherry per cell to normalize individual cells. Curves and EC50s were plotted and calculated using Graphpad Prism software.

TABLE 1

| | |
|---|---|
| 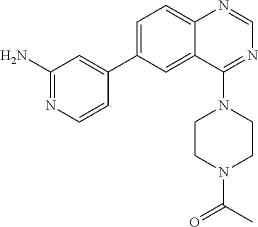 [SMILES: CC(=O)N1CCN(c2ncnc3ccc(-c4ccnc(N)c4)cc23)CC1] | ++ |
| 1 | |
| 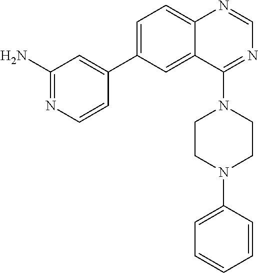 [SMILES: Nc1cc(-c2ccc3ncnc(N4CCN(c5ccccc5)CC4)c3c2)ccn1] | +++ |

| | |
|---|---|
| 2 | |
| 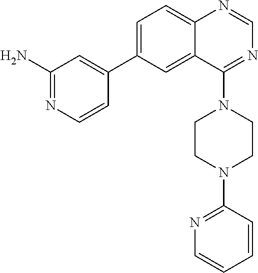 [SMILES: Nc1cc(-c2ccc3ncnc(N4CCN(c5ccccn5)CC4)c3c2)ccn1] | +++ |

| | |
|---|---|
| 3 | |
| 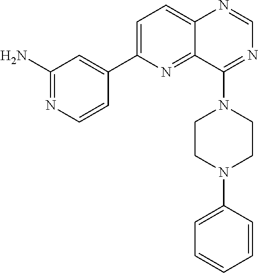 [SMILES: Nc1cc(-c2ccc3ncnc(N4CCN(c5ccccc5)CC4)c3n2)ccn1] | +++ |
| 4 | |
| 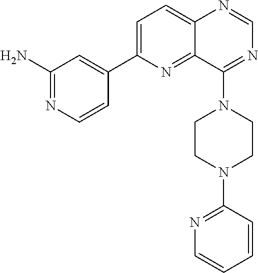 [SMILES: Nc1cc(-c2ccc3ncnc(N4CCN(c5ccccn5)CC4)c3n2)ccn1] | +++ |
| 5 | |
| 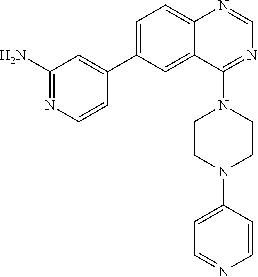 [SMILES: Nc1cc(-c2ccc3ncnc(N4CCN(c5ccncc5)CC4)c3c2)ccn1] | ++ |
| 6 | |
| 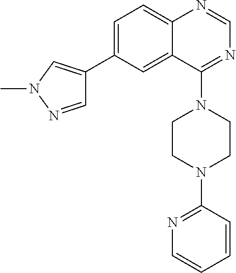 [SMILES: Cn1cc(-c2ccc3ncnc(N4CCN(c5ccccn5)CC4)c3c2)cn1] | ++ |
| 7 | |
| 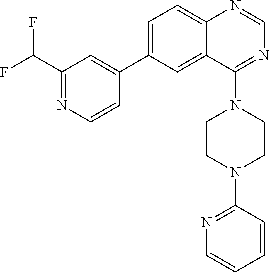 [SMILES: FC(F)c1cc(-c2ccc3ncnc(N4CCN(c5ccccn5)CC4)c3c2)ccn1] | ++ |
| 8 | |
| 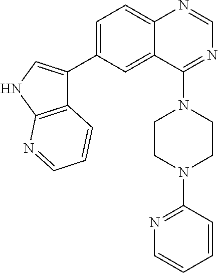 [SMILES: c1ccc(N2CCN(c3ncnc4ccc(-c5c[nH]c6ncccc56)cc34)CC2)nc1] | +++ |
| 17 | |
| 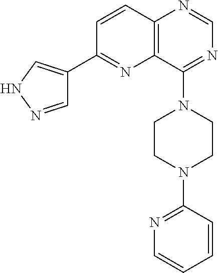 [SMILES: c1ccc(N2CCN(c3ncnc4ccc(-c5cn[nH]c5)nc34)CC2)nc1] | +++ |
| 18 | |
| 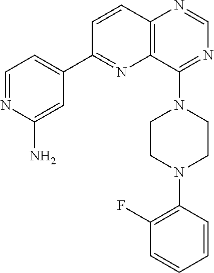 [SMILES: Nc1cc(-c2ccc3ncnc(N4CCN(c5ccccc5F)CC4)c3n2)ccn1] | +++ |
| 19 | |
| 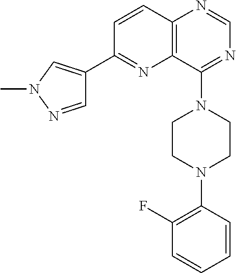 [SMILES: Cn1cc(-c2ccc3ncnc(N4CCN(c5ccccc5F)CC4)c3n2)cn1] | + |
| 20 | |
| 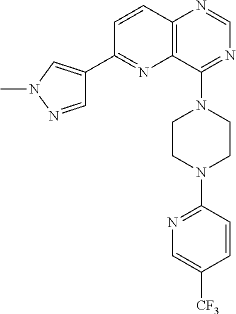 [SMILES: Cn1cc(-c2ccc3ncnc(N4CCN(c5ccc(C(F)(F)F)cn5)CC4)c3n2)cn1] | + |
| 21 | |
| 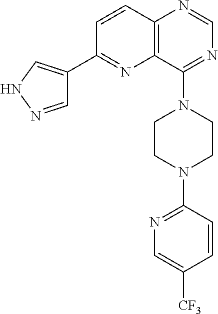 [SMILES: FC(F)(F)c1ccc(N2CCN(c3ncnc4ccc(-c5cn[nH]c5)nc34)CC2)nc1] | +++ |
| 22 | |
| 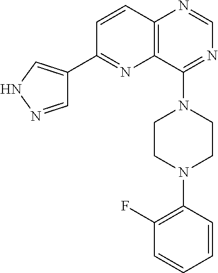 [SMILES: Fc1ccccc1N1CCN(c2ncnc3ccc(-c4cn[nH]c4)nc23)CC1] | +++ |
| 23 | |
| 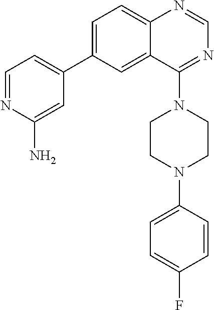 [SMILES: Nc1cc(-c2ccc3ncnc(N4CCN(c5ccc(F)cc5)CC4)c3c2)ccn1] | ++ |
| 31 | |
| 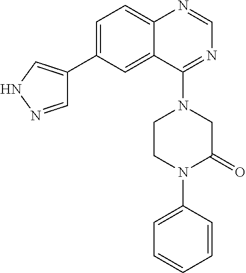 [SMILES: O=C1CN(c2ncnc3ccc(-c4cn[nH]c4)cc23)CCN1c1ccccc1] | +++ |
| 32 | |
| 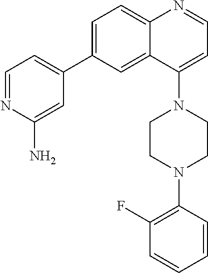 [SMILES: Nc1cc(-c2ccc3nccc(N4CCN(c5ccccc5F)CC4)c3c2)ccn1] | ++ |
| 33 | |
| 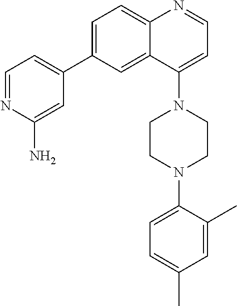 [SMILES: Cc1ccc(N2CCN(c3ccnc4ccc(-c5ccnc(N)c5)cc34)CC2)c(C)c1] | ++ |
| 34 | |
| 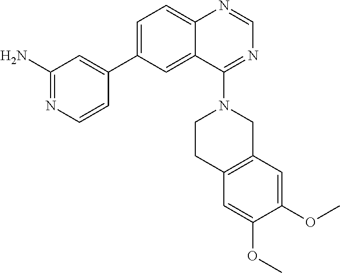 [SMILES: COc1cc2c(cc1OC)CN(c1ncnc3ccc(-c4ccnc(N)c4)cc13)CC2] | ++ |
| 35 | |
| 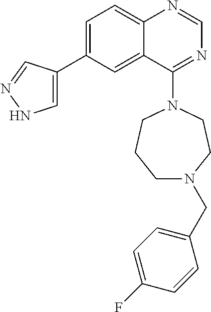 [SMILES: Fc1ccc(CN2CCCN(c3ncnc4ccc(-c5cn[nH]c5)cc34)CC2)cc1] | +++ |
| 36 | |
| 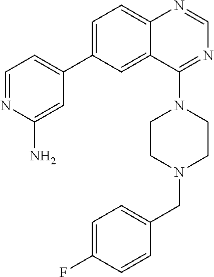 [SMILES: Nc1cc(-c2ccc3ncnc(N4CCN(Cc5ccc(F)cc5)CC4)c3c2)ccn1] | ++ |
| 43 | |
| 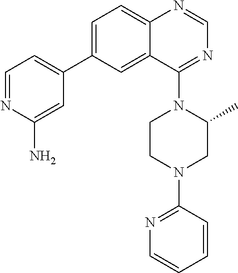 [SMILES: C[C@@H]1CN(c2ccccn2)CCN1c1ncnc2ccc(-c3ccnc(N)c3)cc12] | ++ |
| 44 | |
| 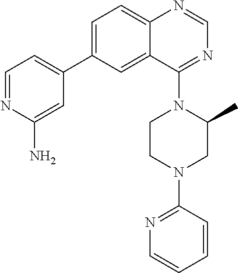 [SMILES: C[C@H]1CN(c2ccccn2)CCN1c1ncnc2ccc(-c3ccnc(N)c3)cc12] | +++ |
| 45 | |
| 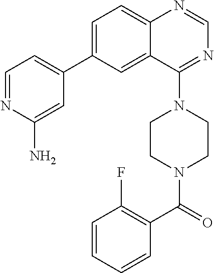 [SMILES: Nc1cc(-c2ccc3ncnc(N4CCN(C(=O)c5ccccc5F)CC4)c3c2)ccn1] | + |
| 46 | |
| 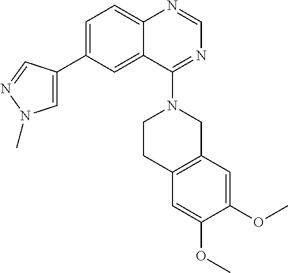 [SMILES: COc1cc2c(cc1OC)CN(c1ncnc3ccc(-c4cnn(C)c4)cc13)CC2] | + |
| 47 | |
| 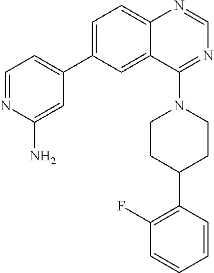 [SMILES: Nc1cc(-c2ccc3ncnc(N4CCC(c5ccccc5F)CC4)c3c2)ccn1] | ++ |
| 48 | |
| 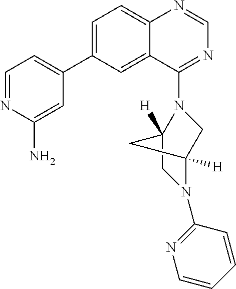 [SMILES: Nc1cc(-c2ccc3ncnc(N4C[C@@H]5C[C@H]4CN5c4ccccn4)c3c2)ccn1] | + |
| 55 | |
| 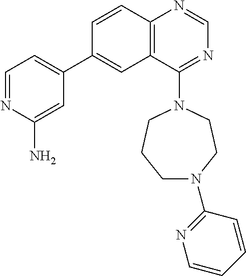 [SMILES: Nc1cc(-c2ccc3ncnc(N4CCCN(c5ccccn5)CC4)c3c2)ccn1] | + |
| 56 | |
| 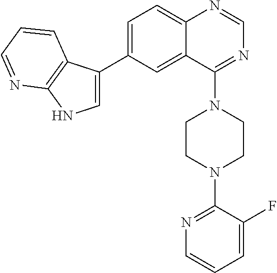 [SMILES: Fc1cccnc1N1CCN(c2ncnc3ccc(-c4c[nH]c5ncccc45)cc23)CC1] | +++ |
| 57 | |
| 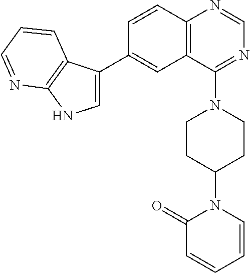 [SMILES: O=c1ccccn1C1CCN(c2ncnc3ccc(-c4c[nH]c5ncccc45)cc23)CC1] | +++ |
| 58 | |
| 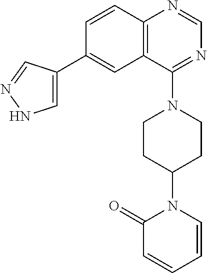 [SMILES: O=c1ccccn1C1CCN(c2ncnc3ccc(-c4cn[nH]c4)cc23)CC1] | +++ |
| 59 | |
| 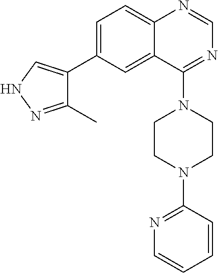 [SMILES: Cc1n[nH]cc1-c1ccc2ncnc(N3CCN(c4ccccn4)CC3)c2c1] | + |
| 60 | |
| 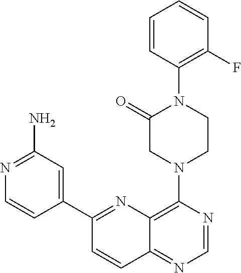 [SMILES: Nc1cc(-c2ccc3ncnc(N4CCN(c5ccccc5F)C(=O)C4)c3n2)ccn1] | +++ |
| 67 | |
| 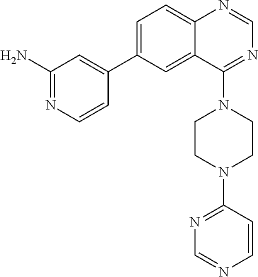 [SMILES: Nc1cc(-c2ccc3ncnc(N4CCN(c5ccncn5)CC4)c3c2)ccn1] | ++ |
| 9 | |
| 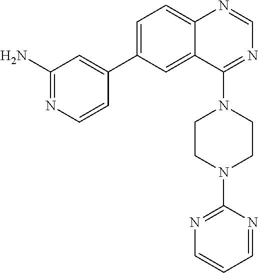 [SMILES: Nc1cc(-c2ccc3ncnc(N4CCN(c5ncccn5)CC4)c3c2)ccn1] | ++ |
| 10 | |
| 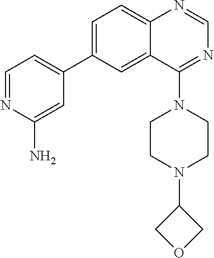 [SMILES: Nc1cc(-c2ccc3ncnc(N4CCN(C5COC5)CC4)c3c2)ccn1] | + |
| 11 | |
| 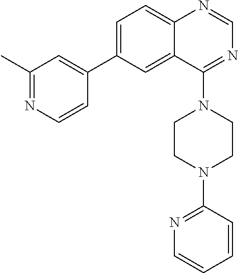 [SMILES: Cc1cc(-c2ccc3ncnc(N4CCN(c5ccccn5)CC4)c3c2)ccn1] | + |
| 12 | |
| 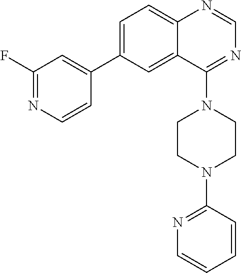 [SMILES: Fc1cc(-c2ccc3ncnc(N4CCN(c5ccccn5)CC4)c3c2)ccn1] | + |
| 13 | |
| 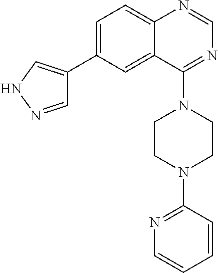 [SMILES: c1ccc(N2CCN(c3ncnc4ccc(-c5cn[nH]c5)cc34)CC2)nc1] | +++ |
| 14 | |
| 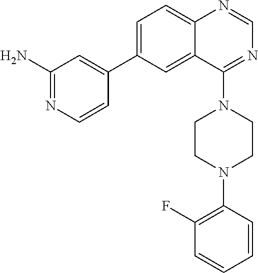 [SMILES: Nc1cc(-c2ccc3ncnc(N4CCN(c5ccccc5F)CC4)c3c2)ccn1] | ++ |
| 15 | |
| 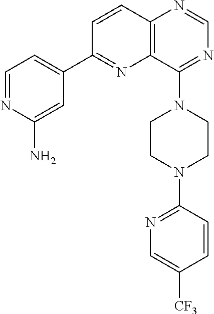 [SMILES: Nc1cc(-c2ccc3ncnc(N4CCN(c5ccc(C(F)(F)F)cn5)CC4)c3n2)ccn1] | +++ |
| 16 | |
| 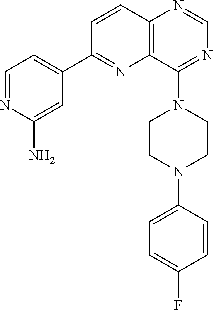 [SMILES: Nc1cc(-c2ccc3ncnc(N4CCN(c5ccc(F)cc5)CC4)c3n2)ccn1] | +++ |
| 24 | |
| 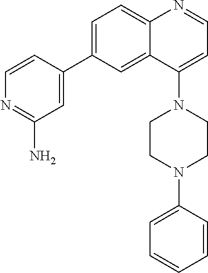 [SMILES: Nc1cc(-c2ccc3nccc(N4CCN(c5ccccc5)CC4)c3c2)ccn1] | +++ |
| 25 | |
| 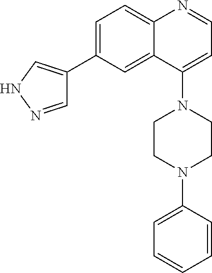 [SMILES: c1ccc(N2CCN(c3ccnc4ccc(-c5cn[nH]c5)cc34)CC2)cc1] | +++ |
| 26 | |
| 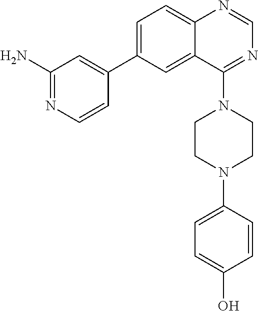 [SMILES: Nc1cc(-c2ccc3ncnc(N4CCN(c5ccc(O)cc5)CC4)c3c2)ccn1] | ++ |
| 27 | |
| 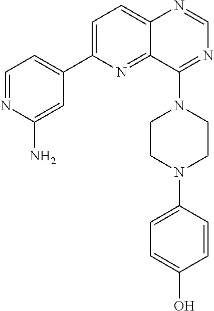 [SMILES: Nc1cc(-c2ccc3ncnc(N4CCN(c5ccc(O)cc5)CC4)c3n2)ccn1] | +++ |
| 28 | |
| 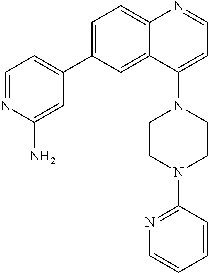 [SMILES: Nc1cc(-c2ccc3nccc(N4CCN(c5ccccn5)CC4)c3c2)ccn1] | +++ |
| 29 | |
| 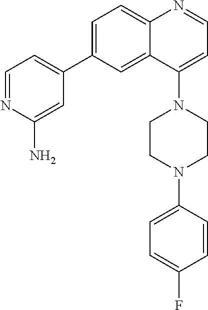 [SMILES: Nc1cc(-c2ccc3nccc(N4CCN(c5ccc(F)cc5)CC4)c3c2)ccn1] | +++ |
| 30 | |
| 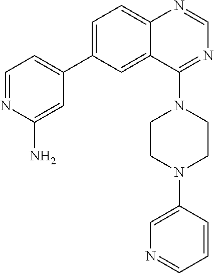 [SMILES: Nc1cc(-c2ccc3ncnc(N4CCN(c5cccnc5)CC4)c3c2)ccn1] | +++ |
| 37 | |
| 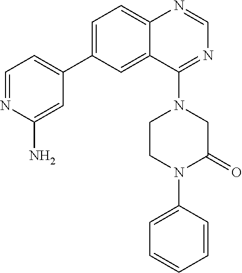 [SMILES: Nc1cc(-c2ccc3ncnc(N4CCN(c5ccccc5)C(=O)C4)c3c2)ccn1] | ++ |
| 38 | |
| 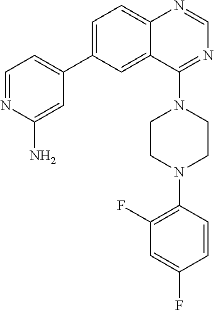 [SMILES: Nc1cc(-c2ccc3ncnc(N4CCN(c5ccc(F)cc5F)CC4)c3c2)ccn1] | ++ |
| 39 | |
| 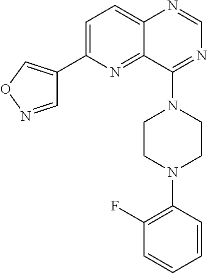 [SMILES: Fc1ccccc1N1CCN(c2ncnc3ccc(-c4cnoc4)nc23)CC1] | ++ |
| 40 | |
| 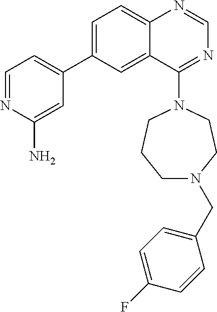 [SMILES: Nc1cc(-c2ccc3ncnc(N4CCCN(Cc5ccc(F)cc5)CC4)c3c2)ccn1] | ++ |
| 41 | |
| 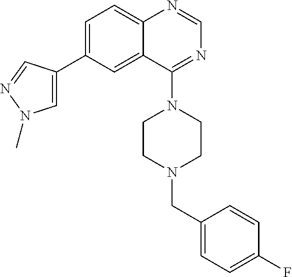 [SMILES: Cn1cc(-c2ccc3ncnc(N4CCN(Cc5ccc(F)cc5)CC4)c3c2)cn1] | + |
| 42 | |
| 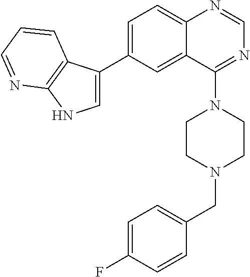 [SMILES: Fc1ccc(CN2CCN(c3ncnc4ccc(-c5c[nH]c6ncccc56)cc34)CC2)cc1] | +++ |
| 49 | |
| 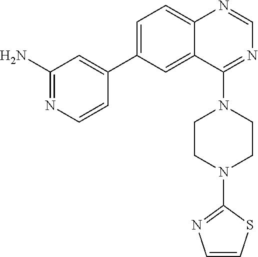 [SMILES: Nc1cc(-c2ccc3ncnc(N4CCN(c5nccs5)CC4)c3c2)ccn1] | +++ |
| 50 | |
| 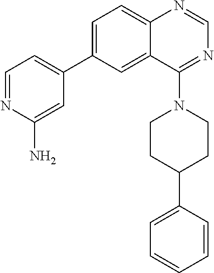 [SMILES: Nc1cc(-c2ccc3ncnc(N4CCC(c5ccccc5)CC4)c3c2)ccn1] | ++ |
| 51 | |
| 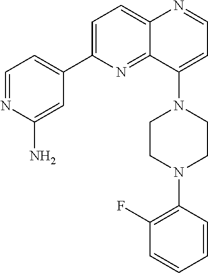 [SMILES: Nc1cc(-c2ccc3nccc(N4CCN(c5ccccc5F)CC4)c3n2)ccn1] | +++ |
| 52 | |
| 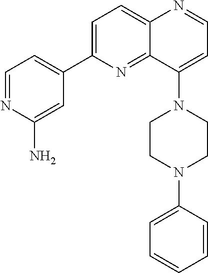 [SMILES: Nc1cc(-c2ccc3nccc(N4CCN(c5ccccc5)CC4)c3n2)ccn1] | +++ |
| 53 | |
| 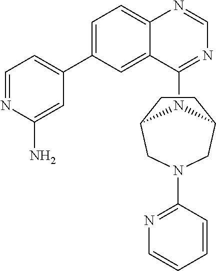 [SMILES: Nc1cc(-c2ccc3ncnc(N4[C@@H]5CC[C@H]4CN(c4ccccn4)C5)c3c2)ccn1] | ++ |
| 54 | |
| 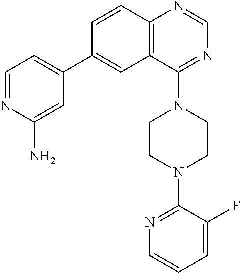 [SMILES: Nc1cc(-c2ccc3ncnc(N4CCN(c5ncccc5F)CC4)c3c2)ccn1] | +++ |
| 61 | |
| 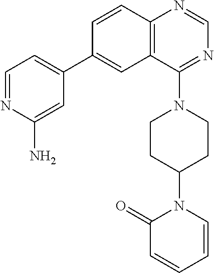 [SMILES: Nc1cc(-c2ccc3ncnc(N4CCC(n5ccccc5=O)CC4)c3c2)ccn1] | ++ |
| 62 | |
| 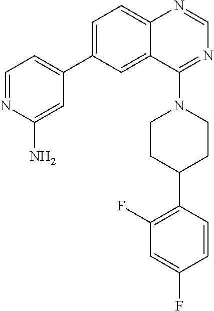 [SMILES: Nc1cc(-c2ccc3ncnc(N4CCC(c5ccc(F)cc5F)CC4)c3c2)ccn1] | ++ |
| 63 | |
| 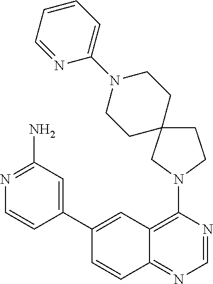 [SMILES: Nc1cc(-c2ccc3ncnc(N4CCC5(CCN(c6ccccn6)CC5)C4)c3c2)ccn1] | ++ |
| 64 | |
| 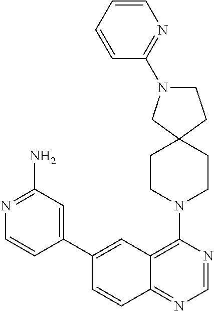 [SMILES: Nc1cc(-c2ccc3ncnc(N4CCC5(CCN(c6ccccn6)C5)CC4)c3c2)ccn1] | +++ |
| 65 | |
| 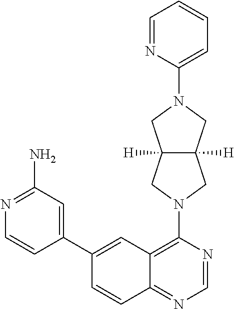 [SMILES: Nc1cc(-c2ccc3ncnc(N4C[C@H]5CN(c6ccccn6)C[C@H]5C4)c3c2)ccn1] | ++ |
| 66 | |

Activity Key in WNT reporter (EC50) +++ <100 nM, ++ 100 nM − 1 uM, >1 uM

The invention further provides processes for preparing any of the compounds of the present invention.

The invention also provides the use of compounds to not only inhibit DYRK1A activity but also signaling pathways dependent upon DYRK1A phosphorylation (e.g., Tau, PI3K/AKT, APP, PSI, ASF, RCAN-1, NEAT, p53, ASK1/JNK1, SIRT1, GluN2-A and other NMDA receptors), DYRK2 phosphorylation (e.g., 26S proteasome, heat shock factor 1, p53, MYC, and JUN), and DYRK3 phosphorylation (e.g., PRAS40). The invention also relates to the use of compounds for sensitizing cells to additional agent(s), such as agents known to be effective in the treatment of neurodegenerative disorders.

In certain embodiments, the compounds are used as DYRK protein degraders (see, Valazquez, et al, 2019 Molecular Neurobiology 1-12).

The compounds of the invention are useful for the treatment, amelioration, or prevention of disorders associated with DYRK1A, DYRK1B, DYRK2, DYRK3, CLK1, CLK2, CLK3, CLK4, homeodomain-interacting kinases (HIPKs), and/or CMGC kinases leading to inhibition of WNT signaling (e.g., Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases), such as those responsive to DYRK isoform activity inhibition. In certain embodiments, the compounds can be used to treat, ameliorate, or prevent cancer that is associated with DYRK2 and DYRK3 activities (e.g., glioblastoma, prostate cancer, metastatic breast cancer, metastatic lung cancer, multiple myeloma, secondary metastatic tumors of the brain, colorectal cancer). In certain embodiments, the compounds can be used to treat, ameliorate, or prevent autoimmune diseases. In certain embodiments, the compounds can be used to treat, ameliorate, or prevent inflammatory disorders (e.g., airway inflammation, osteoarthritis (e.g., knee related osteoarthritis)).

The invention also provides pharmaceutical compositions comprising the compounds of the invention in a pharmaceutically acceptable carrier.

The invention also provides kits comprising a compound of the invention and instructions for administering the compound to an animal. The kits may optionally contain other therapeutic agents, e.g., agents useful in treating neurodegenerative disorders and/or anticancer agents.

### DETAILED DESCRIPTION OF THE INVENTION

The DYRK family contains 5 kinases (DYRK1A, DYRK1B, DYRK2, DYRK3 and DYRK4). DYRKs belong to the CMGC group of proline-directed kinases, which also includes cyclin-dependent kinases (CDKs), mitogen-activated protein kinases (MAPKs), glycogen synthase kinases (GSKs) and CDC2-like kinases (CLKs). While the signaling pathways of CDK and MAPK families have been extensively studied, much less is known on how DYRKs and CLKs are linked to other proteins and various physiological or pathological processes. The CLK family comprises CLK1 through CLK4. The cyclin-dependent kinases (CDKs) are a family of multifunctional enzymes that can modify various protein substrates involved in cell cycle progression, comprising CDK1 through CDK19.

The DYRK1A gene is located on chromosome 21 (21q22.2), a region known as the Down-Syndrome Critical Region (DSCR) (see, e.g., Hammerle et al., 2011 Development 138, 2543-2554). The under- or over-expression of the Dyrkla gene in mammals or of its orthologous gene minibrain (mnb) in Drosophila causes severe retardation of central nervous system development and maturation. At the molecular level, DYRK1A phosphorylates the nuclear factor of activated T cells (NFAT), counteracting the effect of calcium signaling and maintaining inactive NFAT (see, e.g., Arron et al., 2006 Nature 411, 595-600). DYRK1A has been identified as a negative regulator of the cell cycle that promotes the switch to a quiescent state or differentiation (see, e.g., Chen et al., 2013 Mol. Cell 52, 87-100). In malignant cells, DYRK1A promotes survival via inhibition of pro-apoptotic proteins (see, e.g., Guo et al., 2010 J. Bio. Chem. 285, 13223-13232; Seifert et al., 2008 FEBS J. 275, 6268-6280).

Experiments conducted during the course of developing embodiments for the present invention designed, synthesized and biologically evaluated compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) as inhibitors of the dual specificity tyrosine phosphorylation regulated kinases (DYRKs: 1A, 1B, 2, 3, 4) and CLK family members (1, 2, 3, 4) for use as therapeutics against AD, down syndrome, multiple malignancies, in particular those associated with inhibition of WNT signaling and other disorders related to DYRK/CLK activity (e.g., DS, other neuropathology, glioblastoma, prostate cancer, metastatic breast cancer, metastatic lung cancer, multiple myeloma, secondary metastatic tumors of the brain). Note DYRKIB plays roles in survival of certain cancer cells and myoblast differentiation and has been validated as a promising target for CRPC.

Moreover, the DYRK/CLK inhibitors of the present invention can be used for treating other cellular pathways involved in mental impairment and neurodegenerative dementia. Specifically, the DYRK/CLK inhibitors of the present invention can be used for inhibiting DYRK1A activated PI3K/Akt signaling, a pathway largely involved in neuronal development, growth, and survival. The DYRK1A inhibitors of the present invention DYRK1A can be used for inhibiting DYRK1A stimulated ASK1/JNK1 activity, thereby inducing neuronal death and apoptosis. In addition, the DYRK1A inhibitors of the present invention DYRK1A can be used to inhibit DYRK1A phosphorylation of p53 during embryonic brain development, thereby preventing neuronal proliferation alteration. The DYRK1A inhibitors of the present invention can be used to inhibit DYRK1A phosphorylation of synaptic proteins Amph 1, Dynamin 1, and Synaptojanin, involved in the regulation of endocytosis, thereby retaining synaptic plasticity through preventing alteration of the number, size, and morphology of dendritic spines. The DYRK1A inhibitors of the present invention can be used to inhibit presenilin 1 (the catalytic sub-unit of γ-secretase). The DYRK1A inhibitors of the present invention can be used to inhibit DYRK2/3 & 4 activity. The DYRK1A inhibitors of the present invention can be used to inhibit DYRK1B activity. The DYRK1A inhibitors of the present invention can be used to inhibit CMGC CLK1-4 kinase activity.

As such, the present invention addresses the need for effective therapies for GBM, AD and DS by providing potent and pan-selective DYRK/CLK inhibitors able to permeate the blood-brain barrier (BBB) and elicit on-mechanism therapeutic responses in animal models. Disease states in the periphery include colorectal cancer, castration-resistant prostate cancer and malignancies associated with inhibition of WNT signaling.

Accordingly, the present invention relates to a new class of small-molecule compounds having a 6,6-heterocyclic structure (e.g., compounds having a naphthyridine, pyrido-pyridazine, pyrido-pyrazine, quinoline, pyrazino-pyridazine, pyrimido-pyrimidine, quinazoline, quinoxaline or cinnoline ring system) which function as inhibitors of DYRK1A, DYRK1B, DYRK2, DYRK3, CLK1, CLK2, CLK3, CLK4, CDK7, CDK8/19, PI3K, PDGFrA/B, mTOR, WNT, homeodomain-interacting kinases (HIPKs), and/or CMGC kinases leading to inhibition of WNT signaling, and their use as therapeutics for the treatment of Alzheimer's disease, down syndrome, Parkinson's disease, Huntington's disease, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

The CDC2-like kinase (CLK) family contains four isoforms which are important in regulating the function of the spliceosome complex (see, e.g., Fedorov et al, Chem Biol. 201 1; 18(1):67-76). This complex, comprised of small nuclear RNAs (snRNA) and a large number of associated proteins, regulates the splicing of pre-mRNAs to give mature protein-encoding mRNAs. CLK1 is known to regulate the activity of the spliceosome via phosphorylation of the constituent serine-arginine-rich (SR) proteins (see, e.g., Bullock et al, Structure. 2009; 17(3):352-62). By controlling the activity of the spliceosome in this way, many genes are able express more than one mRNA leading to diversity in the translated proteins. The alternative protein iso forms transcribed from the same gene will often have different activities and physiological functions. Deregulation of alternative splicing has been linked to cancer, where a number of cancer-related proteins are known to be alternatively spliced (see, e.g., Druillennec et al, J Nucleic Acids. 2012; 2012:639062). An example of an alternatively spliced protein in cancer is Cyclin Dl, important for the progression of cancer cells through the cell cycle (see, e.g., Wang et al, Cancer Res. 2008; 68(14):5628-38).

Alternative splicing regulated by CLK1 has also been described to play a role in neurodegenerative diseases, including Alzheimer's and Parkinson's, via phosphorylation of the SR proteins of the spliceosome (see, e.g., Jain et al, Curr Drug Targets. 2014; 15(5):539-50). In the case of Alzheimer's, CLK1 is known to regulate the alternative splicing of the microtubule-associated protein TAU leading to an imbalance between TAU iso forms which is sufficient to cause neurodegeneration and dementia (see, e.g., Liu et al, Mol Neurodegener. 2008; 3:8).

Cyclin-dependent kinases (CDKs) have been shown to be heavily implicated in WNT inhibition. In particular, CDK7 enhances the interaction between beta-catenin and TCF4 (see Duan et al., Cell Death & Differentiation, 2019, 26, 1442-1452). CDK8 has been identified as a gene that regulates b-catenin driven reporter activity in a loss of function RNAi screen (see Rosenbluh et al., Trends Pharmacol Sci. 2014, 35, 103-109). Moreover, CDK8 selectivity promotes growth of colon cancer metatheses in the liver by regulating gene expression of TIMP3 and matrix metalloproteases (see Liang et. al., Cancer Res. 2018, 78(23), 6594-6606). Indeed, CDK8 and its paralog CDK19 are two isoforms of the Mediator kinase, the enzymatic component of the CDK module that binds to the transcriptional Mediator complex and inhibition of the CDK8/19 Mediator kinase sensitizes HER2+ breast cancers to HER2-targeting drugs preventing resistance in vitro and in vivo (see, e.g., Ding et al., PNAS, 2022, 119 (32), 1-11, e2201073119). High profile CDK8/CDK19 inhibitors in clinical trials include RVU120 in patients with Acute Myeloid Leukemia (AML) or high-risk Myelodysplastic Syndrome (HR-MDS) (NCT04021368), TSNO84 (NCT05300438) and Senexin B, the first selective CDK8/19 inhibitor to enter clinical trials (NCT03065010).

In the treatment of both cancer and neurological disease, there is thus undoubtedly an urgent need for compounds which potently inhibit DYRK and CLK kinases and CDK7, 8 and 19 whilst not affecting other closely-related kinases. The compounds described herein address this need.

In a particular embodiment, compounds encompassed within the following formulas are provided:

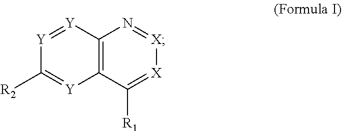

including pharmaceutically acceptable salts, solvates, and/or prodrugs thereof.

Formula I is not limited to a particular chemical moiety for X, Y, R1 and R2. In some embodiments, the particular chemical moiety for X, Y, R1 and R2 independently include any chemical moiety that permits the resulting compound to inhibit DYRK1A activity. In some embodiments, the particular chemical moiety for X, Y, R1 and R2 independently include any chemical moiety that permits the resulting compound to inhibit one or more of: DYRK1A related PI3K/Akt signaling; DYRK1A related tau phosphorylation; DYRK1A related NFAT phosphorylation; DYRK1A related ASK1/JNK1 pathway activation; DYRK1A related p53 phosphorylation; DYRK1A related Amph 1 phosphorylation; DYRK1A related Dynamin 1 phosphorylation; DYRK1A related Synaptojanin phosphorylation; DYRK1A related presenilin 1 (the catalytic sub-unit of γ-secretase) activity; DYRK1A related amyloid precursor protein phosphorylation; DYRK1A related SIRT1 activation; DYRK2 related heat shock factor 1 and 26S proteasome activities; DYRK3 related mTOR activity; DYRK3 phosphorylation (e.g., PRAS40); DYRK1B activity; CMGC/CLK kinase activity; CLK1 activity; CLK2 activity; CLK3 activity; CLK4 activity; CDK7 activity; CDK8 activity; CDK19 activity; PI3K activity; PI3K mutant activity; PDGFrA/B activity; mTOR activity; c-KIT activity; RYK activity; and WNT signaling.

Such embodiments are not limited to a particular definition for each of the “X” and “Y” substituents.

In some embodiments, one of the “X” substituents is carbon and the other is nitrogen, or both of the “X” substituents are carbon; and one of the “Y” substituents is nitrogen and the other “Y” substituents are carbon, or two of the “Y” substituents are nitrogen and one “Y” substituent is carbon, or all of the “Y” substituents are carbon; such that the resulting structure is one of the following formulas:

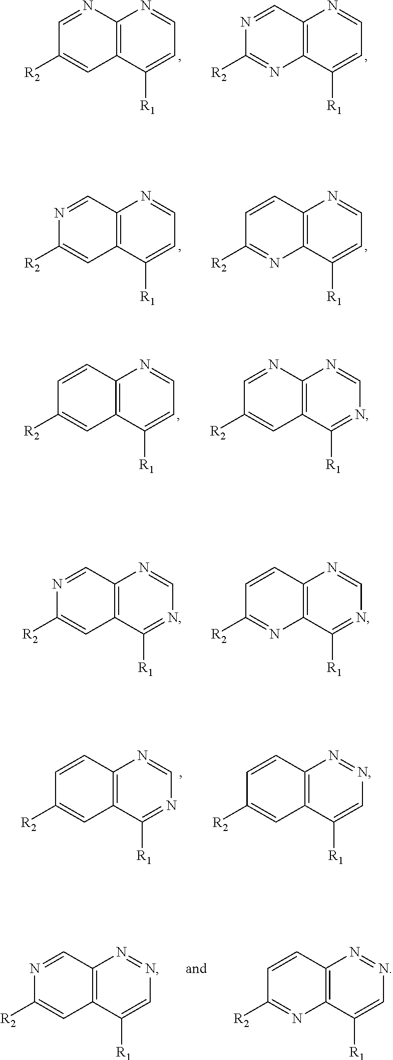

In some embodiments, R1 is selected from hydroen,

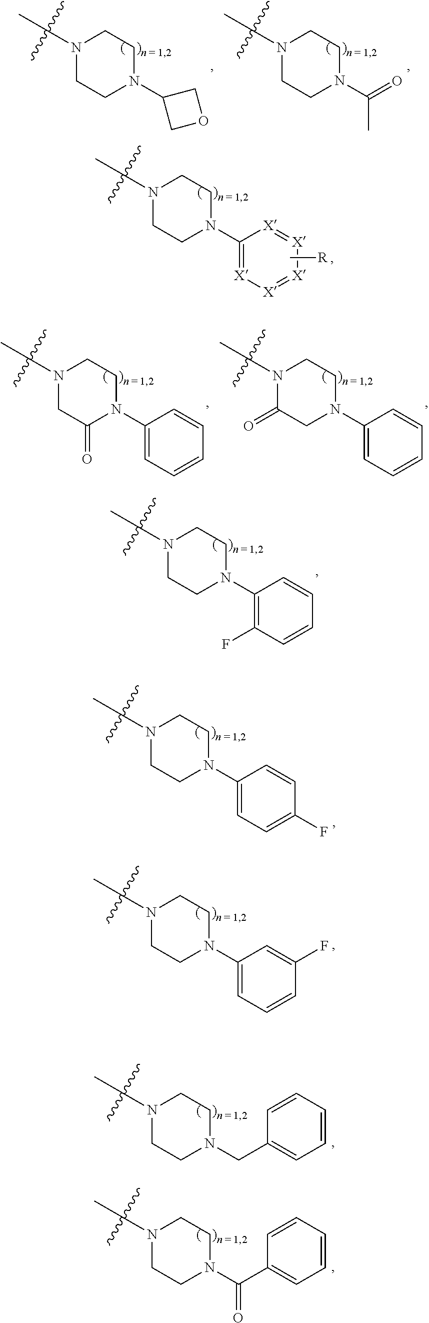

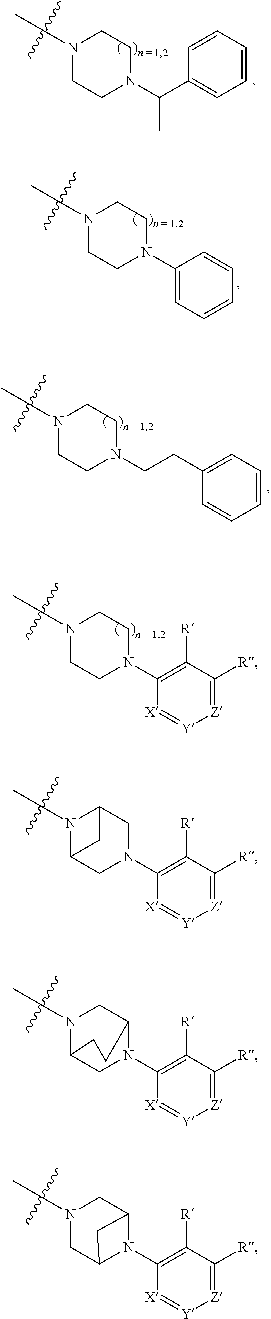

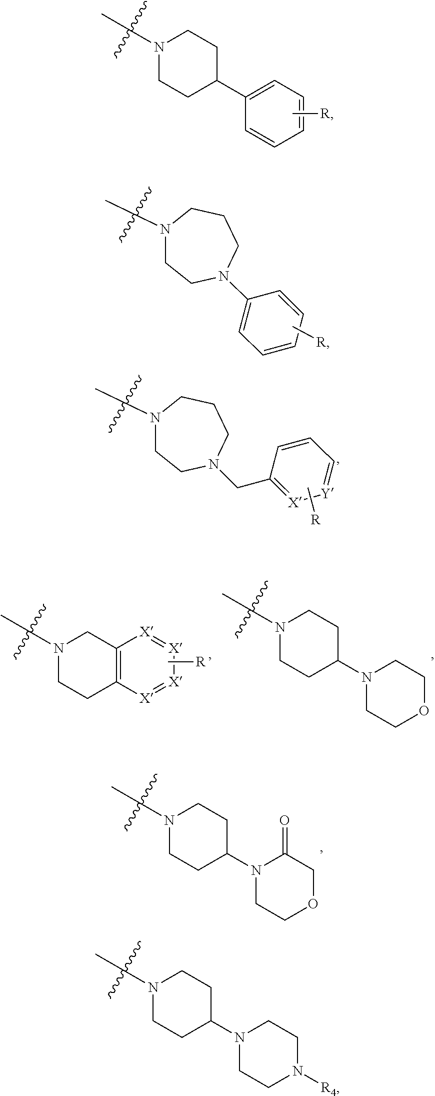

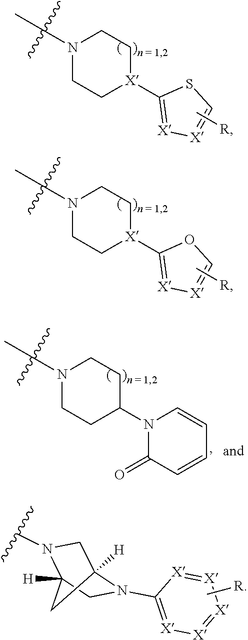

In some embodiments, R2 is selected from hydrogen, halogen (e.g., fluorine, bromine, iodine, chlorine), aryl, substituted aryl, heteroaryl, substituted heteroaryl,

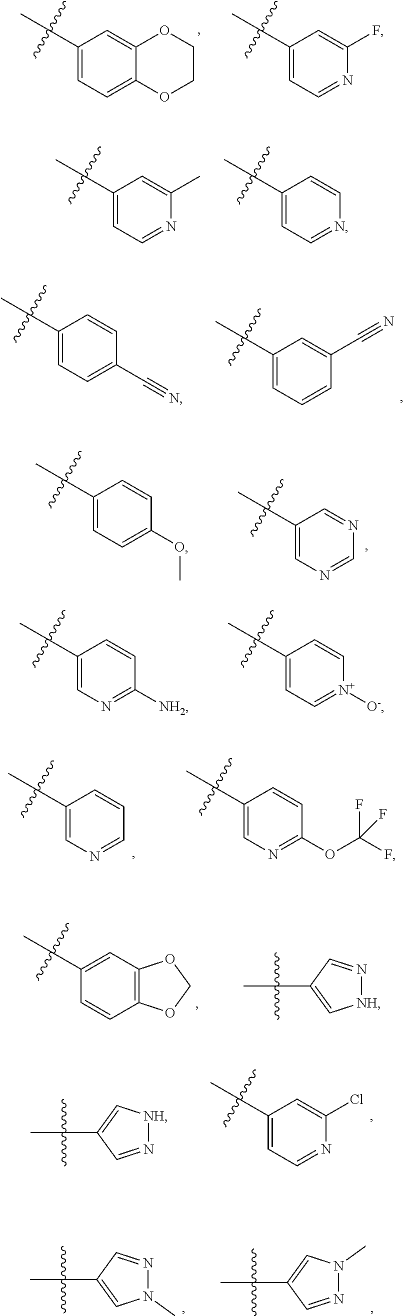

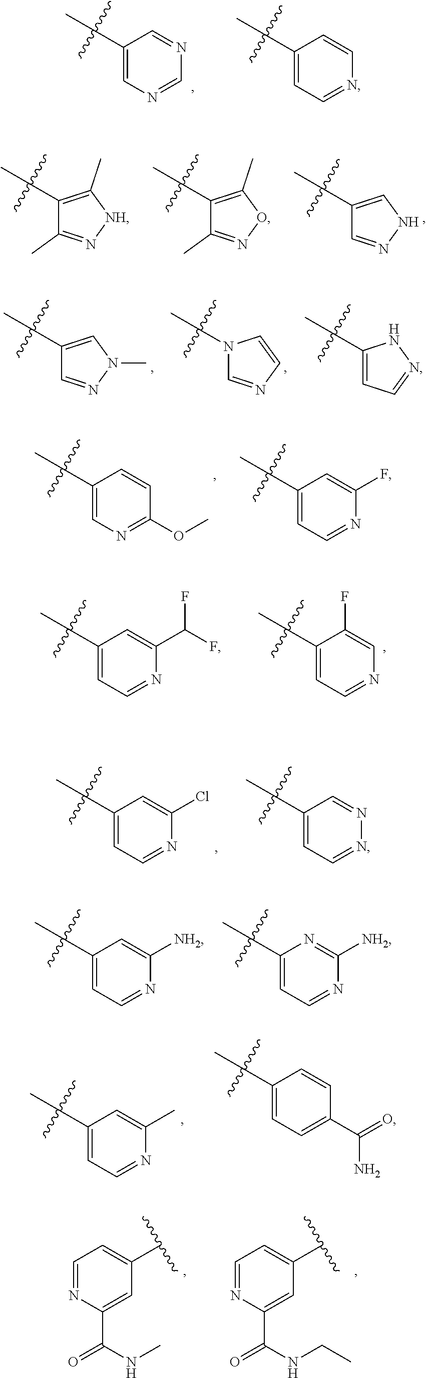

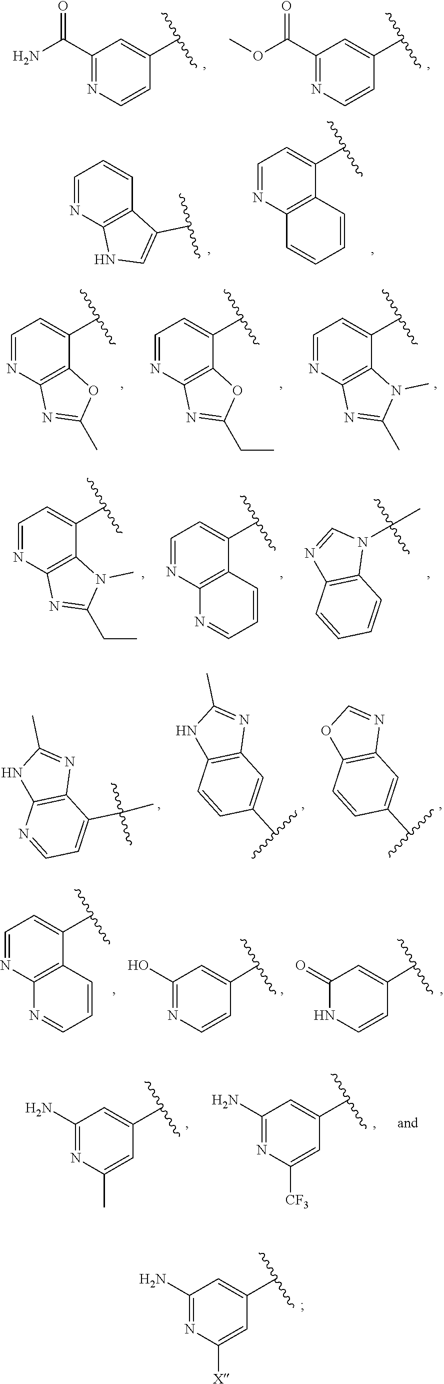

wherein X″ is selected from alkyl, haloalkyl, amino, alkylamino, hydroxy, fluoro, chloro, bromo, and cyano groups.

In some embodiments, X′, Y′, and Z′ are independently N, C or CR′.

In some embodiments, R, R′ and R″ are independently selected from hydrogen, halogen (e.g., fluorine, bromine, chlorine, iodine), di-halogen (di-fluorine, di-bromine, di-chlorine, di-iodine), CF3, OCH3, CHF2H, OCF3, methyl, di-methyl, alkoxy, alkylsulfonyl, cyano, carboxy, ester, amido, substituted amido, sulfonamide, substituted sulfonamide, methylenedioxy, heterocyclyl alkyl, heterocyclyl, heterocyclyl alkyl amido, a lipophilic moiety comprising ether, a secondary or tertiary amine moiety consisting of a heterocycloalkyl group that is bioisosteric to secondary amines (e.g., morpholine, piperidine, piperazine).

In some embodiments, R3 is selected from hydrogen, halogen (e.g., fluorine, bromine, chlorine, iodine), methyl, ethyl, and methoxy.

In some embodiments, R4 is selected from

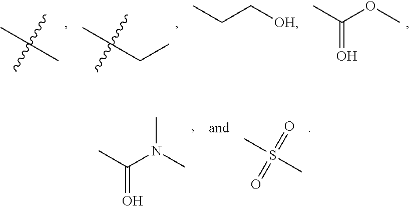

In some embodiments, the compounds are recited in Table 1. In some embodiments, the compound is one of Compounds 1-67 recited in Example I.

The invention further provides processes for preparing any of the compounds of the present invention.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, Alzheimer's disease, Down syndrome, Huntington's disease, Parkinson's disease, autoimmune diseases, cancer (e.g., glioblastoma, prostate cancer, metastatic breast cancer, metastatic lung cancer, multiple myeloma, secondary metastatic tumors of the brain), inflammatory disorders (e.g., airway inflammation), any neurodegenerative disorder related to DYRK1A, DYRK1B, DYRK2, DYRK3, CLK1, CLK2, CLK3, CLK4 activity, and/or CDK7, CDK8, CDK19 and any type of cancer related to DYRK1A, DYRK1B, DYRK2, DYRK3, CLK1, CLK2, CLK3, CLK4, and/or CDK7, CDK8, CDK19 activity, in particular those associated with disruption of WNT signaling.

Some embodiments of the present invention provide methods for administering an effective amount of a compound of the invention and at least one additional therapeutic agent (including, but not limited to, any agent useful in treating Alzheimer's disease, Down syndrome, Huntington's disease, Parkinson's disease, autoimmune diseases, inflammatory disorders (e.g., airway inflammation), any neurodegenerative disorder related to DYRK1A, DYRK1B, DYRK2, DYRK3, and/or CLK1, CLK2, CLK3 or CLK4 activity, in particular those associated with disruption of WNT signaling.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

This example provides synthesis and characterization information for compounds of the present invention.

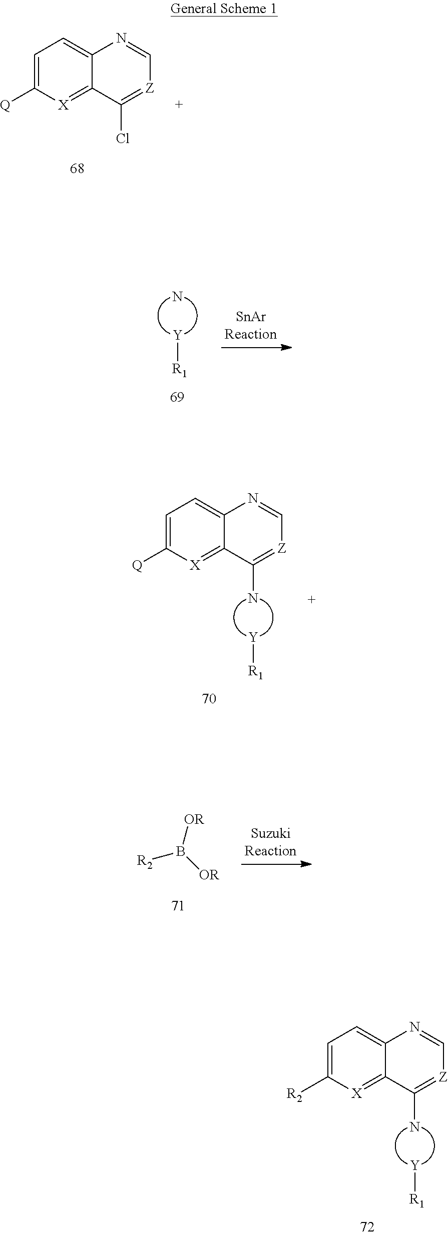

In General Scheme 1 68 is converted to 70 through standard SNAr chemistry by coupling with commercially available or synthetically prepared amines. 70 was then converted to 72 by Suzuki cross coupling chemistry by coupling with commercially available or synthetically prepared boronic acids and boronic esters. (X═CH or N, Q=Cl or Br, Z═CH or N, Y═N or CH).

### General Scheme 2

Four compounds (29, 30, 33, 34) were prepared by the following general route.

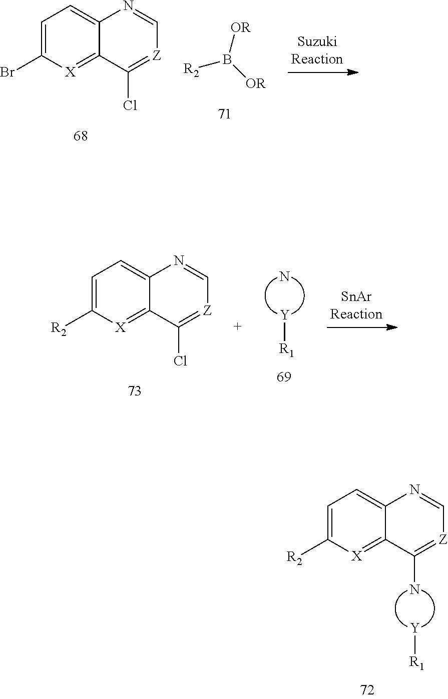

In General Scheme 1 68 is converted to 73 by Suzuki cross coupling with commercially available boronic acids and boronic esters. 73 was then converted to 72 through standard SNAr chemistry by coupling with commercially available or synthetically prepared secondary amines (X═CH or N, Z═CH or N, Y═N or CH).

### General Scheme 3

Two compounds (32, 67) were prepared by the following general route.

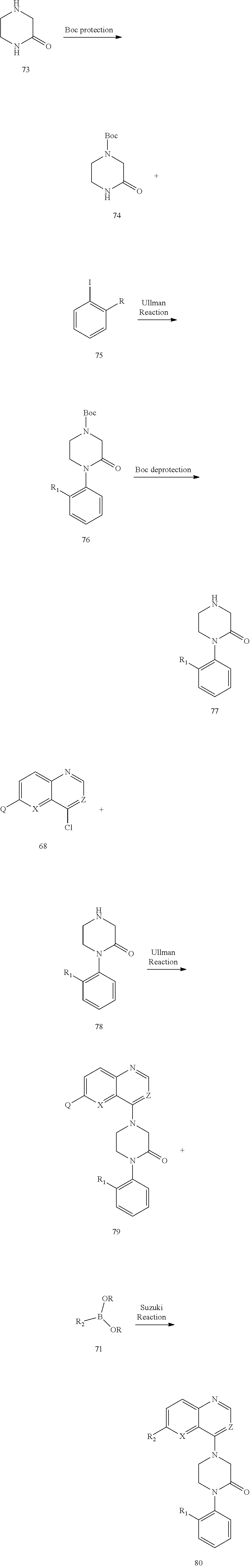

In General Scheme 3 73 is converted to 74 through a boc protection followed by an Ullmann coupling reaction with commercially available aryl halides. 76 was then converted to 77 by a Boc deprotection. A second Ullmann coupling reaction was employed to afford 79. 79 was then converted to 80 by Suzuki cross coupling chemistry by coupling with commercially available or synthetically prepared boronic acids and boronic esters. (X═CH or N, Q=Cl or Br, Z═CH or N, Y═N or CH).

### General Scheme 4

Two compounds (52, 53) were prepared by the following general route.

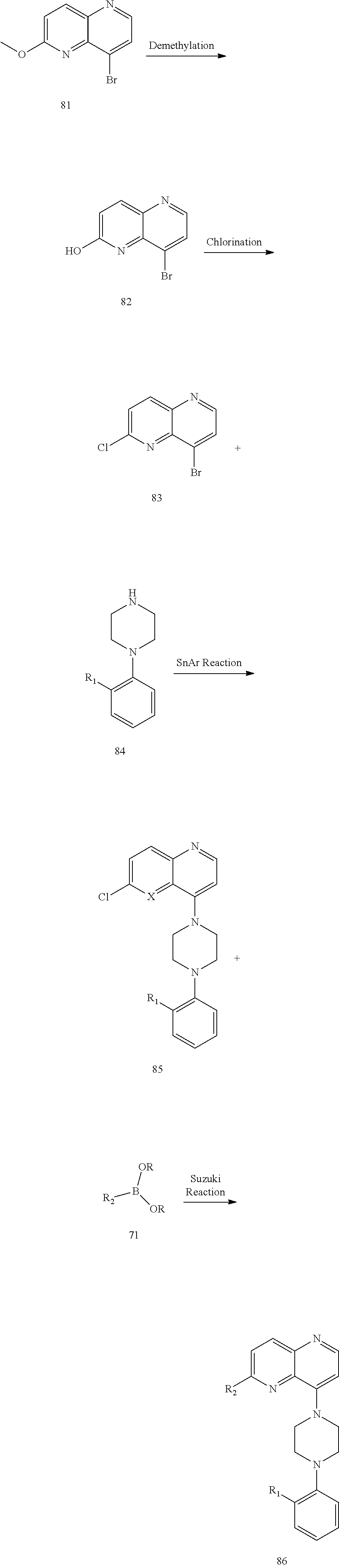

In General Scheme 4 81 is converted to 82 by demethylation followed by chlorination reaction to afford 83. Standard SnAr chemistry was employed to convert to 85 using commercially available or synthetically prepared secondary amines. 85 was then converted to 86 by Suzuki cross coupling chemistry by coupling with commercially available or synthetically prepared boronic acids and boronic esters.

### 2-(difluoromethyl)-4-(4,4,5,5-tetramethyl-1,3,2-dioxaborolan-2-yl)pyridine, 87

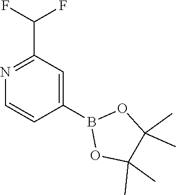 [SMILES: CC1(C)OB(c2ccnc(C(F)F)c2)OC1(C)C]

A 5 mL Biotage© microwave vial loaded with Bis(pinacolato)diborane (210 mg, 1.1 eq, 825 μmol), potassium acetate (147 mg, 2.0 eq, 1.50 mmol), [1,1′-Bis(diphenylphosphino)ferrocene]dichloropalladium(II) Complex with Dichloromethane (61 mg, 0.10 eq, 75.0 μmol), and 4-Bromo-alpha,alpha-difluoro-2-picoline (156 mg, 95 μL, 1 eq, 750 μmol). The vial was capped, purged with argon, then injected with degassed 1,4-Dioxane (5.0 mL, 0.14 M), and heated to 80° C. for 12 h. The reaction was cooled, concentrated, and the residue was taken up with sat NH4Cl, extracted with EtOAc and recrystallized with 75:25 Hexanes:DCM to afford 2-(difluoromethyl)-4-(4,4,5,5-tetramethyl-1,3,2-dioxaborolan-2-yl)pyridine, 87, as a white solid and used without further characterization.

### Step 1, General Scheme 1

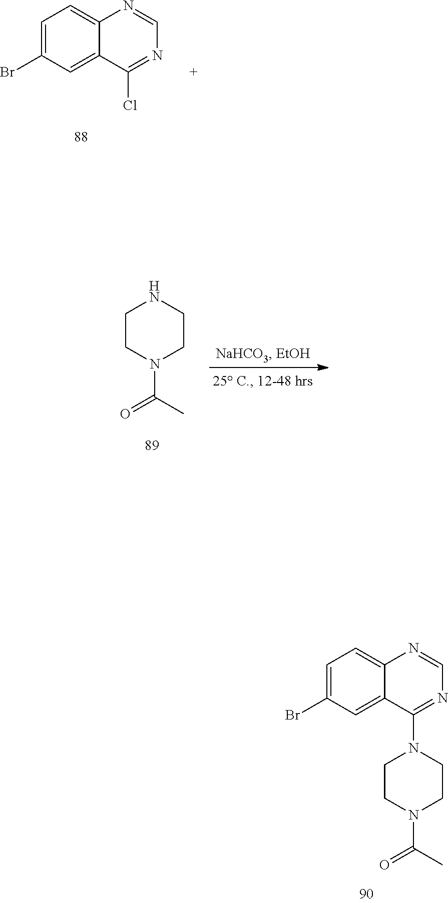

### 1-(4-(6-bromoquinazolin-4-yl)piperazin-1-yl)ethan-1-one, 90

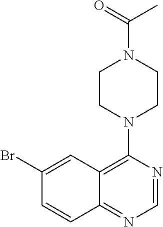 [SMILES: CC(=O)N1CCN(c2ncnc3ccc(Br)cc23)CC1]

In a 5 mL microwave vial, 6-bromo-4-chloroquinazoline (300 mg, 1 eq, 1.23 mmol), sodium bicarbonate (259 mg, 2.5 eq, 3.08 mmol), and 1-(piperazin-1-yl)ethan-1-one (205 mg, 1.3 eq, 1.60 mmol) were added. The mixture was capped, EtOH (1.2 mL) was injected, and stirred overnight at 25° C. for 48 hours. After this time, the mixture was poured into 20 mL H₂O and the vial washed with 5 mL H₂O×4 and filtered and washed with H₂O (5 mL×3), hexanes (5 mL×5), and dried over vacuum for 15 minutes affording 1-(4-(6-bromoquinazolin-4-yl)piperazin-1-yl)ethan-1-one, 90, (214 mg, 639 μmol, 52%). ¹HNMR (400 MHz, CDCl₃) δ 8.76 (s, 1H), 8.02 (dd, J=1.9, 0.7 Hz, 1H), 7.88-7.77 (m, 2H), 3.85-3.80 (m, 4H), 3.79-3.70 (m, 4H), 2.18 (s, 3H). ¹³CNMR (101 MHz, CDCl₃) δ 168.36, 163.90, 154.58, 150.20, 139.55, 132.16, 127.72, 121.01, 118.01, 55.60, 49.18, 45.75, 40.58, 23.92.

### Step 2, General Scheme 1

### 1-(4-(6-(2-aminopyridin-4-yl)quinazolin-4-yl)piperazin-1-yl)ethan-1-one, 1

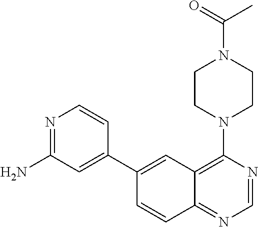 [SMILES: CC(=O)N1CCN(c2ncnc3ccc(-c4ccnc(N)c4)cc23)CC1]

A 5 mL microwave vial was loaded with 1-(4-(6-bromoquinazolin-4-yl)piperazin-1-yl)ethan-1-one (150 mg, 1 eq, 447 μmol), tert-butyl (4-(4,4,5,5-tetramethyl-1,3,2-dioxaborolan-2-yl)pyridin-2-yl)carbamate (158 mg, 1.1 eq, 492 μmol), PdCl₂(dppf) (35 mg, 0.1 eq, 44 μmol), and tripotassium phosphate (256 mg, 2.7 eq, 1.21 mmol), the vial capped then degassed for 10 minutes then injected with solvents degassed 1,4-dioxane (1.2 mL):water (0.30 mL), (4:1 v/v), degassed further for 10 min and heated to 130° C. for 60 min in an oil bath. The reaction was cooled and a vent needle was placed and cone HCl (2.20 g, 1.48 mL, 37% wt, 50 eq, 22.4 mmol) was added. The mixture was then stirred for 1 h and then 10% NaOH was added until basic. The mixture was diluted with H₂O and EtOAc and the aq. layer was extracted 3× with EtOAc. The pooled EtOAc extracts were washed 2× with brine and then the EtOAc layer was evaporated under vacuum and re-concentrated with DCM, dry-loaded onto silica gel and purified on a 12 g silica gel column (DCM/MeOH, 0-30%), affording 1-(4-(6-(2-aminopyridin-4-yl)quinazolin-4-yl)piperazin-1-yl)ethan-1-one, 1, (58 mg, 0.17 mmol, 37%) as a brown powder, which eluted at (0-10% DCM/MeOH). Mp 82-86° C. LCMS [M+H]⁺ 349. ¹HNMR (400 MHz, CDCl₃) δ 8.79 (s, 2H), 8.18 (d, J=5.4 Hz, 2H), 8.06 (d, J=1.7 Hz, 2H), 8.03-7.94 (m, 4H), 6.94-6.90 (m, 2H), 6.77 (s, 2H), 4.76 (s, 3H), 3.88 (dd, J=6.8, 4.0 Hz, 4H), 3.85 (s, 3H), 3.74 (dd, J=6.6, 3.7 Hz, 4H). ¹³CNMR (100 MHz, CDCl₃) δ 21.42, 41.19, 45.81, 49.10, 50.14, 106.34, 112.46, 116.62, 122.83, 129.64, 131.48, 136.19, 148.66, 149.23, 152.02, 154.41, 159.04, 164.68, 169.38.

### 4-(4-(4-phenylpiperazin-1-yl)quinazolin-6-yl)pyridin-2-amine, 2

Compound 2 was prepared via General Scheme 1 via the two step procedure reported for the preparation of compound 1. The two steps are reported below.

### 6-bromo-4-(4-phenylpiperazin-1-yl)quinazoline, 91

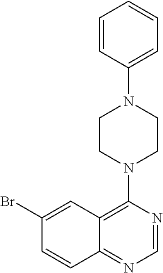 [SMILES: Brc1ccc2ncnc(N3CCN(c4ccccc4)CC3)c2c1]

In a 5 mL microwave vial, 6-bromo-4-chloroquinazoline (200 mg, 1 eq, 821 mol), sodium bicarbonate (173 mg, 2.5 eq, 2.05 mmol), and 1-phenylpiperazine (150 mg, 141 μL, 1.13 eq., 925 μmol) were added. The mixture was capped, iPrOH (1.2 mL) was injected, and stirred overnight at 25° C. for 3 hours. After this time, the mixture was poured into 20 mL H₂O and the vial washed with H₂O (5 mL×4) and filtered and washed with H₂O (5 mL×3), hexanes (5 mL×5), and dried over vacuum for 15 minutes affording 6-bromo-4-(4-phenylpiperazin-1-yl)quinazoline, 91, (247 mg, 669 μmol, 81%) as a white solid. δ 8.78 (s, 1H), 8.10 (d, J=1.4 Hz, 1H), 7.86 (d, J=1.3 Hz, 2H), 7.39-7.30 (m, 2H), 7.01 (d, J=8.1 Hz, 2H), 6.95 (t, J=7.3 Hz, 1H), 4.00 (t, J=5.1 Hz, 4H), 3.45 (dd, J=6.3, 3.9 Hz, 4H). ¹³CNMR (100 MHz, CDCl₃) δ 163.47, 156.12, 154.00, 150.84, 143.99, 136.12, 130.27, 129.31, 127.26, 120.46, 118.87, 117.63, 116.34, 49.70, 49.18.

### 4-(4-(4-phenylpiperazin-1-yl)quinazolin-6-yl)pyridin-2-amine, 2

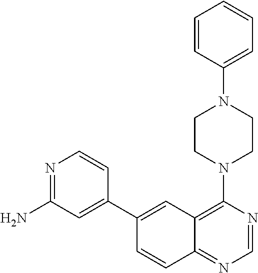 [SMILES: Nc1cc(-c2ccc3ncnc(N4CCN(c5ccccc5)CC4)c3c2)ccn1]

In a 5 mL microwave vial, 6-bromo-4-(4-(pyridin-2-yl)piperazin-1-yl)quinazoline (100 mg, 1 eq, 0.27 mmol), tert-butyl (4-(4,4,5,5-tetramethyl-1,3,2-dioxaborolan-2-yl)pyridin-2-yl)carbamate (95 mg, 1.10 eq, 0.297 mmol), tripotassium phosphate (83 mg, 1.5 eq, 0.41 mmol), and PdCl₂(dppf) (19 mg, 0.1 eq, 27 μmol) were added. The mixture was capped, dioxane (0.9 mL) was injected, and stirred at 130° C. for 30 min. After this time, the mixture was poured into 20 mL H₂O and the vial washed with H₂O (5 mL×4) and filtered and washed with H₂O (5 mL×3), hexanes (5 mL×5), and dried over vacuum for 15 minutes affording 4-(4-(4-phenylpiperazin-1-yl)quinazolin-6-yl)pyridin-2-amine, 2, as a brown solid. M.p. 94-100° C. LCMS [M+H]⁺ 383. ¹HNMR (400 MHz, CDCl₃) δ 8.78 (s, 1H), 8.10 (d, J=1.4 Hz, 1H), 7.86 (d, J=1.3 Hz, 2H), 7.39-7.30 (m, 2H), 7.01 (d, J=8.1 Hz, 2H), 6.95 (t, J=7.3 Hz, 1H), 4.00 (t, J=5.1 Hz, 4H), 3.45 (dd, J=6.3, 3.9 Hz, 4H). ¹³CNMR (101 MHz, CDCl₃) δ 163.47, 156.12, 154.00, 150.84, 143.99, 136.12, 130.27, 129.31, 127.26, 120.46, 118.87, 117.63, 116.34, 49.70, 49.18.

### 4-(4-(4-(pyridin-2-yl)piperazin-1-yl)quinazolin-6-yl)pyridin-2-amine, 3

Compound 3 was prepared via General Scheme 1 via the two step procedure reported for the preparation of compound 1. The two steps are reported below.

### 6-bromo-4-(4-(pyridin-2-yl)piperazin-1-yl)quinazoline, 92

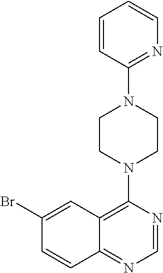 [SMILES: Brc1ccc2ncnc(N3CCN(c4ccccn4)CC3)c2c1]

In a 5 mL microwave vial, 6-bromo-4-chloroquinazoline (200 mg, 1 eq, 0.821 mmol), and 1-(pyridin-2-yl)piperazine (146 mg, 0.136 mL, 1.09 eq, 893 μmol) were added. The mixture was capped, EtOH (1.2 mL) was injected, and stirred at 25° C. for 48 hours. After this time, the mixture was poured into 20 mL H₂O and the vial washed with H₂O (5 mL×4) and filtered and washed with H₂O (5 mL×3), hexanes (5 mL×5), and dried over vacuum for 15 minutes affording 6-bromo-4-(4-(pyridin-2-yl)piperazin-1-yl)quinazoline, 92, (258 mg, 697 μmol, 85%) as a brown powder. Mp 100-105° C. ¹HNMR (400 MHz, CDCl₃) δ 8.80 (s, 1H), 8.25 (dd, J=5.0, 1.8 Hz, 1H), 8.17 (dd, J=16.8, 3.6 Hz, 2H), 8.03-7.95 (m, 2H), 7.56 (ddd, J=8.8, 7.1, 2.0 Hz, 1H), 6.95 (d, J=5.1 Hz, 1H), 6.79 (s, 1H), 6.75-6.68 (m, 2H), 4.79 (s, 1H), 4.00 (dd, J=6.7, 3.7 Hz, 4H), 3.84 (dd, J=6.5, 3.7 Hz, 4H). ¹³CNMR (101 MHz, CDCl₃) δ 164.74, 159.15, 154.53, 152.04, 149.45, 149.20, 148.50, 148.01, 137.70, 135.80, 131.29, 129.51, 123.15, 116.68, 113.94, 112.54, 107.20, 106.37, 49.49, 45.02.

### 4-(4-(4-(pyridin-2-yl)piperazin-1-yl)quinazolin-6-yl)pyridin-2-amine, 3

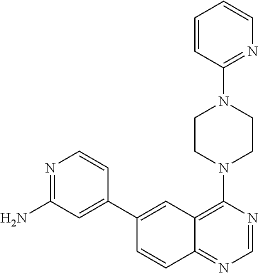 [SMILES: Nc1cc(-c2ccc3ncnc(N4CCN(c5ccccn5)CC4)c3c2)ccn1]

In a 5 mL microwave vial, 6-bromo-4-(4-(pyridin-2-yl)piperazin-1-yl)quinazoline (100 mg, 1 eq, 0.270 mmol), tert-butyl (4-(4,4,5,5-tetramethyl-1,3,2-dioxaborolan-2-yl)pyridin-2-yl)carbamate (95 mg, 1.10 eq, 0.297 mmol), tripotassium phosphate (83 mg, 1.5 eq, 0.41 mmol), and PdCl₂(dppf) (19 mg, 0.1 eq, 27.0 μmol) were added. The mixture was capped, dioxane (0.9 mL) was injected, and stirred at 130° C. for 30 min. After this time, the mixture was poured into 20 mL H₂O and the vial washed with H₂O (5 mL×4) and filtered and washed with H₂O (5 mL×3), hexanes (5 mL×5), and dried over vacuum for 15 minutes affording 4-(4-(4-(pyridin-2-yl)piperazin-1-yl)quinazolin-6-yl)pyridin-2-amine, 3, as a brown solid. Mp 100-105° C. LCMS [M+H]⁺ 384. ¹HNMR (400 MHz, CDCl₃) δ 8.80 (s, 1H), 8.25 (dd, J=5.0, 1.8 Hz, 1H), 8.17 (dd, J=16.8, 3.6 Hz, 2H), 8.03-7.95 (m, 2H), 7.56 (ddd, J=8.8, 7.1, 2.0 Hz, 1H), 6.95 (d, J=5.1 Hz, 1H), 6.79 (s, 1H), 6.75-6.68 (m, 2H), 4.79 (s, 1H), 4.00 (dd, J=6.7, 3.7 Hz, 4H), 3.84 (dd, J=6.5, 3.7 Hz, 4H). ¹³CNMR (101 MHz, CDCl₃) δ 164.74, 159.15, 154.53, 152.04, 149.45, 149.20, 148.50, 148.01, 137.70, 135.80, 131.29, 129.51, 123.15, 116.68, 113.94, 112.54, 107.20, 106.37, 49.49, 45.02.

### 4-(4-(4-phenylpiperazin-1-yl)pyrido[3,2-d]pyrimidin-6-yl)pyridin-2-amine, 4

Compound 4 was prepared via General Scheme 1 via the two step procedure reported for the preparation of compound 1. The two steps are reported below.

### 6-chloro-4-(4-phenylpiperazin-1-yl)pyrido[3,2-d]pyrimidine, 93

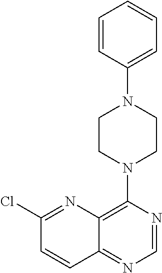 [SMILES: Clc1ccc2ncnc(N3CCN(c4ccccc4)CC3)c2n1]

In a 5 mL Biotage microwave vial, a stir bar, Sodium bicarbonate (33 mg, 15 μL, 1 eq, 400 μmol) and 1-phenylpiperazine (71 mg, 67 μL, 1.1 eq, 440 μmol), were added with EtOH (1.0 mL, 0.4 M) and stirred for 10 min. 4,6-dichloropyrido[3,2-d]pyrimidine (80 mg, 1 eq, 0.40 mmol) was added, the vial was sealed and heated at 25° C. for 12 hours. Upon completion, the reaction was diluted with water and filtered afford 6-chloro-4-(4-phenylpiperazin-1-yl)pyrido[3,2-d]pyrimidine, 93, (0.11 g, 0.34 mmol, 84%), as a white solid. LCMS [M+H]⁺ 326. 1H NMR (500 MHz, DMSO) δ 8.59 (d, J=2.8 Hz, 1H), 8.20 (dd, J=8.7, 2.8 Hz, 1H), 7.89 (dd, J=8.7, 2.8 Hz, 1H), 7.24 (t, J=7.8 Hz, 2H), 7.00 (d, J=8.0 Hz, 2H), 6.81 (t, J=7.3 Hz, 1H), 4.50 (s, 4H), 3.35 (t, J=4.3 Hz, 4H). ¹³C NMR (126 MHz, DMSO) δ 158.10, 155.45, 151.12, 146.92, 145.43, 140.47, 132.75, 129.46, 119.53, 115.89, 48.65, 40.50, 40.33, 40.16, 40.00, 39.83, 39.66, 39.50.

### 4-(4-(4-phenylpiperazin-1-yl)pyrido[3,2-d]pyrimidin-6-yl)pyridin-2-amine, 4

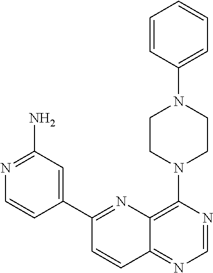 [SMILES: Nc1cc(-c2ccc3ncnc(N4CCN(c5ccccc5)CC4)c3n2)ccn1]

A 5 mL Biotage© microwave vial loaded with sodium bicarbonate (83 mg, 4.0 eq, 994 μmol), 1,1′-Bis(diphenylphosphino)ferrocenepalladium(II) dichloride (1 mg, 0.08 eq, 19 μmol), 6-chloro4-(4-phenylpiperazin-1-yl)pyrido[3,2-d]pyrimidine (81 mg, 1 eq, 249 μmol), and 4-(4,4,5,5-tetramethyl-1,3,2-dioxaborolan-2-yl)pyridin-2-amine (65 mg, 1.2 eq, 298 mol). The vial was capped, purged with argon, then injected with degassed 4:1 Dioxane:H₂O (2.5 mL, 0.1 M), and heated to 80° C. for 12 h. The reaction was cooled, diluted with DCM/MeOH, filtered through celite, concentrated, dry-loaded onto silica gel and purified on a 12 g silica gel column (DCM/MeOH, 0-20%), affording 4-(4-(4-phenylpiperazin-1-yl)pyrido[3,2-d]pyrimidin-6-yl)pyridin-2-amine, 4, (90 mg, 0.23 mmol, 94%) as a brown solid, LCMS [M+H]⁺ 384. 1H NMR (500 MHz, DMSO) δ 8.58 (d, J=2.1 Hz, 1H), 8.33 (dd, J=8.9, 2.2 Hz, 1H), 8.24 (dd, J=8.9, 2.2 Hz, 1H), 8.13-8.08 (m, 1H), 7.29-7.21 (m, 3H), 7.19 (s, 1H), 7.03 (d, J=8.0 Hz, 2H), 6.81 (t, J=7.3 Hz, 1H), 6.31 (s, 2H), 4.64 (s, 4H), 3.43 (t, J=4.9 Hz, 4H). 13C NMR (126 MHz, DMSO) δ 160.89, 159.11, 155.31, 151.16, 147.23, 146.40, 137.69, 133.33, 129.49, 125.15, 119.44, 115.81, 109.85, 105.65, 48.69, 40.48, 40.31, 40.14, 39.98, 39.81, 39.64, 39.48.

### 4-(4-(4-(pyridin-2-yl)piperazin-1-yl)pyrido[3,2-d]pyrimidin-6-yl)pyridin-2-amine, 5

Compound 5 was prepared via General Scheme 1 via the two step procedure reported for the preparation of compound 1. The two steps are reported below.

### 6-chloro-4-(4-(pyridin-2-yl)piperazin-1-yl)pyrido[3,2-d]pyrimidine, 94

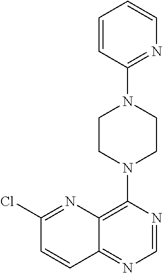 [SMILES: Clc1ccc2ncnc(N3CCN(c4ccccn4)CC3)c2n1]

In a 5 mL Biotage microwave vial, a stir bar, sodium bicarbonate (33 mg, 15 μL, 1 eq, 400 μmol) and 1-(pyridin-2-yl)piperazine (65 mg, 61 μL, 1.0 eq, 400 μmol), were added with EtOH (1.0 mL, 0.4 M) and stirred for 10 min. 4,6-dichloropyrido[3,2-d]pyrimidine (8 mg, 1 eq, 0.4 mmol) was added, the vial was sealed and stirred at 25° C. for 12 h. Upon completion, the reaction was diluted with water and filtered afford 6-chloro-4-(4-(pyridin-2-yl)piperazin-1-yl)pyrido[3,2-d]pyrimidine, 94, (0.11 g, 0.34 mmol, 84%) as a white solid. LCMS [M+H]⁺ 327. 1H NMR (500 MHz, DMSO) δ 8.60 (s, 1H), 8.20 (d, J=8.8 Hz, 1H), 8.15 (dd, J=5.0, 1.9 Hz, 1H), 7.90 (d, J=8.7 Hz, 1H), 7.57 (ddd, J=9.0, 7.0, 2.0 Hz, 1H), 6.89 (d, J=8.6 Hz, 1H), 6.68 (dd, J=7.1, 4.9 Hz, 1H), 4.46 (s, 4H), 3.75-3.69 (m, 4H). 13C NMR (126 MHz, DMSO) δ 159.16, 158.21, 155.46, 148.05, 146.91, 145.44, 140.42, 138.08, 132.78, 129.43, 113.64, 107.55, 44.84, 40.50, 40.33, 40.16, 40.00, 39.83, 39.66, 39.50.

### 4-(4-(4-(pyridin-2-yl)piperazin-1-yl)pyrido[3,2-d]pyrimidin-6-yl)pyridin-2-amine, 5

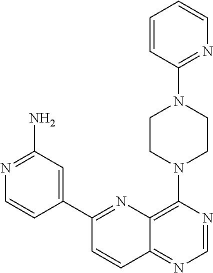 [SMILES: Nc1cc(-c2ccc3ncnc(N4CCN(c5ccccn5)CC4)c3n2)ccn1]

A 5 mL Biotage© microwave vial loaded with sodium bicarbonate (83 mg, 4 eq, 991 μmol), 1,1′-Bis(diphenylphosphino)ferrocenepalladium(II) dichloride (14 mg, 0.08 eq, 19 μmol), 6-chloro4-(4-(pyridin-2-yl)piperazin-1-yl)pyrido[3,2-d]pyrimidine (0.0810 g, 1 eq, 248 mol), and 4-(4,4,5,5-tetramethyl-1,3,2-dioxaborolan-2-yl)pyridin-2-amine (65 mg, 1.2 eq, 297 μmol). The vial was capped, purged with argon, then injected with degassed 4:1 Dioxane:H₂O (2.5 mL, 0.1 M), and heated to 80° C. for 12 h. The reaction was cooled, diluted with DCM/MeOH, filtered through celite, concentrated, dry-loaded onto silica gel and purified on a 12g silica gel column (DCM/MeOH, 0-20%), affording 4-(4-(4-(pyridin-2-yl)piperazin-1-yl)pyrido[3,2-d]pyrimidin-6-yl)pyridin-2-amine, 5, (90 mg, 0.23 mmol, 94%) as a brown solid. LCMS [M+H]⁺ 385. 1H NMR (500 MHz, DMSO) δ 8.58 (s, 1H), 8.33 (d, J=8.7 Hz, 1H), 8.23 (d, J=8.8 Hz, 1H), 8.16 (d, J=4.8 Hz, 1H), 8.11 (d, J=5.3 Hz, 1H), 7.59 (t, J=7.9 Hz, 1H), 7.25-7.18 (m, 2H), 6.90 (d, J=8.6 Hz, 1H), 6.68 (t, J=6.1 Hz, 1H), 6.33 (s, 2H), 4.60 (s, 4H), 3.79 (t, J=5.1 Hz, 4H). 13C NMR (126 MHz, DMSO) δ 160.85, 159.15, 155.30, 151.76, 148.78, 148.05, 147.22, 146.45, 138.09, 137.63, 133.37, 125.13, 113.54, 109.86, 107.43, 105.74, 44.88, 40.48, 40.31, 40.24, 40.15, 39.98, 39.81, 39.65, 39.48.

### 4-(4-(4-(pyridin-4-yl)piperazin-1-yl)quinazolin-6-yl)pyridin-2-amine, 6

Compound 6 was prepared via General Scheme 1 via the two step procedure reported for the preparation of compound 1. The two steps are reported below.

### 6-bromo-4-(4-(pyridin-4-yl)piperazin-1-yl)quinazoline, 95

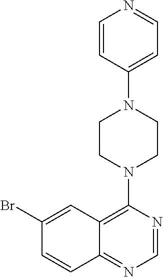 [SMILES: Brc1ccc2ncnc(N3CCN(c4ccncc4)CC3)c2c1]

In a 5 mL Biotage© microwave vial, a stir bar, sodium bicarbonate (42 mg, 1 eq, 500 μmol) and 1-(pyridin-4-yl)piperazine (97 mg, 1.2 eq, 600 μmol), and EtOH (2.0 mL, 0.4 M) were added stirred for 5 minutes. 6-bromo-4-chloroquinazoline (122 mg, 1 eq, 0.5 mmol) was added and the vial was sealed and heated at 50° C. for 12 hours. Upon completion, the reaction was diluted with water and extracted with DCM (10 mL×3) to afford 6-bromo-4-(4-(pyridin-4-yl)piperazin-1-yl)quinazoline, 82, (0.15 g, 0.41 mmol, 81%) as a white solid. LCMS [M+H]⁺ 370. This compound was used without further purification or characterization.

### 4-(4-(4-(pyridin-4-yl)piperazin-1-yl)quinazolin-6-yl)pyridin-2-amine, 6

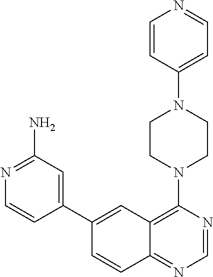 [SMILES: Nc1cc(-c2ccc3ncnc(N4CCN(c5ccncc5)CC4)c3c2)ccn1]

A 5 mL Biotage© microwave vial loaded with sodium bicarbonate (90 mg, 4.0 eq, 1.08 mmol), 1,1′-Bis(diphenylphosphino)ferrocenepalladium(II) dichloride (15 mg, 0.08 eq, 21 μmol), 6-bromo4-(4-(pyridin-4-yl)piperazin-1-yl)quinazoline (0.1 g, 1 eq, 270 μmol), and 4-(4,4,5,5-tetramethyl-1,3,2-dioxaborolan-2-yl)pyridin-2-amine (71 mg, 1.2 eq., 324 mol). The vial was capped, purged with argon, then injected with degassed 4:1 MeOH:H₂O (2.7 mL, 0.1 M), and heated to 80° C. for 12 h. The reaction was cooled, diluted with DCM/MeOH, filtered through celite, concentrated, dry-loaded onto silica gel and purified on a 12 g silica gel column (DCM/MeOH, 0-30%), affording 4-(4-(4-(pyridin-4-yl)piperazin-1-yl)quinazolin-6-yl)pyridin-2-amine, 6, (96 mg, 0.25 mmol, 93%) as a brown solid. LCMS [M+H]384. 1H NMR (500 MHz, DMSO) δ 8.67 (s, 1H), 8.24 (s, 2H), 8.10 (d, J=8.9 Hz, 1H), 8.03 (d, J=5.3 Hz, 1H), 7.91 (d, J=8.7 Hz, 1H), 6.93 (t, J=7.2 Hz, 3H), 6.86 (s, 1H), 6.09 (s, 2H), 4.07-4.01 (m, 4H), 3.71 (t, J=5.0 Hz, 5H). 13C NMR (126 MHz, DMSO) δ 163.73, 161.03, 155.07, 154.46, 151.94, 149.15, 147.94, 147.52, 135.56, 131.49, 129.22, 123.42, 116.22, 110.52, 105.72, 48.49, 45.25, 40.48, 40.31, 40.14, 39.98, 39.81, 39.64, 39.48.

### 6-(1-methyl-1H-pyrazol-4-yl)-4-(4-(pyridin-2-yl)piperazin-1-yl)quinazoline, 7

Compound 7 was prepared via General Scheme 1 via the two step procedure reported for the preparation of compound 1. The second step is reported below.

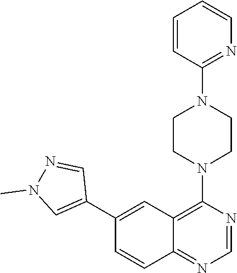 [SMILES: Cn1cc(-c2ccc3ncnc(N4CCN(c5ccccn5)CC4)c3c2)cn1]

A 5 mL Biotage© microwave vial loaded with sodium bicarbonate (68 mg, 4.0 eq, 0.81 mmol), 1,1′-Bis(diphenylphosphino)ferrocene-palladium(II) dichloride (12 mg, 0.08 eq, 16 μmol), 6-bromo-4-(4-(pyridin-2-yl)piperazin-1-yl)quinazoline (0.075 g, 1 eq, 0.20 mmol), and 1-methyl-4-(4,4,5,5-tetramethyl-1,3,2-dioxaborolan-2-yl)-1H-pyrazole (51 mg, 1.2 eq, 0.24 mmol). The vial was capped, purged with argon, then injected with degassed 4:1 Dioxane:H₂O (2.0 mL, 0.1 M), and heated to 160° C. for 25 min. The reaction was cooled, diluted with DCM/MeOH, filtered through celite, concentrated, dry-loaded onto silica gel and purified on a 12 g silica gel column (DCM/MeOH, 0-20%), affording 6-(1-methyl-1H-pyrazol-4-yl)-4-(4-(pyridin-2-yl)piperazin-1-yl)quinazoline, 7, (61 mg, 0.16 mmol, 81%) as a tan solid. LCMS [M+H]⁺ 372. 1H NMR (500 MHz, DMSO) δ 8.53 (s, 1H), 8.27 (s, 1H), 8.09 (dd, J=5.0, 2.0 Hz, 1H), 8.02 (d, J=2.0 Hz, 1H), 7.99 (dd, J=8.6, 1.9 Hz, 1H), 7.97 (s, 1H), 7.74 (d, J=8.6 Hz, 1H), 7.52 (ddd, J=8.9, 7.0, 2.0 Hz, 1H), 6.80 (d, J=8.6 Hz, 1H), 6.61 (dd, J=7.1, 4.9 Hz, 1H), 3.83 (d, J=5.6 Hz, 7H), 3.71 (dd, J=6.8, 3.7 Hz, 4H). 13C NMR (126 MHz, DMSO) δ 163.97, 159.27, 153.46, 148.09, 138.09, 136.96, 131.14, 130.53, 129.10, 128.98, 121.65, 120.03, 116.87, 113.61, 107.48, 49.24, 44.70, 40.37, 40.29, 40.21, 40.04, 39.87, 39.70, 39.54, 39.22.

### 6-(2-(difluoromethyl)pyridin-4-yl)-4-(4-(pyridin-2-yl)piperazin-1-yl)quinazoline, 8

Compound 8 was prepared via General Scheme 1 via the two step procedure reported for the preparation of compound 1. The second step is reported below.

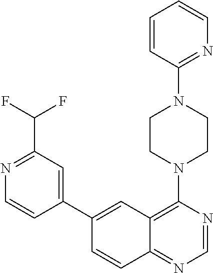 [SMILES: FC(F)c1cc(-c2ccc3ncnc(N4CCN(c5ccccn5)CC4)c3c2)ccn1]

The boronate 87 was employed in this procedure. A 5 mL Biotage© microwave vial loaded with sodium bicarbonate (90 mg, 4.0 eq, 1.08 mmol), 1,1′-Bis(diphenylphosphino)ferrocene-palladium(II) dichloride (15 mg, 0.08 eq, 21 mol), 6-bromo-4-(4-(pyridin-2-yl)piperazin-1-yl)quinazoline (100 mg, 1.0 eq, 270 μmol), and 2-(difluoromethyl)-4-(4,4,5,5-tetramethyl-1,3,2-dioxaborolan-2-yl)pyridine (75 mg, 1.1 eq, 297 μmol). The vial was capped, purged with argon, then injected with degassed 4:1 Dioxane:H₂O (2.7 mL, 0.1 M), and heated to 160° C. for 25 min. The reaction was cooled, diluted with DCM/MeOH, filtered through celite, concentrated, dry-loaded onto silica gel and purified on a 12 g silica gel column (DCM/MeOH, 0-20%), affording 6-(2-(difluoromethyl)pyridin-4-yl)-4-(4-(pyridin-2-yl)piperazin-1-yl)quinazoline, 8, (58 mg, 0.14 mmol, 51%) as a brown solid. LCMS [M+H]⁺ 419. 1H NMR (500 MHz, DMSO) δ 8.80 (d, J=5.2 Hz, 1H), 8.67 (s, 1H), 8.42 (d, J=2.1 Hz, 1H), 8.29 (dd, J=8.7, 2.0 Hz, 1H), 8.18-8.11 (m, 2H), 8.11-8.04 (m, 1H), 7.95 (d, J=8.7 Hz, 1H), 7.58 (ddd, J=8.9, 7.1, 2.0 Hz, 1H), 7.05 (s, 1H), 6.84 (d, J=8.6 Hz, 1H), 6.68 (dd, J=7.1, 4.9 Hz, 1H), 4.11-4.00 (m, 4H), 3.80-3.76 (m, 4H). 13C NMR (126 MHz, DMSO) δ 163.95, 159.13, 154.90, 152.50, 150.97, 148.33, 148.10, 138.10, 133.57, 131.78, 129.44, 124.80, 124.10, 118.76, 116.28, 114.26, 113.56, 107.39, 49.03, 44.58, 40.54, 40.38, 40.21, 40.04, 39.88, 39.71, 39.54, 0.57.

### 4-(4-(4-(pyrimidin-4-yl)piperazin-1-yl)quinazolin-6-yl)pyridin-2-amine, 9

Compound 9 was prepared via General Scheme 1 via the two step procedure reported for the preparation of compound 1. The two steps are reported below.

### 6-bromo-4-(4-(pyrimidin-4-yl)piperazin-1-yl)quinazoline, 96

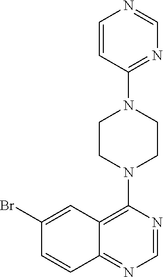 [SMILES: Brc1ccc2ncnc(N3CCN(c4ccncn4)CC3)c2c1]

In a 5 mL Biotage© microwave vial, a stir bar, sodium bicarbonate (42 mg, 1 eq, 500 μmol), 4-(piperazin-1-yl)pyrimidine (98 mg, 1.2 eq, 600 μmol), and EtOH (2.0 mL, 0.4 M) were added and stirred for 5 minutes. 6-bromo-4chloroquinazoline (122 mg, 1 eq, 0.5 mmol) was added and the vial was sealed and heated at 50° C. for 12 hours. Upon completion, the reaction was diluted with water and extracted with DCM (10 mL×3) to afford 6-bromo-4-(4-(pyrimidin-4-yl)piperazin-1-yl)quinazoline, 96, (0.15 g, 0.40 mmol, 81%) as a yellow solid. LCMS [M+H]⁺ 371. This compound was used without further purification or characterization.

### 4-(4-(4-(pyrimidin-4-yl)piperazin-1-yl)quinazolin-6-yl)pyridin-2-amine, 9

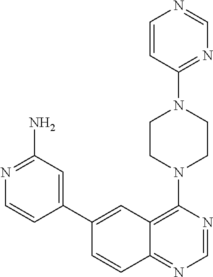 [SMILES: Nc1cc(-c2ccc3ncnc(N4CCN(c5ccncn5)CC4)c3c2)ccn1]

A 5 mL Biotage© microwave vial loaded with sodium bicarbonate (90 mg, 4.0 eq, 1.08 mmol), 1,1′-Bis(diphenylphosphino)ferrocene-palladium(II) dichloride (15 mg, 0.08 eq, 2 μmol), 6-bromo-4-(4-(pyrimidin-4-yl)piperazin-1-yl)quinazoline (0.1 g, 1 eq, 269 μmol), and 4-(4,4,5,5-tetramethyl-1,3,2-dioxaborolan-2-yl)pyridin-2-amine (71 mg, 1.2 eq. 324 μmol). The vial was capped, purged with argon, then injected with degassed 4:1 MeOH:H₂O (2.7 mL, 0.1 M), and heated to 80° C. for 12 h. The reaction was cooled, diluted with DCM/MeOH, filtered through celite, concentrated, dry-loaded onto silica gel and purified on a 12 g silica gel column (DCM/MeOH, 0-30%), affording 4-(4-(4-(pyrimidin-4-yl)piperazin-1-yl)quinazolin-6-yl)pyridin-2-amine, 9, (99 mg, 0.26 mmol, 96%) as a brown solid. LCMS [M+H]⁺ 385. 1H NMR (500 MHz, DMSO) δ 8.67 (s, 1H), 8.55 (s, 1H), 8.24 (d, J=8.5 Hz, 2H), 8.10 (d, J=8.7 Hz, 1H), 8.03 (d, J=5.4 Hz, 1H), 7.92 (d, J=8.6 Hz, 1H), 6.94 (d, J=5.4 Hz, 1H), 6.85 (d, J=15.2 Hz, 2H), 6.08 (s, 2H), 4.03-3.97 (m, 4H), 3.89 (t, J=5.2 Hz, 4H). 13C NMR (126 MHz, DMSO) δ 163.88, 161.32, 161.01, 158.36, 156.02, 154.46, 151.96, 149.21, 147.55, 135.64, 131.52, 129.24, 123.41, 110.56, 105.68, 79.65, 48.74, 43.18, 40.48, 40.31, 40.14, 39.98, 39.81, 39.64, 39.48.

### 4-(4-(4-(pyrimidin-2-yl)piperazin-1-yl)quinazolin-6-yl)pyridin-2-amine, 10

Compound 10 was prepared via General Scheme 1 via the two step procedure reported for the preparation of compound 1. The two steps are reported below.

### 6-bromo-4-(4-(pyrimidin-2-yl)piperazin-1-yl)quinazoline, 97

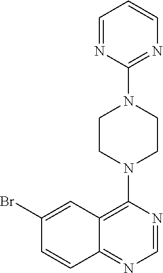 [SMILES: Brc1ccc2ncnc(N3CCN(c4ncccn4)CC3)c2c1]

In a 5 mL Biotage microwave vial, a stir bar, sodium bicarbonate (34 mg, 16 μL, 1 eq, 413 μmol), 2-(piperazin-1-yl)pyrimidine (8 mg, 1.2 eq, 496 mol), and EtOH (2.0 mL, 0.4 M) were added and stirred for 5 minutes. 6-bromo4-chloroquinazoline (100 mg, 1 eq, 413 mol) was added and the vial was sealed and heated at 50° C. for 12 hours. Upon completion, the reaction was diluted with water and filtered to afford 6-bromo-4-(4-(pyrimidin-2-yl)piperazin-1-yl)quinazoline, 97, (0.12 g, 0.32 mmol, 78%) as a white solid. LCMS [M+H]⁺ 371. This compound was used without further purification or characterization.

### 4-(4-(4-(pyrimidin-2-yl)piperazin-1-yl)quinazolin-6-yl)pyridin-2-amine, 10

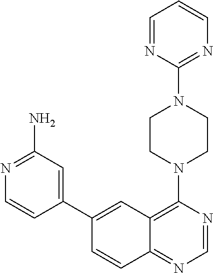 [SMILES: Nc1cc(-c2ccc3ncnc(N4CCN(c5ncccn5)CC4)c3c2)ccn1]

A 5 mL Biotage© microwave vial loaded with sodium bicarbonate (85 mg, 4.0 eq, 1.01 mmol), 1,1′-Bis(diphenylphosphino)ferrocenepalladium(II) dichloride (14 mg, 0.08 eq, 20 μmol), 6-bromo-4-(4-(pyrimidin-2-yl)piperazin-1-yl)quinazoline (0.0940 g, 1 eq, 253 μmol), and 4-(4,4,5,5-tetramethyl-1,3,2-dioxaborolan-2-yl)pyridin-2-amine (66 mg, 1.2 eq, 304 μmol). The vial was capped, purged with argon, then injected with degassed 4:1 MeOH:H₂O (2.5 mL, 0.1 M), and heated to 80° C. for 12 h. The reaction was cooled, diluted with DCM/MeOH, filtered through celite, concentrated, dry-loaded onto silica gel and purified on a 12 g silica gel column (DCM/MeOH, 0-30%), affording 4-(4-(4-(pyrimidin-2-yl)piperazin-1-yl)quinazolin-6-yl)pyridin-2-amine, 10, (0.091 g, 0.24 mmol, 93%) as a brown solid. LCMS [M+H]⁺ 385. 1H NMR (500 MHz, DMSO) δ 8.67 (s, 1H), 8.42 (d, J=4.7 Hz, 2H), 8.22 (s, 1H), 8.10 (dd, J=8.7, 1.9 Hz, 1H), 8.03 (d, J=5.3 Hz, 1H), 7.92 (d, J=8.7 Hz, 1H), 6.96 (d, J=5.4 Hz, 1H), 6.85 (s, 1H), 6.69 (t, J=4.8 Hz, 1H), 6.14 (s, 2H), 4.02-3.92 (m, 8H). 13C NMR (126 MHz, DMSO) δ 164.09, 161.59, 160.84, 158.48, 154.48, 151.97, 148.86, 147.74, 135.61, 131.53, 129.24, 123.44, 116.31, 110.89, 110.62, 105.86, 49.22, 43.49, 40.48, 40.31, 40.15, 39.98, 39.81, 39.65, 39.48.

### 4-(4-(4-(oxetan-3-yl)piperazin-1-yl)quinazolin-6-yl)pyridin-2-amine, 11

Compound 11 was prepared via General Scheme 1 via the two step procedure reported for the preparation of compound 1. The two steps are reported below.

### 6-bromo-4-(4-(oxetan-3-yl)piperazin-1-yl)quinazoline, 98

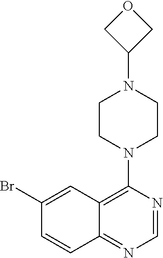 [SMILES: Brc1ccc2ncnc(N3CCN(C4COC4)CC3)c2c1]

In a 5 mL Biotage microwave vial, a stir bar, 1-(oxetan-3-yl)piperazine (70 mg, 64 μL, 1.2 eq, 493 μmol), sodium bicarbonate (34 mg, 1 eq. 411 μmol), and EtOH (2.0 mL, 0.2 M) were added stirred for 5 minutes. 6-bromo-4-chloroquinazoline (0.1 g, 1 eq, 411 mol) was added and the vial was sealed and heated at 50° C. for 12 hours. Upon completion, the reaction was diluted with water and extracted with DCM (10 mL×3) to afford 6-bromo-4-(4-(oxetan-3-yl)piperazin-1-yl)quinazoline, 98, (0.13 g, 0.37 mmol, 91%) as a tan solid. LCMS [M+H]⁺ 349. This compound was used without further purification or characterization.

### 4-(4-(4-(oxetan-3-yl)piperazin-1-yl)quinazolin-6-yl)pyridin-2-amine, 11

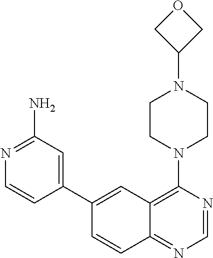 [SMILES: Nc1cc(-c2ccc3ncnc(N4CCN(C5COC5)CC4)c3c2)ccn1]

A 5 mL Biotage© microwave vial loaded with sodium bicarbonate (144 mg, 4.0 eq, 1.7 mmol), 1,1′-Bis(diphenylphosphino)ferrocenepalladium(II) dichloride (25 mg, 0.08 eq, 34 μmol), 6-bromo-4-(4-(oxetan-3-yl)piperazin-1-yl)quinazoline (0.15 g, 1 eq, 429 μmol), and 4-(4,4,5,5-tetramethyl-1,3,2-dioxaborolan-2-yl)pyridin-2-amine (104 mg, 1.1 eq, 472 μmol). The vial was capped, purged with argon, then injected with degassed 4:1 MeOH:H₂O (4.3 mL, 0.1 M), and heated to 80° C. for 12 h. The reaction was cooled, diluted with DCM/MeOH, filtered through celite, concentrated, dry-loaded onto silica gel and purified on a 12 g silica gel column (DCM/MeOH, 0-30%), affording 4-(4-(4-(oxetan-3-yl)piperazin-1-yl)quinazolin-6-yl)pyridin-2-amine, 11, (0.15 g, 0.41 mmol, 96%) as a brown solid. LCMS [M+H]⁺ 363. 1H NMR (500 MHz, DMSO) δ 8.65 (s, 1H), 8.11 (s, 1H), 8.07 (d, J=8.8 Hz, 1H), 8.03 (d, J=5.3 Hz, 1H), 7.90 (d, J=8.6 Hz, 1H), 6.90 (d, J=5.3 Hz, 1H), 6.80 (s, 1H), 6.12 (s, 2H), 4.58 (t, J=6.6 Hz, 2H), 4.49 (t, J=6.1 Hz, 2H), 3.84 (t, J=4.8 Hz, 4H), 3.50 (p, J=6.4 Hz, 1H), 2.48 (d, J=5.3 Hz, 4H). 13C NMR (126 MHz, DMSO) δ 164.21, 160.89, 154.48, 151.95, 149.03, 147.69, 135.73, 131.56, 129.31, 123.22, 116.28, 110.54, 105.76, 74.78, 58.81, 49.45, 49.42, 40.49, 40.32, 40.15, 39.99, 39.82, 39.65, 39.49.

### 6-(2-methylpyridin-4-yl)-4-(4-(pyridin-2-yl)piperazin-1-yl)quinazoline, 12

Compound 12 was prepared via General Scheme 1 via the two step procedure reported for the preparation of compound 1. The second step is reported below.

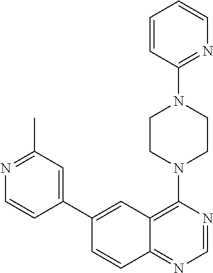 [SMILES: Cc1cc(-c2ccc3ncnc(N4CCN(c5ccccn5)CC4)c3c2)ccn1]

A 5 mL Biotage© microwave vial loaded with sodium bicarbonate (68 mg, 4.0 eq, 0.81 mmol), 1,1′-Bis(diphenylphosphino)ferrocene-palladium(II) dichloride (12 mg, 0.08 eq, 16 μmol), 6-bromo-4-(4-(pyridin-2-yl)piperazin-1-yl)quinazoline (0.075 g, 1 eq, 0.20 mmol), and 2-methyl-4-(4,4,5,5-tetramethyl-1,3,2-dioxaborolan-2-yl)pyridine (53 mg, 1.2 eq, 0.24 mmol). The vial was capped, purged with argon, then injected with degassed 4:1 Dioxane:H₂O (2.0 mL, 0.1 M), and heated to 160° C. for 25 min. The reaction was cooled, diluted with DCM/MeOH, filtered through celite, concentrated, dry-loaded onto silica gel and purified on a 12 g silica gel column (DCM/MeOH, 0-20%), affording 6-(2-methylpyridin-4-yl)-4-(4-(pyridin-2-yl)piperazin-1-yl)quinazoline, 12, (0.064 g, 0.17 mmol, 83%) as a brown solid. LCMS [M+H]⁺ 383. 1H NMR (500 MHz, DMSO) δ 8.67 (s, 1H), 8.56 (d, J=5.2 Hz, 1H), 8.35 (d, J=2.1 Hz, 1H), 8.23 (dd, J=8.7, 2.0 Hz, 1H), 8.16 (dd, J=4.8, 2.0 Hz, 1H), 7.93 (d, J=8.7 Hz, 1H), 7.73 (d, J=1.9 Hz, 1H), 7.66 (dd, J=5.3, 1.9 Hz, 1H), 7.59 (ddd, J=8.9, 7.1, 2.1 Hz, 1H), 6.86 (d, J=8.6 Hz, 1H), 6.68 (dd, J=7.1, 4.9 Hz, 1H), 4.04-3.99 (m, 4H), 3.81-3.75 (m, 4H), 2.58 (s, 3H). 13C NMR (126 MHz, DMSO) δ 163.98, 159.25, 159.14, 154.66, 152.19, 150.15, 148.09, 138.10, 131.76, 129.31, 124.08, 121.22, 119.19, 116.33, 113.56, 107.41, 49.08, 44.59, 40.54, 40.37, 40.21, 40.04, 39.87, 39.71, 39.54, 24.66.

### 6-(2-fluoropyridin-4-yl)-4-(4-(pyridin-2-yl)piperazin-1-yl)quinazoline, 13

Compound 13 was prepared via General Scheme 1 via the two step procedure reported for the preparation of compound 1. The second step is reported below.

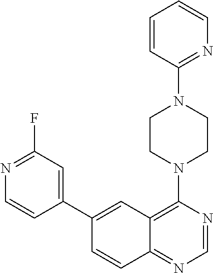 [SMILES: Fc1cc(-c2ccc3ncnc(N4CCN(c5ccccn5)CC4)c3c2)ccn1]

A 5 mL Biotage© microwave vial loaded with sodium bicarbonate (68 mg, 4.0 eq, 0.81 mmol), 1,1′ Bis(diphenylphosphino)ferrocene-palladium(II) dichloride (12 mg, 0.08 eq, 16 μmol), 6-bromo-4-(4-(pyridin-2-yl)piperazin-1-yl)quinazoline (0.075 g, 1 eq, 0.20 mmol), and 2-fluoro-4-(4,4,5,5-tetramethyl-1,3,2-dioxaborolan-2-yl)pyridine (54 mg, 1.2 eq, 0.24 mmol). The vial was capped, purged with argon, then injected with degassed 4:1 Dioxane:H₂O (2.0 mL, 0.1 M), and heated to 160° C. for 25 min. The reaction was cooled, diluted with DCM/MeOH, filtered through celite, concentrated, dry-loaded onto silica gel and purified on a 12 g silica gel column (DCM/MeOH, 0-20%), affording 6-(2-fluoropyridin-4-yl)-4-(4-(pyridin-2-yl)piperazin-1-yl)quinazoline, 13, (0.054 g, 0.14 mmol, 69%) as a creme solid. LCMS [M+H]⁺ 387. 1H NMR (500 MHz, DMSO) δ 8.72 (s, 1H), 8.47 (d, J=2.1 Hz, 1H), 8.42 (d, J=5.3 Hz, 1H), 8.33 (dd, J=8.7, 2.0 Hz, 1H), 8.21 (dd, J=4.9, 2.0 Hz, 1H), 7.99 (d, J=8.7 Hz, 1H), 7.91 (dd, J=5.2, 2.0 Hz, 1H), 7.76 (s, 1H), 7.64 (ddd, J=8.9, 7.1, 2.0 Hz, 1H), 6.90 (d, J=8.6 Hz, 1H), 6.73 (dd, J=7.1, 4.9 Hz, 1H), 4.11-4.06 (m, 4H), 3.86-3.80 (m, 4H). 13C NMR (126 MHz, DMSO) δ 163.89, 163.68, 159.13, 154.95, 152.60, 148.77, 148.65, 148.10, 138.09, 133.10, 131.76, 129.34, 124.90, 120.50, 116.19, 113.55, 107.68, 107.38, 48.99, 44.57, 40.54, 40.37, 40.20, 40.04, 39.87, 39.70, 39.54.

### 6-(1H-pyrazol-4-yl)-4-(4-(pyridin-2-yl)piperazin-1-yl)quinazoline, 14

Compound 14 was prepared via General Scheme 1 via the two step procedure reported for the preparation of compound 1. The second step is reported below.

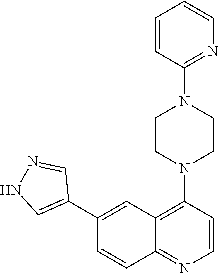 [SMILES: c1ccc(N2CCN(c3ccnc4ccc(-c5cn[nH]c5)cc34)CC2)nc1]

A 5 mL Biotage© microwave vial loaded with sodium bicarbonate (68 mg, 4.0 eq, 0.81 mmol), 1,1′-Bis(diphenylphosphino)ferrocene-palladium(II) dichloride (12 mg, 0.08 eq, 16 μmol), 6-bromo-4-(4-(pyridin-2-yl)piperazin-1-yl)quinazoline (0.075 g, 1 eq, 0.20 mmol), and 4-(4,4,5,5-tetramethyl-1,3,2-dioxaborolan-2-yl)-1H-pyrazole (47 mg, 1.2 eq, 0.24 mmol). The vial was capped, purged with argon, then injected with degassed 4:1 Dioxane:H₂O (2.0 mL, 0.1 M), and heated to 160° C. for 25 min. The reaction was cooled, diluted with DCM/MeOH, filtered through celite, concentrated, dry-loaded onto silica gel and purified on a 12 g silica gel column (DCM/MeOH, 0-20%), affording 6-(1H-pyrazol-4-yl)-4-(4-(pyridin-2-yl)piperazin-1-yl)quinazoline, 14, (52 mg, 0.15 mmol, 72%) as a tan solid. LCMS [M+H]⁺ 358. 1H NMR (500 MHz, DMSO) δ 13.12 (s, 1H), 8.66 (s, 1H), 8.45 (s, 1H), 8.24-8.14 (m, 4H), 7.87 (d, J=8.7 Hz, 1H), 7.64 (ddd, J=8.9, 7.1, 2.0 Hz, 1H), 6.92 (d, J=8.6 Hz, 1H), 6.74 (dd, J=7.1, 4.9 Hz, 1H), 3.96 (dd, J=6.8, 3.7 Hz, 4H), 3.83 (dd, J=6.7, 3.6 Hz, 4H). 13C NMR (126 MHz, DMSO) δ 163.95, 159.27, 153.41, 150.39, 148.09, 138.09, 131.41, 130.86, 129.01, 120.97, 120.17, 116.85, 113.59, 107.46, 49.23, 44.68, 40.54, 40.37, 40.20, 40.04, 39.87, 39.70, 39.54.

### 4-(4-(4-(2-fluorophenyl)piperazin-1-yl)quinazolin-6-yl)pyridin-2-amine, 15

Compound 15 was prepared via General Scheme 1 via the two step procedure reported for the preparation of compound 1. The second step is reported below.

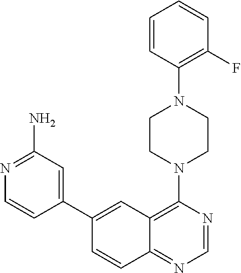 [SMILES: Nc1cc(-c2ccc3ncnc(N4CCN(c5ccccc5F)CC4)c3c2)ccn1]

A 5 mL Biotage© microwave vial loaded with sodium bicarbonate (9 mg, 4.0 eq, 1.08 mmol), 1,1′-Bis(diphenylphosphino)ferrocene-palladium(II) dichloride (15 mg, 0.08 eq, 2 μmol), 6-bromo-4-(4-(2-fluorophenyl)piperazin-1-yl)quinazoline (0.105 g, 1 eq, 271 mol), and 4-(4,4,5,5-tetramethyl-1,3,2-dioxaborolan-2-yl)pyridin-2-amine (6 mg, 1.1 eq, 298 mol). The vial was capped, purged with argon, then injected with degassed 4:1 MeOH:H₂O (2.8 mL, 0.1 M), and heated to 80° C. for 12 h. The reaction was cooled, diluted with DCM/MeOH, filtered through celite, concentrated, dry-loaded onto silica gel and purified on a 12 g silica gel column (DCM/MeOH, 0-30%), affording 4-(4-(4-(2-fluorophenyl)piperazin-1-yl)quinazolin-6-yl)pyridin-2-amine, 15, (77 mg, 0.19 mmol, 71%) as a creme solid. LCMS [M+H]⁺ 401. 1H NMR (500 MHz, DMSO) δ 8.61 (s, 1H), 8.12 (d, J=2.0 Hz, 1H), 8.02 (dd, J=8.7, 2.0 Hz, 1H), 7.96 (d, J=5.3 Hz, 1H), 7.85 (d, J=8.7 Hz, 1H), 7.14-7.09 (m, 1H), 7.05 (td, J=7.4, 4.8 Hz, 2H), 6.98-6.90 (m, 1H), 6.86 (dd, J=5.3, 1.7 Hz, 1H), 6.76 (s, 1H), 5.98 (s, 2H), 3.92 (t, J=4.8 Hz, 4H), 3.18 (t, J=4.7 Hz, 4H). 13C NMR (126 MHz, DMSO) δ 164.26, 161.01, 154.49, 151.99, 149.23, 147.56, 135.87, 131.60, 129.35, 125.39, 125.36, 123.19, 119.96, 116.59, 116.43, 116.36, 110.60, 105.71, 50.51, 49.75, 40.55, 40.38, 40.21, 40.05, 39.88, 39.71, 39.54.

### 4-(4-(4-(5-(trifluoromethyl)pyridin-2-yl)piperazin-1-yl)pyrido[3,2-d]pyrimidin-6-yl)pyridin-2-amine, 16

Compound 16 was prepared via General Scheme 1 via the two step procedure reported for the preparation of compound 1. The two steps are reported below.

### 6-chloro-4-(4-(5-(trifluoromethyl)pyridin-2-yl)piperazin-1-yl)pyrido[3,2-d]pyrimidine, 99

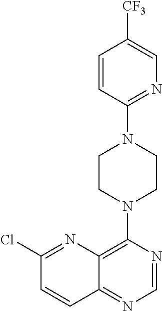 [SMILES: FC(F)(F)c1ccc(N2CCN(c3ncnc4ccc(Cl)nc34)CC2)nc1]

In a 5 mL Biotage microwave vial, sodium bicarbonate (63 mg, 1 eq, 750 mol) and 1-(5-(trifluoromethyl)pyridin-2-yl)piperazine (173 mg, 1.0 eq, 750 μmol), were added with EtOH (1.8 mL, 0.4 M) and stirred for 10 min. 4,6-dichloropyrido[3,2-d]pyrimidine (150 mg, 1 eq, 750 μmol mol) was added, the vial was sealed, and stirred at 25° C. for 12 hours. Upon completion, the reaction was diluted with water and filtered to afford 6-chloro-4-(4-(5-(trifluoromethyl)pyridin-2-yl)piperazin-1-yl)pyrido[3,2-d]pyrimidine, 99, (0.187 g, 0.474 μmol, 63%) as a white solid. LCMS [M+H]⁺ 395. 1H NMR (500 MHz, DMSO) δ 8.60 (s, 1H), 8.48-8.42 (m, 1H), 8.20 (d, J=8.8 Hz, 1H), 7.89 (d, J=8.8 Hz, 1H), 7.85 (dd, J=9.1, 2.6 Hz, 1H), 7.00 (d, J=9.1 Hz, 1H), 4.46 (s, 4H), 3.97-3.76 (m, 4H).

### 4-(4-(4-(5-(trifluoromethyl)pyridin-2-yl)piperazin-1-yl)pyrido[3,2-d]pyrimidin-6-yl)pyridin-2-amine, 16

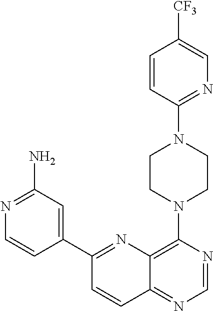 [SMILES: Nc1cc(-c2ccc3ncnc(N4CCN(c5ccc(C(F)(F)F)cn5)CC4)c3n2)ccn1]

In a 5 mL microwave vial, 6-chloro-4-(4-(5-(trifluoromethyl)pyridin-2-yl)piperazin-1-yl)pyrido[3,2-d]pyrimidine (54 mg, 1 eq, 0.14 mmol), 4-(4,4,5,5-tetramethyl-1,3,2-dioxaborolan-2-yl)pyridin-2-amine (36 mg, 1.2 eq, 0.16 mmol), tripotassium phosphate (87 mg, 3 eq, 0.41 mmol), and [1,1′-Bis(diphenylphosphino)ferrocene]dichloropalladium(II), complex with dichloromethane (9.0 mg, 0.08 eq, 11.0 μmol) were added. The mixture was capped, purged with argon, then injected with degassed 1,4-dioxane (0.8 mL) and water (0.2 mL), and was heated at 80° C. for 12 h. After this time, the mixture was cooled to room temperature, diluted with ethyl acetate (30 mL), and washed with water (10 mL×3). The aqueous layer was washed with ethyl acetate (20 mL×2). The combined organic layer was washed with brine (20 mL xx 2), dried over sodium sulfate, and evaporated in vacuo to obtain the crude product. The crude product was dry-loaded onto silica gel and purified on a 12 g silica gel column (0-20% MeOH/DCM) to afford 4-(4-(4-(5-(trifluoromethyl)pyridin-2-yl)piperazin-1-yl)pyrido[3,2-d]pyrimidin-6-yl)pyridin-2-amine, 16, (49 mg, 110 μmol, 81%) as a tan solid. LCMS [M+H]⁺ 452.95. ¹H NMR (500 MHz, CDCl₃) δ 8.66 (s, 1H), 8.44 (d, J=2.4 Hz, 1H), 8.23 (d, J=8.7 Hz, 1H), 8.20 (d, J=5.3 Hz, 1H), 8.09 (d, J=8.7 Hz, 1H), 7.69 (dd, J=9.0, 2.5 Hz, 1H), 7.28 (d, J=5.4 Hz, 1H), 7.17 (s, 1H), 6.70 (d, J=8.9 Hz, 1H), 5.06 (s, 2H), 4.69 (s, 4H), 3.94 (t, J=5.3 Hz, 4H). ¹³C NMR (126 MHz, CDCl₃) δ 160.21, 159.61, 158.87, 155.58, 151.47, 148.21, 147.48, 145.92, 137.66, 134.86, 133.80, 124.53, 123.58, 115.74, 111.81, 106.62, 105.64, 44.64.

### 4-(4-(pyridin-2-yl)piperazin-1-yl)-6-(1H-pyrrolo[2,3-b]pyridin-3-yl)quinazoline, 17

Compound 17 was prepared via General Scheme 1 via the two step procedure reported for the preparation of compound 1. The second step is reported below.

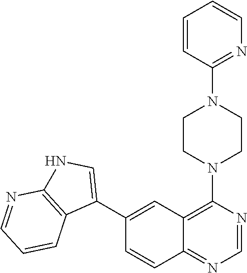 [SMILES: c1ccc(N2CCN(c3ncnc4ccc(-c5c[nH]c6ncccc56)cc34)CC2)nc1]

A 5 mL Biotage© microwave vial loaded with sodium bicarbonate (55 mg, 4.0 eq, 0.65 mmol), tert-butyl 3-(4,4,5,5-tetramethyl1,3,2-dioxaborolan-2-yl)-1H-pyrrolo[2,3-b]pyridine-1-carboxylate (62 mg, 1.1 eq, 0.18 mmol), [1,1′-Bis(diphenylphosphino) ferrocene]dichloropalladium (II) Complex With Dichloromethane (11 mg, 0.08 eq, 13 μmol), and 6-bromo-4-(4-(pyridin-2-yl)piperazin-1-yl)quinazoline (61 mg, 1.0 eq, 0.16 mmol). The vial was capped, purged with argon, then injected with degassed 1,4-Dioxane (1.5 mL, 0.14 M), and heated to 80° C. for 12 h. The reaction was cooled, concentrated, The reaction was cooled, diluted with DCM/MeOH, filtered through celite, concentrated, dry-loaded onto silica gel and purified on a 12 g silica gel column (DCM/MeOH, 0-20%), affording tert-butyl 3-(4-(4-(pyridin-2-yl)piperazin-1-yl)quinazolin-6-yl)-1H-pyrrolo[2,3-b]pyridine-1-carboxylate, LCMS [M+H]⁺ 508, which was then subjected to 1M HCl in EtOAc in which 4-(4-(pyridin-2-yl)piperazin-1-yl)-6-(1H-pyrrolo[2,3-b]pyridin-3-yl)quinazoline, 17, (23 mg, 56 μmol, 34%) precipitated out as tan solid LCMS [M+H]⁺ 408. 1H NMR (500 MHz, DMSO) δ 12.09 (s, 1H), 8.64 (s, 1H), 8.36 (d, J=8.0 Hz, 1H), 8.32 (d, J=4.5 Hz, 1H), 8.24 (d, J=6.8 Hz, 2H), 8.16 (d, J=4.8 Hz, 1H), 8.11 (s, 1H), 7.89 (d, J=9.0 Hz, 1H), 7.58 (t, J=7.6 Hz, 1H), 7.24 (dd, J=8.0, 4.6 Hz, 1H), 6.88 (d, J=8.6 Hz, 1H), 6.68 (dd, J=7.1, 4.9 Hz, 1H), 3.97-3.92 (m, 4H), 3.79 (t, J=4.9 Hz, 4H). 13C NMR (126 MHz, DMSO) δ 163.99, 159.27, 153.48, 150.28, 149.71, 148.10, 138.10, 133.00, 132.37, 129.13, 125.56, 121.13, 117.63, 116.94, 113.63, 107.49, 49.32, 44.77, 40.54, 40.38, 40.30, 40.21, 40.14, 40.04, 39.87, 39.71, 39.54, 0.57.

### 6-(1H-pyrazol-3-yl)-4-(4-(pyridin-2-yl)piperazin-1-yl)pyrido[3,2-d]pyrimidine, 18

Compound 18 was prepared via General Scheme 1 via the two step procedure reported for the preparation of compound 1. The second step is reported below.

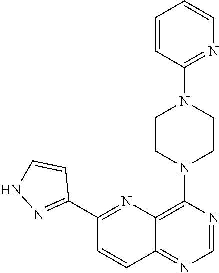 [SMILES: c1ccc(N2CCN(c3ncnc4ccc(-c5cc[nH]n5)nc34)CC2)nc1]

A 5 mL Biotage© microwave vial loaded with tripotassium phosphate (0.14 g, 3 eq, 0.68 mmol), [1,1′-Bis(diphenylphosphino)ferrocene]dichloropalladium(II), complex with dichloromethane (15 mg, 0.08 eq, 18 μmol), 6-chloro-4-(4-(pyridin-2-yl)piperazin-1-yl)pyrido[3,2-d]pyrimidine (74 mg, 1 eq, 0.23 mmol), and 4-(4,4,5,5-tetramethyl-1,3,2-dioxaborolan-2-yl)-1H-pyrazole (53 mg, 1.2 eq, 0.27 mmol). The vial was capped, purged with argon, then injected with degassed 4:1 Dioxane:H2O (2 mL, 0.1 M) and heated to 80° C. for 12 h. The reaction was cooled, diluted with DCM:MeOH, filtered through celite, concentrated, dry-loaded onto silica gel and purified on 12 g column (DCM/MeOH 0-20%), affording 6-(1H-pyrazol-4-yl)-4-(4-(pyridin-2-yl)piperazin-1-yl)pyrido[3,2-d]pyrimidine, 18, (17 mg, 48.0 μmol, 21%) as a pale yellow solid. LCMS [M+H]⁺ 356. 1H NMR (500 MHz, DMSO) δ 13.23 (s, 1H), 8.50 (d, J=5.7 Hz, 2H), 8.21-8.12 (m, 3H), 8.09 (d, J=8.8 Hz, 1H), 7.58 (ddd, J=8.8, 7.1, 2.0 Hz, 1H), 6.90 (d, J=8.6 Hz, 1H), 6.68 (dd, J=7.0, 4.9 Hz, 1H), 4.55 (s, 4H), 3.87-3.62 (m, 4H). 13C NMR (126 MHz, DMSO) δ 158.82, 158.38, 153.59, 149.17, 147.58, 145.46, 137.76, 137.58, 136.60, 132.73, 128.16, 124.77, 121.88, 113.08, 107.04, 44.55.

### 4-(4-(4-(2-fluorophenyl)piperazin-1-yl)pyrido[3,2-d]pyrimidin-6-yl)pyridin-2-amine, 19

Compound 19 was prepared via General Scheme 1 via the two step procedure reported for the preparation of compound 1. The two steps are reported below.

### 6-chloro-4-(4-(2-fluorophenyl)piperazin-1-yl)pyrido[3,2-d]pyrimidine, 100

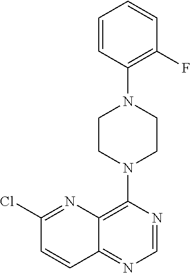 [SMILES: Fc1ccccc1N1CCN(c2ncnc3ccc(Cl)nc23)CC1]

In a 5 mL Biotage microwave vial, sodium bicarbonate (71 mg, 1 eq, 850 mol) and 1-(2-fluorophenyl)piperazine (153 mg, 134 μL, 1.0 eq, 850 μmol), were added with EtOH (2.0 mL, 0.4 M) and stirred for 10 min. 4,6-dichloropyrido[3,2-d]pyrimidine (170 mg, 1 eq, 850 μmol mol) was added, the vial was sealed and stirred at 25° C. for 12 h. Upon completion, the reaction was diluted with water and filtered to afford 6-chloro-4-(4-(2-fluorophenyl)piperazin-1-yl)pyrido[3,2-d]pyrimidine, 100, (0.193 g, 0.561 μmol, 66%) as a white solid. LCMS [M+H]⁺ 344. 1H NMR (500 MHz, DMSO) δ 8.59 (s, 1H), 8.20 (d, J=8.8 Hz, 1H), 7.88 (d, J=8.8 Hz, 1H), 7.21-7.13 (m, 1H), 7.09 (qd, J=7.4, 1.7 Hz, 2H), 7.00 (tdd, J=7.6, 4.7, 2.2 Hz, 1H), 4.51 (s, 4H), 3.20 (t, J=5.0 Hz, 4H).

### 4-(4-(4-(2-fluorophenyl)piperazin-1-yl)pyrido[3,2-d]pyrimidin-6-yl)pyridin-2-amine, 19

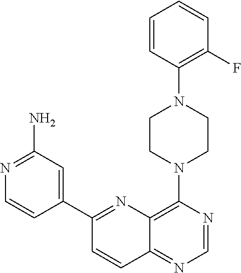 [SMILES: Nc1cc(-c2ccc3ncnc(N4CCN(c5ccccc5F)CC4)c3n2)ccn1]

In a 5 mL microwave vial, 6-chloro-4-(4-(2-fluorophenyl)piperazin-1-yl)pyrido[3,2-d]pyrimidine (40 mg, 1 eq, 0.12 mmol), 4-(4,4,5,5-tetramethyl-1,3,2-dioxaborolan-2-yl)pyridin-2-amine (31 mg, 1.2 eq, 0.14 mmol), tripotassium phosphate (99 mg, 4 eq, 0.47 mmol), and [1,1′-Bis(diphenylphosphino)ferrocene]dichloropalladium(II), complex with dichloromethane (7 mg, 0.08 eq, 9 μmol) were added. The mixture was capped, purged with argon, then injected with degassed 4:1 Dioxane:H₂O (0.7 mL, 0.2 M) and was heated at 80° C. for 12 h. After this time, the mixture was cooled to room temperature, diluted with ethyl acetate (30 mL), and washed with water (10 mL×3). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (0-20% MeOH/DCM) to afford 4-(4-(4-(2-fluorophenyl)piperazin-1-yl)pyrido[3,2-d]pyrimidin-6-yl)pyridin-2-amine, 19, (36 mg, 90 μmol, 78%) as a pale yellow solid. LCMS [M+H]⁺ 402. 1H NMR (500 MHz, CDCl3) δ 8.65 (s, 1H), 8.22 (d, J=8.7 Hz, 1H), 8.18 (d, J=5.6 Hz, 1H), 8.08 (d, J=8.9 Hz, 1H), 7.31-7.27 (d, 1H), 7.19 (s, 1H), 7.11-7.04 (m, 2H), 7.02-6.95 (m, 2H), 5.13 (s, 2H), 4.74 (s, 4H), 3.35-3.28 (t, 4H).

### 4-(4-(2-fluorophenyl)piperazin-1-yl)-6-(1-methyl-1H-pyrazol-3-yl)pyrido[3,2-d]pyrimidine, 20

Compound 20 was prepared via General Scheme 1 via the two step procedure reported for the preparation of compound 1. The second step is reported below.

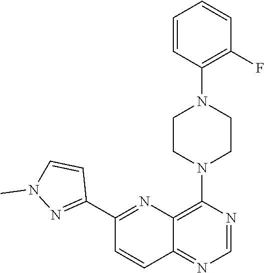 [SMILES: Cn1ccc(-c2ccc3ncnc(N4CCN(c5ccccc5F)CC4)c3n2)n1]

A 5 mL Biotage© microwave vial loaded with tripotassium phosphate (0.12 g, 4 eq, 0.58 mmol), [1,1′-Bis(diphenylphosphino)ferrocene]dichloropalladium(II), complex with dichloromethane (9 mg, 0.08 eq, 12 μmol), 6-chloro-4-(4-(2-fluorophenyl)piperazin-1-yl)pyrido[3,2-d]pyrimidine (50 mg, 1 eq, 0.15 mmol), and 1-methyl-4-(4,4,5,5-tetramethyl-1,3,2-dioxaborolan-2-yl)-1H-pyrazole (36 mg, 1.2 eq, 0.17 mmol). The vial was capped, purged with argon, then injected with degassed 4:1 Dioxane:H₂O (0.7 mL, 0.2 M) and heated to 80° C. for 12 h. The reaction was cooled, diluted with DCM:MeOH, filtered through celite, concentrated, dry-loaded onto silica gel and purified on 12 g column (DCM/MeOH 0-20%), affording 4-(4-(2-fluorophenyl)piperazin-1-yl)-6-(1-methyl-1H-pyrazol-3-yl)pyrido[3,2-d]pyrimidine, 20, (26 mg, 67 μmol, 46%) as a tan solid. LCMS [M+H]⁺ 390. 1H NMR (500 MHz, DMSO) δ 8.50 (s, 1H), 8.41 (s, 1H), 8.13 (s, 1H), 8.09 (s, 2H), 7.21-7.08 (m, 3H), 7.01 (dtd, J=8.1, 4.8, 3.1 Hz, 1H), 3.93 (d, J=4.0 Hz, 4H), 3.26 (t, J=5.0 Hz, 4H), 1.07 (s, 3H). 13C NMR (126 MHz, DMSO) δ 158.32, 156.00, 154.05, 153.55, 148.83, 145.49, 139.66, 139.60, 137.69, 136.75, 132.71, 130.29, 124.87, 124.85, 124.56, 122.66, 122.59, 122.36, 119.54, 116.06, 115.90, 73.49, 50.40, 24.94.

### 6-(1-methyl-1H-pyrazol-3-yl)-4-(4-(5-(trifluoromethyl)pyridin-2-yl)piperazin-1-yl)pyrido[3,2-d]pyrimidine, 21

Compound 21 was prepared via General Scheme 1 via the two step procedure reported for the preparation of compound 1. The second step is reported below.

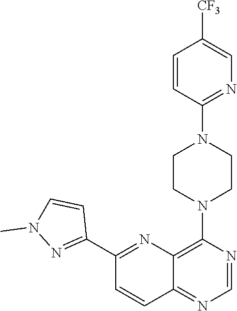 [SMILES: Cn1ccc(-c2ccc3ncnc(N4CCN(c5ccc(C(F)(F)F)cn5)CC4)c3n2)n1]

A 5 mL Biotage© microwave vial loaded with tripotassium phosphate (0.11 g, 4 eq, 0.51 mmol), [1,1′-Bis(diphenylphosphino)ferrocene]dichloropalladium(II), complex with dichloromethane (8 mg, 0.08 eq, 10 μmol), 6-chloro-4-(4-(5-(trifluoromethyl)pyridin-2-yl)piperazin-1-yl)pyrido[3,2-d]pyrimidine (50 mg, 1 eq, 0.13 mmol), and 1-methyl-4-(4,4,5,5-tetramethyl-1,3,2-dioxaborolan-2-yl)-1H-pyrazole (32 mg, 1.2 eq, 0.15 mmol). The vial was capped, purged with argon, then injected with degassed 4:1 Dioxane:H₂O (0.7 mL, 0.2 M) and heated to 80° C. for 12 h. The reaction was cooled, diluted with DCM:MeOH, filtered through celite, concentrated, dry-loaded onto silica gel and purified on 12 g column (DCM/MeOH 0-20%), affording 6-(1-methyl-1H-pyrazol-3-yl)-4-(4-(5-(trifluoromethyl)pyridin-2-yl)piperazin-1-yl)pyrido[3,2-d]pyrimidine, 21, (21 mg, 48 μmol, 39%) as a white solid. LCMS [M+H]⁺ 441. 1H NMR (500 MHz, DMSO) δ 8.50 (s, 1H), 8.47-8.45 (m, 1H), 8.44 (s, 1H), 8.18-8.13 (m, 1H), 8.10 (s, 2H), 7.86 (dd, J=9.1, 2.6 Hz, 1H), 7.01 (d, J=9.1 Hz, 1H), 4.56 (s, 4H), 3.94 (s, 3H), 3.92 (t, J=5.3 Hz, 4H).

### 6-(1H-pyrazol-3-yl)-4-(4-(5-(trifluoromethyl)pyridin-2-yl)piperazin-1-yl)pyrido[3,2-d]pyrimidine, 22

Compound 22 was prepared via General Scheme 1 via the two step procedure reported for the preparation of compound 1. The second step is reported below.

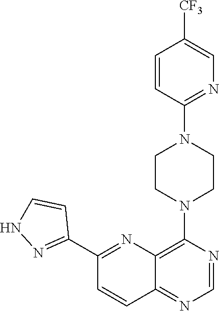 [SMILES: FC(F)(F)c1ccc(N2CCN(c3ncnc4ccc(-c5cc[nH]n5)nc34)CC2)nc1]

A 5 mL Biotage© microwave vial loaded with tripotassium phosphate (0.15 g, 4 eq, 0.71 mmol), [1,1′-Bis(diphenylphosphino)ferrocene]dichloropalladium(II), complex with dichloromethane (12 mg, 0.08 eq, 14 μmol), 6-chloro-4-(4-(5-(trifluoromethyl)pyridin-2-yl)piperazin-1-yl)pyrido[3,2-d](70 mg, 1 eq, 0.18 mmol), and tert-butyl 4-(4,4,5,5-tetramethyl-1,3,2-dioxaborolan-2-yl)-1H-pyrazole-1-carboxylate (63 mg, 1.2 eq, 0.21 mmol). The vial was capped, purged with argon, then injected with degassed 4:1 Dioxane:H₂O (1.0 mL, 0.2 M) and heated to 80° C. for 12 h. The reaction was cooled, diluted with DCM:MeOH, filtered through celite, and concentrated. A mixture of DCM:TFA (1:2, 1 mL) was added to the crude mixture and stirred at room temperature overnight and basified with 1N NaOH. The mixture was extracted with DCM, washed with 10% NaOH, water, brine, and the organic layer was dried over sodium sulfate, concentrated, and dry-loaded onto silica gel and purified on a 12 g column (DCM/MeOH 0-20%), affording 6-(1H-pyrazol-3-yl)-4-(4-(5-(trifluoromethyl)pyridin-2-yl)piperazin-1-yl)pyrido[3,2-d]pyrimidine, 22, (14 mg, 34 μmol, 19%) as a tan solid. LCMS [M+H]⁺ 427. 1H NMR (500 MHz, DMSO) δ 13.23 (s, 1H), 8.52 (s, 1H), 8.51 (s, 1H), 8.48-8.44 (m, 1H), 8.20 (d, J=1.9 Hz, 1H), 8.15 (d, J=8.8 Hz, 1H), 8.10 (d, J=8.8 Hz, 1H), 7.85 (dd, J=9.1, 2.6 Hz, 1H), 7.02 (d, J=9.1 Hz, 1H), 4.56 (s, 4H), 4.00-3.82 (m, 4H).

### 4-(4-(2-fluorophenyl)piperazin-1-yl)-6-(1H-pyrazol-3-yl)pyrido[3,2-d]pyrimidine, 23

Compound 23 was prepared via General Scheme 1 via the two step procedure reported for the preparation of compound 1. The second step is reported below.

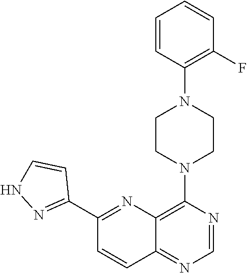 [SMILES: Fc1ccccc1N1CCN(c2ncnc3ccc(-c4cc[nH]n4)nc23)CC1]

A 5 mL Biotage© microwave vial loaded with tripotassium phosphate (0.17 g, 4 eq, 0.81 mmol), [11,1′-Bis(diphenylphosphino)ferrocene]dichloropalladium(II), complex with dichloromethane (13 mg, 0.08 eq, 16 μmol), 6-chloro-4-(4-(2-fluorophenyl)piperazin-1-yl)pyrido[3,2-d]pyrimidine (70 mg, 1 eq, 0.20 mmol), and tert-butyl 4-(4,4,5,5-tetramethyl-1,3,2-dioxaborolan-2-yl)-1H-pyrazole-1-carboxylate (72 mg, 1.2 eq, 0.24 mmol). The vial was capped, purged with argon, then injected with degassed 4:1 Dioxane:H2O (1.0 mL, 0.2 M) and heated to 80° C. for 12 h. The reaction was cooled, diluted with DCM:MeOH, filtered through celite, and concentrated. A mixture of DCM:TFA (1:2, 1.2 mL) was added to the crude mixture and stirred at room temperature overnight and basified with 1N NaOH. The mixture was extracted with DCM, washed with 10% NaOH, water, brine, and the organic layer was dried over sodium sulfate, concentrated, and dry loaded onto silica gel and purified on a 12 g column (DCM/MeOH 0-20%), affording 4-(4-(2-fluorophenyl)piperazin-1-yl)-6-(1H-pyrazol-3-yl)pyrido[3,2-d], 23, (64 mg, 173 μmol, 85%) as a yellow/orange solid. LCMS [M+H]⁺ 376. 1H NMR (500 MHz, DMSO) δ 8.76 (s, 1H), 8.40 (s, 2H), 8.29 (d, J=8.7 Hz, 1H), 8.17 (d, J=8.8 Hz, 1H), 7.23-7.14 (m, 2H), 7.13 (d, J=2.6 Hz, 1H), 7.02 (dtd, J=8.0, 5.0, 3.1 Hz, 1H), 4.80 (s, 4H), 3.35 (s, 4H). 13C NMR (126 MHz, DMSO) δ 158.73, 158.46, 156.41, 154.47, 151.18, 150.35, 139.64, 139.58, 138.67, 131.75, 131.52, 126.75, 125.40, 125.37, 123.32, 123.26, 121.65, 120.07, 120.05, 116.63, 116.47, 50.76.

### 4-(4-(4-(4-fluorophenyl)piperazin-1-yl)pyrido[3,2-d]pyrimidin-6-yl)pyridin-2-amine, 24

Compound 24 was prepared via General Scheme 1 via the two step procedure reported for the preparation of compound 1. The two steps are reported below.

### 6-chloro-4-(4-(4-fluorophenyl)piperazin-1-yl)pyrido[3,2-d]pyrimidine, 101

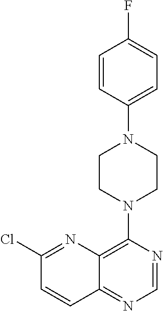 [SMILES: Fc1ccc(N2CCN(c3ncnc4ccc(Cl)nc34)CC2)cc1]

In a 5 mL Biotage microwave vial, sodium bicarbonate (55 mg, 1 eq. 760 mol) and 1-(4-fluorophenyl)piperazine (117 mg, 1.0 eq, 650 μmol), were added with EtOH (1.6 mL, 0.4 M) and stirred for 10 min. 4,6-dichloropyrido[3,2-d]pyrimidine (130 mg, 1 eq, 650 μmol) was added, the vial was sealed, and stirred at 25° C. for 12 h. Upon completion, the reaction was diluted with water and filtered to afford 6-chloro-4-(4-(4-fluorophenyl)piperazin-1-yl)pyrido[3,2-d]pyrimidine, 101, (0.140 g, 0.407 μmol, 63%). LCMS [M+H]⁺ 344. 1H NMR (500 MHz, DMSO) δ 8.59 (s, 1H), 8.19 (d, J=8.8 Hz, 1H), 7.89 (d, J=8.8 Hz, 1H), 7.08 (t, J=8.8 Hz, 2H), 7.02 (dd, J=9.2, 4.7 Hz, 2H), 4.64-4.38 (s, 4H), 3.29 (t, J=5.2 Hz, 4H).

### 4-(4-(4-(4-fluorophenyl)piperazin-1-yl)pyrido[3,2-d]pyrimidin-6-yl)pyridin-2-amine, 24

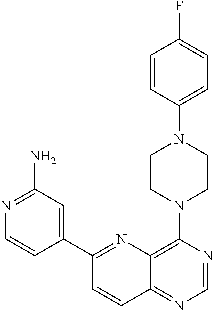 [SMILES: Nc1cc(-c2ccc3ncnc(N4CCN(c5ccc(F)cc5)CC4)c3n2)ccn1]

In a 5 mL microwave vial, 6-chloro-4-(4-(4-fluorophenyl)piperazin-1-yl)pyrido[3,2-d]pyrimidine (50 mg, 1 eq, 0.15 mmol), 4-(4,4,5,5-tetramethyl-1,3,2-dioxaborolan-2-yl)pyridin-2-amine (38 mg, 1.2 eq, 0.17 mmol), tripotassium phosphate (120 mg, 4 eq, 0.58 mmol), and [1,1′-Bis(diphenylphosphino)ferrocene]dichloropalladium(II), complex with dichloromethane (9 mg, 0.08 eq, 12.0 μmol) were added. The mixture was capped, purged with argon, then injected with degassed 4:1 Dioxane:H₂O (1.0 mL, 0.1 M), and was heated at 80° C. for 12 h. After this time, the mixture was cooled to room temperature, diluted with ethyl acetate (30 mL), and washed with water (10 mL). The aqueous layer was washed with ethyl acetate (20 mL×3). The combined organic layer was washed with brine (20 mL×2), dried over sodium sulfate, and evaporated in vacuo to obtain the crude product. The crude product was dry-loaded onto silica gel and purified on a 12 g silica gel column (0-20% MeOH/DCM) to afford 4-(4-(4-(4-fluorophenyl)piperazin-1-yl)pyrido[3,2-d]pyrimidin-6-yl)pyridin-2-amine, 24, (43 mg, 109 μmol, 75%) as an orange solid. LCMS [M+H]⁺ 402. 1H NMR (500 MHz, DMSO) δ 8.56 (s, 1H), 8.31 (d, J=8.8 Hz, 1H), 8.22 (d, J=8.8 Hz, 1H), 8.10 (d, J=5.3 Hz, 1H), 7.20 (d, J=5.4 Hz, 1H), 7.15 (s, 1H), 7.09 (t, J=8.8 Hz, 2H), 7.03 (dd, J=9.3, 4.6 Hz, 2H), 6.18 (s, 2H), 4.63 (s, 4H), 3.36 (t, J=5.1 Hz, 4H). 13C NMR (126 MHz, DMSO) δ 161.20, 159.11, 157.55, 155.68, 155.25, 151.96, 149.47, 148.11, 148.09, 147.22, 146.15, 137.69, 133.32, 125.11, 117.72, 117.66, 115.92, 115.74, 109.85, 105.43, 49.58.

### 4-(4-(4-phenylpiperazin-1-yl)quinolin-6-yl)pyridin-2-amine, 25

Compound 25 was prepared via General Scheme 1 via the two step procedure reported for the preparation of compound 1. The two steps are reported below.

### 6-bromo-4-(4-phenylpiperazin-1-yl)quinoline, 102

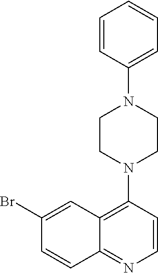 [SMILES: Brc1ccc2nccc(N3CCN(c4ccccc4)CC3)c2c1]

In a 5 mL microwave vial of 1-phenylpiperazine (1.2 g, 1.17 mL, 6 eq., 7.42 mmol) dissolved in isopropyl alcohol (3.0 mL, 0.4 M) was added N,N-Diisopropylethylamine (799 mg, 1.1 mL, 5 eq, 6.19 mmol) and 6-bromo-4-chloroquinoline (300 mg, 1 eq, 1.24 mmol). The reaction mixture was stirred at 100° C. for 12 h. Upon cooling to room temperature, the reaction was poured into water and extracted with EtOAc (20 mL×2), washed with brine, dried over anhydrous sodium sulfate, and evaporated in vacuo to obtain the crude product. The crude product was dry-loaded onto silica gel and purified on a 24 g silica gel column (5-50% EtOAc/Hexanes) to afford 6-bromo-4-(4-phenylpiperazin-1-yl)quinoline, 102, (350 mg, 0.95 mmol, 77%). LCMS [M+H]⁺ 369. 1H NMR (500 MHz, CDCl3) δ 8.75 (d, J=5.0 Hz, 1H), 8.21 (d, J=2.2 Hz, 1H), 7.97 (d, J=8.9 Hz, 1H), 7.75 (dd, J=9.0, 2.3 Hz, 1H), 7.39-7.28 (m, 2H), 7.09-6.99 (m, 2H), 6.97-6.88 (m, 2H), 3.53-3.44 (m, 4H), 3.40 (dd, J=6.3, 3.4 Hz, 4H). 13C NMR (126 MHz, CDCl3) δ 155.96, 151.17, 151.06, 148.17, 132.64, 131.86, 129.29, 126.03, 124.86, 120.41, 119.63, 116.45, 109.70, 52.25, 49.40.

### 4-(4-(4-phenylpiperazin-1-yl)quinolin-6-yl)pyridin-2-amine, 25

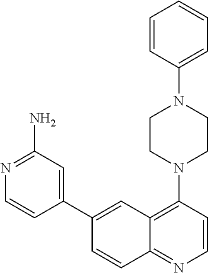 [SMILES: Nc1cc(-c2ccc3nccc(N4CCN(c5ccccc5)CC4)c3c2)ccn1]

In a 5 mL microwave vial, 6-bromo-4-(4-phenylpiperazin-1-yl)quinoline (80 mg, 1 eq, 0.22 mmol), 4-(4,4,5,5-tetramethyl-1,3,2-dioxaborolan-2-yl)pyridin-2-amine (57 mg, 1.2 eq, 0.26 mmol), tripotassium phosphate (180 mg, 4 eq, 0.87 mmol), and [1,1′-Bis(diphenylphosphino)ferrocene]dichloropalladium(II), complex with dichloromethane (14 mg, 0.08 eq, 17.0 μmol) were added. The mixture was capped, purged with argon, then injected with degassed 4:1 Dioxane:H2O (2.0 mL, 0.1 M), and was heated at 80° C. for 6 h. After this time, the mixture was cooled to room temperature, diluted with ethyl acetate (30 mL), and washed with water (10 mL×3). The aqueous layer was washed with ethyl acetate (20 mL×3). The combined organic layer was washed with brine (20 mL), dried over sodium sulfate, and evaporated in vacuo to obtain the crude product. The crude product was dry-loaded onto silica gel and purified on a 12 g silica gel column (0-20% MeOH/DCM) to afford 4-(4-(4-phenylpiperazin-1-yl)quinolin-6-yl)pyridin-2-amine, 25, (38 mg, 101 μmol, 47%) as yellow solid. LCMS [M+H]⁺ 382. 1H NMR (500 MHz, CDCl3) δ 8.79 (d, J=5.1 Hz, 1H), 8.29 (d, J=2.0 Hz, 1H), 8.20 (d, J=8.7 Hz, 1H), 8.12 (d, J=5.6 Hz, 1H), 7.90 (dd, J=8.6, 2.0 Hz, 1H), 7.36-7.28 (m, 2H), 7.04 (d, J=8.1 Hz, 2H), 7.01 (d, J=5.6 Hz, 1H), 6.97 (d, J=5.1 Hz, 1H), 6.94 (t, J=7.3 Hz, 1H), 6.87 (s, 1H), 5.10 (s, 2H), 3.51 (d, J=5.1 Hz, 4H), 3.48 (d, J=5.4 Hz, 4H). 13C NMR (126 MHz, DMSO) δ 160.56, 156.29, 151.42, 150.89, 149.09, 148.76, 147.51, 135.02, 130.45, 129.00, 127.47, 122.75, 121.07, 119.25, 115.66, 110.04, 109.67, 105.07, 51.79, 48.33.

### 4-(4-phenylpiperazin-1-yl)-6-(1H-pyrazol-4-yl)quinoline, 26

Compound 26 was prepared via General Scheme 1 via the two step procedure reported for the preparation of compound 1. The second step is reported below.

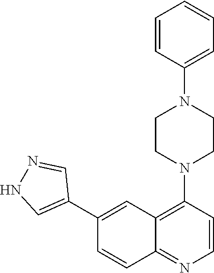 [SMILES: c1ccc(N2CCN(c3ccnc4ccc(-c5cn[nH]c5)cc34)CC2)cc1]

A 5 mL Biotage© microwave vial loaded with tripotassium phosphate (0.22 g, 4 eq, 1.0 mmol), [1,1′-Bis(diphenylphosphino)ferrocene]dichloropalladium(II), complex with dichloromethane (17 mg, 0.08 eq, 20 μmol), 6-bromo-4-(4-phenylpiperazin-1-yl)quinoline (94 mg, 1 eq. 0.26 mmol), and tert-butyl 4-(4,4,5,5-tetramethyl-1,3,2-dioxaborolan-2-yl)-1H-pyrazole-1-carboxylate (90 mg, 1.2 eq, 0.31 mmol). The vial was capped, purged with argon, then injected with degassed 4:1 Dioxane:H2O (1.5 mL, 0.1 M) and heated to 80° C. for 5 h. The reaction was cooled, diluted with DCM:MeOH, filtered through celite, and concentrated. A mixture of DCM:TFA (1:2, 1.5 mL) was added to the crude mixture and stirred at room temperature overnight and basified with 1N NaOH. The mixture was extracted with DCM, washed with 10% NaOH, water, brine, and the organic layer was dried over sodium sulfate, concentrated, and dry-loaded onto silica gel and purified on a 12 g column (DCM/MeOH 0-20%), affording 4-(4-phenylpiperazin-1-yl)-6-(1H-pyrazol-4-yl)quinoline, 26, (12 mg, 35 μmol, 14%) as a red-brown solid. LCMS [M+H]⁺ 356. 1H NMR (500 MHz, DMSO) δ 13.11 (s, 1H), 8.65 (d, J=5.8 Hz, 1H), 8.40 (s, 1H), 8.21 (d, J=1.9 Hz, 1H), 8.11 (dd, J=8.9, 1.8 Hz, 2H), 7.96 (d, J=8.7 Hz, 1H), 7.30-7.24 (m, 2H), 7.13 (d, J=5.8 Hz, 1H), 7.04 (d, J=8.0 Hz, 2H), 6.83 (t, J=7.3 Hz, 1H), 3.66 (s, 4H), 3.50 (t, J=5.0 Hz, 4H).

### 4-(4-(6-(2-aminopyridin-4-yl)quinazolin-4-yl)piperazin-1-yl)phenol, 27

Compound 27 was prepared via General Scheme 1 via the two step procedure reported for the preparation of compound 1. The two steps are reported below.

### 4-(4-(6-bromoquinazolin-4-yl)piperazin-1-yl)phenol, 103

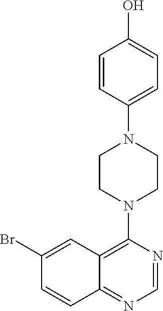 [SMILES: Oc1ccc(N2CCN(c3ncnc4ccc(Br)cc34)CC2)cc1]

In a 5 mL Biotage microwave vial, sodium bicarbonate (45 mg, 1 eq. 0.534 mmol) and 4-(piperazin-1-yl)phenol (95 mg, 1.0 eq, 0.534 mmol), were added with EtOH (1.3 mL, 0.4 M) and stirred for 10 min. 6-bromo-4-chloroquinazoline (130 mg, 1 eq, 0.534 mmol) was added, the vial was sealed, and stirred at 25° C. for 12 h. Upon completion, the reaction was diluted with water and filtered to afford 4-(4-(6-bromoquinazolin-4-yl)piperazin-1-yl)phenol, 103, (0.158 g, 0.410 mmol, 77%) as a tan solid. LCMS [M+H]⁺ 386. 1H NMR (500 MHz, CDCl3) δ 8.54 (t, J=4.3 Hz, 1H), 8.29 (s, 1H), 7.91 (t, J=4.1 Hz, 1H), 7.67 (d, J=4.9 Hz, 2H), 7.25 (d, J=4.8 Hz, 1H), 6.73 (s, 1H), 6.64 (dt, J=8.6, 4.2 Hz, 2H), 3.84 (s, 4H), 3.09 (p, J=4.5 Hz, 4H).

### 4-(4-(6-(2-aminopyridin-4-yl)quinazolin-4-yl)piperazin-1-yl)phenol, 27

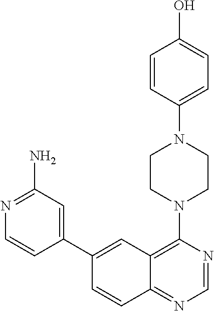 [SMILES: Nc1cc(-c2ccc3ncnc(N4CCN(c5ccc(O)cc5)CC4)c3c2)ccn1]

In a 5 mL microwave vial, 4-(4-(6-chloroquinazolin-4-yl)piperazin-1-yl)phenol (50 mg, 1 eq, 0.15 mmol), 4-(4,4,5,5-tetramethyl-1,3,2-dioxaborolan-2-yl)pyridin-2-amine (39 mg, 1.2 eq, 0.18 mmol), tripotassium phosphate (120 mg, 4 eq, 0.59 mmol), and [1,1′-Bis(diphenylphosphino)ferrocene]dichloropalladium(II), complex with dichloromethane (9 mg, 0.08 eq, 12 μmol) were added. The mixture was capped, purged with argon, then injected with degassed 4:1 Dioxane:H₂O (1.0 mL, 0.1 M), and was heated at 80° C. for 12 h. After this time, the mixture was cooled to room temperature, diluted with ethyl acetate (30 mL), and washed with water (10 mL×3). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (0-10% MeOH/DCM) to afford 4-(4-(6-(2-aminopyridin-4-yl)quinazolin-4-yl)piperazin-1-yl)phenol, 27, (24 mg, 60 μmol, 41%) as a light brown solid. LCMS [M+H]⁺ 399. 1H NMR (500 MHz, DMSO) δ 8.89 (s, 1H), 8.67 (s, 1H), 8.18 (d, J=2.1 Hz, 1H), 8.09 (dd, J=8.7, 2.0 Hz, 1H), 8.03 (d, J=5.3 Hz, 1H), 7.92 (d, J=8.7 Hz, 1H), 6.92 (dd, J=5.3, 1.7 Hz, 1H), 6.90-6.83 (m, 2H), 6.82 (dd, J=1.7, 0.7 Hz, 1H), 6.72-6.65 (m, 2H), 6.06 (s, 2H), 3.94 (t, J=5.0 Hz, 4H), 3.20 (t, J=5.1 Hz, 4H). 13C NMR (126 MHz, DMSO) δ 163.80, 160.55, 154.03, 151.46, 151.27, 148.78, 147.09, 143.89, 135.31, 131.10, 128.85, 122.69, 118.15, 115.89, 115.53, 110.05, 105.16, 50.07, 49.33.

### 4-(4-(6-(2-aminopyridin-4-yl)pyrido[3,2-d]pyrimidin-4-yl)piperazin-1-yl)phenol, 28

Compound 28 was prepared via General Scheme 1 via the two step procedure reported for the preparation of compound 1. The two steps are reported below.

### 4-(4-(6-chloropyrido[3,2-d]pyrimidin-4-yl)piperazin-1-yl)phenol, 104

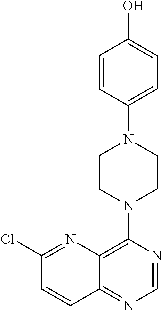 [SMILES: Oc1ccc(N2CCN(c3ncnc4ccc(Cl)nc34)CC2)cc1]

In a 20 mL Biotage microwave vial, sodium bicarbonate (189 mg, 1 eq, 2.25 mmol) and 4-(piperazin-1-yl)phenol (401 mg, 1.0 eq, 2.25 mmol), were added with EtOH (5.0 mL, 0.4 M) and stirred for 10 min. 4,6-dichloropyrido[3,2-d]pyrimidine (450 mg, 1 eq, 2.25 mmol) was added, the vial was sealed, and stirred at 25° C. for 2 days. Upon completion, the reaction was diluted with water and filtered to afford 4-(4-(6-chloropyrido[3,2-d]pyrimidin-4-yl)piperazin-1-yl)phenol, 104, (0.429 g, 1.26 mmol, 56%) as a tan solid. LCMS [M+H]+ 342. 1H NMR (500 MHz, DMSO) δ 8.86 (s, 1H), 8.57 (s, 1H), 8.18 (d, J=8.8 Hz, 1H), 7.88 (d, J=8.8 Hz, 1H), 6.89-6.81 (m, 2H), 6.73-6.61 (m, 2H), 4.47 (s, 4H), 3.24-3.02 (m, 4H). 13C NMR (126 MHz, DMSO) δ 157.60, 154.96, 151.22, 146.46, 144.90, 143.78, 140.00, 132.25, 128.95, 118.07, 115.48, 50.24.

### 4-(4-(6-(2-aminopyridin-4-yl)pyrido[3,2-d]pyrimidin-4-yl)piperazin-1-yl)phenol, 28

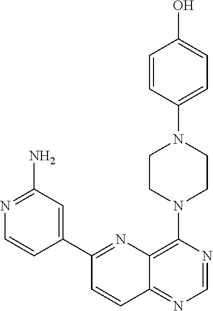 [SMILES: Nc1cc(-c2ccc3ncnc(N4CCN(c5ccc(O)cc5)CC4)c3n2)ccn1]

In a 20 mL microwave vial, 4-(4-(6-chloropyrido[3,2-d]pyrimidin-4-yl)piperazin-1-yl)phenol (429 mg, 1 eq, 1.26 mmol), 4-(4,4,5,5-tetramethyl-1,3,2-dioxaborolan-2-yl)pyridin-2-amine (331 mg, 1.2 eq, 1.51 mmol), tripotassium phosphate (1.07 g, 4 eq, 5.02 mmol), and [1,1′-Bis(diphenylphosphino)ferrocene]dichloropalladium(II), complex with dichloromethane (82 mg, 0.08 eq, 100 μmol) were added. The mixture was capped, purged with argon, then injected with degassed 4:1 Dioxane:H₂O (10 mL, 0.1 M), and was heated at 80° C. for 5 h. After this time, the mixture was cooled to room temperature, diluted with ethyl acetate (30 mL), and washed with water (10 mL×3). The aqueous layer was washed with ethyl acetate (20 mL×3). The combined organic layer was washed with brine (20 mL), dried over sodium sulfate, and evaporated in vacuo to obtain the crude product. The crude product was dry-loaded onto silica gel and purified on a 24 g silica gel column (0-20% MeOH/DCM) to afford 4-(4-(6-(2-aminopyridin-4-yl)pyrido[3,2-d]pyrimidin-4-yl)piperazin-1-yl)phenol, 28, (200 mg, 501 μmol, 40%) as a tan solid. LCMS [M+H]⁺ 400. 1H NMR (500 MHz, DMSO) δ 8.90 (s, 1H), 8.56 (s, 1H), 8.31 (d, J=8.8 Hz, 1H), 8.22 (d, J=8.8 Hz, 1H), 8.09 (d, J=5.3 Hz, 1H), 7.19 (dd, J=5.4, 1.6 Hz, 1H), 7.15 (dd, J=1.7, 0.8 Hz, 1H), 6.91-6.85 (m, 2H), 6.72-6.65 (m, 2H), 6.17 (s, 2H), 4.61 (s, 5H), 3.22 (t, J=4.8 Hz, 4H).

### Step 1, General Scheme 2

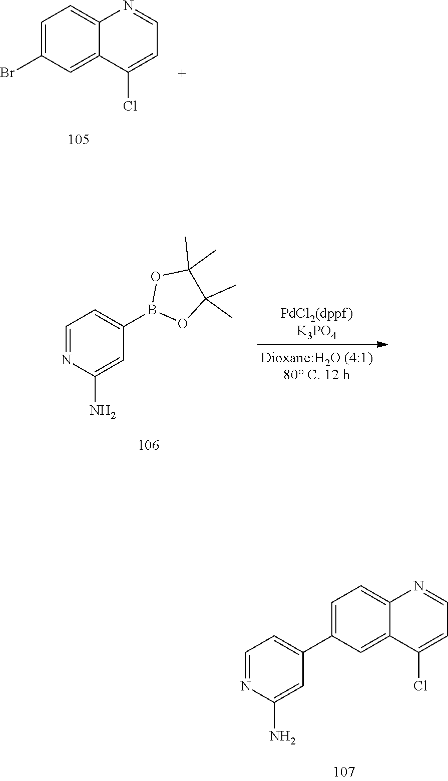 [SMILES: Clc1ccnc2ccc(Br)cc12 ; CC1(C)OB(c2ccnc(N)c2)OC1(C)C ; Nc1cc(-c2ccc3nccc(Cl)c3c2)ccn1]

### 4-(4-chloroquinolin-6-yl)pyridin-2-amine, 107

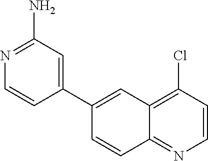 [SMILES: Nc1cc(-c2ccc3nccc(Cl)c3c2)ccn1]

A 100 mL RBF was loaded with tripotassium phosphate (1.05 g, 1.5 eq, 4.95 mmol), [1,1′-Bis(diphenylphosphino)ferrocene]dichloropalladium(II), complex with dichloromethane (270 mg, 0.1 eq, 330 μmol), 6-bromo-4-chloroquinoline (800 mg, 1 eq, 3.30 mmol), and 4-(4,4,5,5-tetramethyl-1,3,2-dioxaborolan-2-yl)pyridin-2-amine (799 mg, 1.1 eq, 3.63 mmol). The mixture was injected with degassed 4:1 Dioxane:H₂O (16 mL, 0.2 M), purged with argon for 10 minutes, and refluxed at 80° C. for 12 h. The reaction was cooled, diluted with DCM:MeOH, filtered through celite, concentrated, dry-loaded onto silica gel and purified on 24 g column (DCM/MeOH 0-8%), affording 4-(4-chloroquinolin-6-yl)pyridin-2-amine, 107, (428 mg, 1.67 mmol, 51%). LCMS [M+H]⁺ 256. 1H NMR (500 MHz, CDCl3) δ 8.82 (d, J=4.7 Hz, 1H), 8.44 (d, J=1.9 Hz, 1H), 8.26-8.10 (m, 2H), 7.99 (dd, J=8.8, 2.0 Hz, 1H), 7.55 (d, J=4.7 Hz, 1H), 7.04 (d, J=5.0 Hz, 1H), 6.90 (s, 1H), 4.95 (s, 2H).

### Step 2, General Scheme 2

### 4-(4-(4-phenylpiperazin-1-yl)quinolin-6-yl)pyridin-2-amine, 29

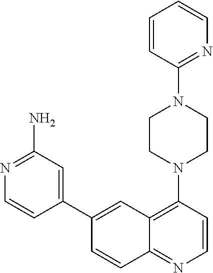 [SMILES: Nc1cc(-c2ccc3nccc(N4CCN(c5ccccn5)CC4)c3c2)ccn1]

In a 5 mL microwave vial of 1-(pyridin-2-yl)piperazine (0.11 g, 0.10 mL, 6 eq, 0.68 mmol) dissolved in isopropyl alcohol (0.8 mL, 0.1 M) was added N,N-Diisopropylethylamine (73 mg, 99 μL, 5 eq, 0.57 mmol) and 4-(4-chloroquinolin-6-yl)pyridin-2-amine (29 mg, 1 eq, 0.11 mmol). The reaction mixture was stirred at 100° C. for 12 h. Upon cooling to room temperature, the reaction was poured into water (10 mL) and extracted with EtOAc (40 mL), washed with brine (20 mL×2), dried over anhydrous sodium sulfate, and evaporated in vacuo to obtain the crude product. The crude product was dry-loaded onto silica gel and purified on a 12 g silica gel column (0-20% MeOH/DCM) to afford 4-(4-(4-phenylpiperazin-1-yl)quinolin-6-yl)pyridin-2-amine, 29, (33 mg, 0.11 mmol, 77%) as a tan solid. LCMS [M+H]⁺ 383. 1H NMR (500 MHz, DMSO) δ 8.74 (d, J=4.9 Hz, 1H), 8.30 (d, J=2.1 Hz, 1H), 8.17 (dd, J=5.1, 1.9 Hz, 1H), 8.07 (d, J=8.7 Hz, 1H), 8.03 (d, J=5.3 Hz, 1H), 7.99 (dd, J=8.8, 2.0 Hz, 1H), 7.60 (ddd, J=8.9, 7.1, 2.0 Hz, 1H), 7.10 (d, J=5.0 Hz, 1H), 6.97-6.89 (m, 2H), 6.86 (d, J=1.7 Hz, 1H), 6.71 (dd, J=7.1, 4.9 Hz, 1H), 6.06 (s, 2H), 3.81 (d, J=5.5 Hz, 4H), 3.35 (d, J=5.0 Hz, 4H).

### 4-(4-(4-(4-fluorophenyl)piperazin-1-yl)quinolin-6-yl)pyridin-2-amine, 30

Compound 30 was prepared via General Scheme 2 via the two step procedure reported for the preparation of compound 29. The second step is reported below.

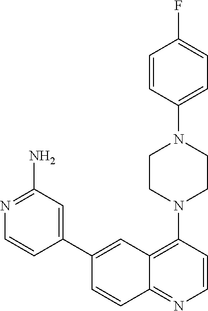 [SMILES: Nc1cc(-c2ccc3nccc(N4CCN(c5ccc(F)cc5)CC4)c3c2)ccn1]

Prepared in an analogous manner to Step 2, General Scheme 2 as seen for 4-(4-(4-phenylpiperazin-1-yl)quinolin-6-yl)pyridin-2-amine, 29. 4-(4-(4-(4-fluorophenyl)piperazin-1-yl)quinolin-6-yl)pyridin-2-amine, 30, (13 mg, 0.16 mmol, 21%) as a tan solid. LCMS [M+H]⁺ 400. 1H NMR (500 MHz, CDCl3) δ 8.78 (d, J=5.0 Hz, 1H), 8.26 (d, J=2.1 Hz, 1H), 8.15 (d, J=8.4 Hz, 2H), 7.88 (dd, J=8.7, 2.0 Hz, 1H), 6.99 (dddd, J=17.3, 12.6, 7.5, 3.7 Hz, 6H), 6.82 (s, 1H), 4.82 (s, 2H), 3.48-3.37 (m, 8H). 13C NMR (101 MHz, CDCl3) δ 158.85, 158.75, 157.11, 156.37, 151.39, 150.25, 149.60, 148.07, 147.69, 147.67, 135.59, 130.79, 127.99, 123.48, 121.92, 118.28, 118.21, 115.83, 115.61, 112.71, 109.49, 106.64, 52.35, 50.40.

### 4-(4-(4-(4-fluorophenyl)piperazin-1-yl)quinazolin-6-yl)pyridin-2-amine, 31

Compound 31 was prepared via General Scheme 1 via the two step procedure reported for the preparation of compound 1. The two steps are reported below.

### 6-bromo-4-(4-(4-fluorophenyl)piperazin-1-yl)quinazoline, 108

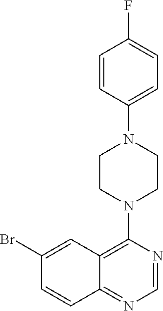 [SMILES: Fc1ccc(N2CCN(c3ncnc4ccc(Br)cc34)CC2)cc1]

In a 5 mL Biotage microwave vial, 1-(4-fluorophenyl)piperazine (96 mg, 96 μL, 1.0 eq, 534 μmol) and sodium bicarbonate (45 mg, 1 eq, 534 μmol) were dissolved in EtOH (1.3 mL, 0.4 M), and stirred for 10 mins. 6-bromo-4-chloroquinazoline (130 mg, 1 eq, 534 μmol) was added, vial capped, and stirred at 25° C. for 12 hrs. After this time, the mixture was cooled to room temperature, diluted with water and filtered to afford 6-bromo-4-(4-(4-fluorophenyl)piperazin-1-yl)quinazoline, 108 (169 mg, 437 μmol, 82%) as a tan solid. LCMS [M+H]⁺ 388. 1H NMR (500 MHz, CDCl₃) δ 8.75 (s, 1H), 8.07 (d, J=2.1 Hz, 1H), 7.94-7.78 (m, 2H), 7.07-6.97 (m, 2H), 6.97-6.90 (m, 2H), 3.99 (t, J=5.1 Hz, 4H), 3.51-3.24 (m, 4H).

### 4-(4-(4-(4-fluorophenyl)piperazin-1-yl)quinazolin-6-yl)pyridin-2-amine, 31

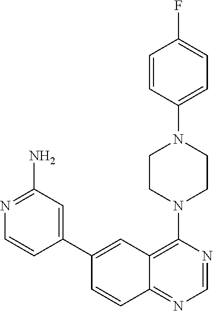 [SMILES: Nc1cc(-c2ccc3ncnc(N4CCN(c5ccc(F)cc5)CC4)c3c2)ccn1]

In a 5 mL microwave vial, 6-chloro-4-(4-(4-fluorophenyl)piperazin-1-yl)quinazoline (60 mg, 1 eq, 0.18 mmol), 4-(4,4,5,5-tetramethyl-1,3,2-dioxaborolan-2-yl)pyridin-2-amine (46 mg, 1.2 eq, 0.21 mmol), tripotassium phosphate (0.15 mg, 4 eq, 0.7 mmol), and [1,1′-Bis(diphenylphosphino)ferrocene]dichloropalladium(II), complex with dichloromethane (11 mg, 0.08 eq, 14 μmol) were added. The mixture was capped, purged with argon, then injected with degassed 4:1 Dioxane:H₂O (1.5 mL, 0.1 M), and was heated at 80° C. for 6 h. After this time, the mixture was cooled to room temperature, diluted with ethyl acetate (30 mL), and washed with water (10 mL×3). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (0-10% MeOH/DCM) to afford 4-(4-(4-(4-fluorophenyl)piperazin-1-yl)quinazolin-6-yl)pyridin-2-amine, 31, (28 mg, 0.18 mmol, 40%) as a yellow solid. LCMS [M+H]⁺ 401. 1H NMR (500 MHz, CDCl3) δ 8.79 (s, 1H), 8.13 (d, J=5.5 Hz, 1H), 8.11 (d, J=2.0 Hz, 1H), 8.00 (d, J=8.7 Hz, 1H), 7.96 (dd, J=8.7, 1.9 Hz, 1H), 7.04-6.97 (m, 2H), 6.97-6.91 (m, 3H), 6.81 (d, J=1.6 Hz, 1H), 4.98 (s, 2H), 4.00 (t, J=5.0 Hz, 4H), 3.34 (t, J=5.0 Hz, 4H). 13C NMR (101 MHz, CDCl3) δ 164.94, 159.01, 158.91, 156.53, 154.72, 152.23, 149.75, 148.41, 147.77, 147.74, 135.99, 131.46, 129.74, 123.26, 118.43, 118.35, 116.86, 116.00, 115.78, 112.67, 106.60, 50.40, 50.01.

### Step 1, General Scheme 3

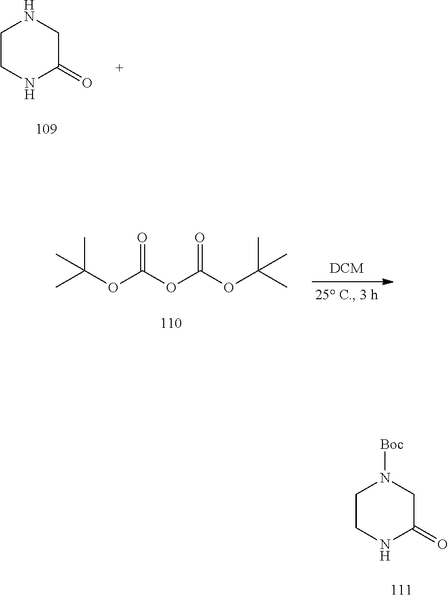

### tert-butyl 3-oxopiperazine-1-carboxylate, 111

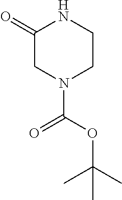

In a 100 mL RBF was added piperazin-2-one (600 mg, 1.0 eq, 5.99 mmol) in DCM (40 mL, 0.15 M). Di-tert-butyl dicarbonate (1.44 g, 1.41 mL, 1.1 eq, 6.59 mmol) was added via syringe and stirred at room temperature for 3 h. Upon completion, reaction mixture was diluted with DCM (30 mL) and washed with water (50 mL). The aqueous layer was washed with DCM (50×2 mL). The combined organic layer was washed with brine (30×2 mL), dried over sodium sulfate, and evaporated in vacuo to obtain tert-butyl 3-oxopiperazine-1-carboxylate, 111, (1.2 g, 5.5 mmol, 92%) as a white solid. LCMS [M+H]⁺ 101. 1H NMR (500 MHz, CDCl3) δ 6.68 (s, 1H), 4.08 (s, 2H), 3.62 (t, J=5.4 Hz, 2H), 3.38 (td, J=5.3, 2.6 Hz, 2H), 1.47 (s, 9H).

### Step 2, General Scheme 3

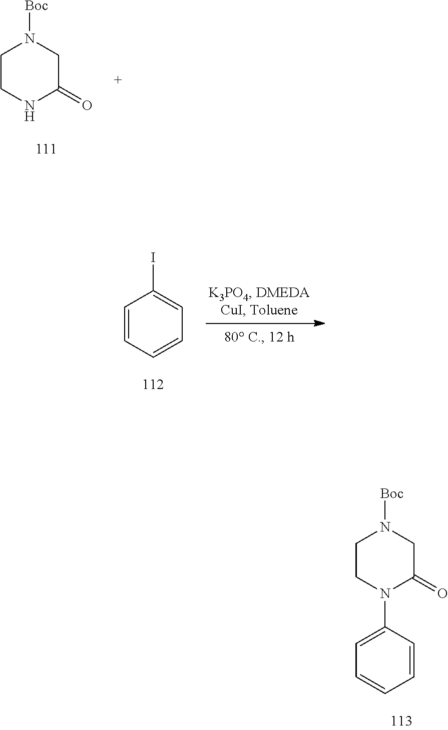 [SMILES: CC(C)(C)OC(=O)N1CCNC(=O)C1 ; Ic1ccccc1 ; CC(C)(C)OC(=O)N1CCN(c2ccccc2)C(=O)C1]

### tert-butyl 3-oxo-4-phenylpiperazine-1-carboxylate, 113

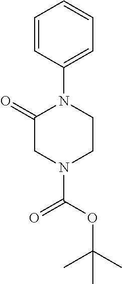 [SMILES: CC(C)(C)OC(=O)N1CCN(c2ccccc2)C(=O)C1]

To a solution of tert-butyl 3-oxopiperazine-1-carboxylate (300 mg, 1.2 eq, 1.50 mmol), iodobenzene (255 mg, 140 μL, 1 eq, 1.25 mmol), N,N′-dimethylethylenediamine (11 mg, 14 μL, 0.1 eq, 125 μmol), and tripotassium phosphate (530 mg, 2 eq, 2.50 mmol) in toluene (2.3 mL, 0.6 M) was added Copper Iodide (11 mg, 0.05 eq, 62 μmol). The reaction mixture was heated to 80° C. for 12 hours. The reaction mixture was cooled to room temperature, diluted with DCM, and filtered through a plug of silica using 40% EtOAc/Hexane as eluent to afford tert-butyl 3-oxo-4-phenylpiperazine-1-carboxylate, 113, (191 mg, 0.693 mmol, 56%) as a white solid. LCMS [M+H]⁺ 277. 1H NMR (500 MHz, CDCl3) δ 7.42 (dd, J=8.7, 7.1 Hz, 2H), 7.32-7.26 (m, 311), 4.26 (s, 2H), 3.79 (dd, J=6.6, 3.7 Hz, 2H), 3.74 (dd, J=6.9, 4.4 Hz, 2H), 1.50 (s, 9H).

### Step 3, General Scheme 3

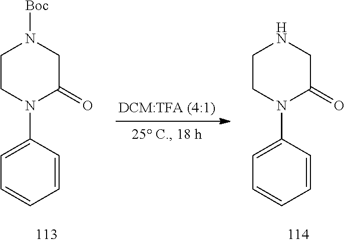 [SMILES: CC(C)(C)OC(=O)N1CCN(c2ccccc2)C(=O)C1 ; O=C1CNCCN1c1ccccc1]

### 1-phenylpiperazin-2-one, 114

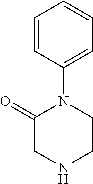 [SMILES: O=C1CNCCN1c1ccccc1]

A mixture of DCM:TFA (1:4, 14.6 mL) was added to the crude mixture and stirred at room temperature overnight. Upon completion, the remaining TFA was evaporated off in vacuo to afford 1-phenylpiperazin-2-one (128 mg, 662 μmol, 34%) as a TFA salt. LCMS [M+H]⁺ 195. 1H NMR (500 MHz, CDCl3) δ 7.25 (d, J=2.9 Hz, 1H), 6.95 (tq, J=6.9, 2.3 Hz, 2H), 6.84 (dddd, J=10.0, 8.4, 3.6, 2.1 Hz, 2H), 3.48 (d, J=5.3 Hz, 2H), 3.46 (d, J=4.5 Hz, 2H), 3.12 (dd, J=6.6, 4.3 Hz, 2H).

### Step 4, General Scheme 3

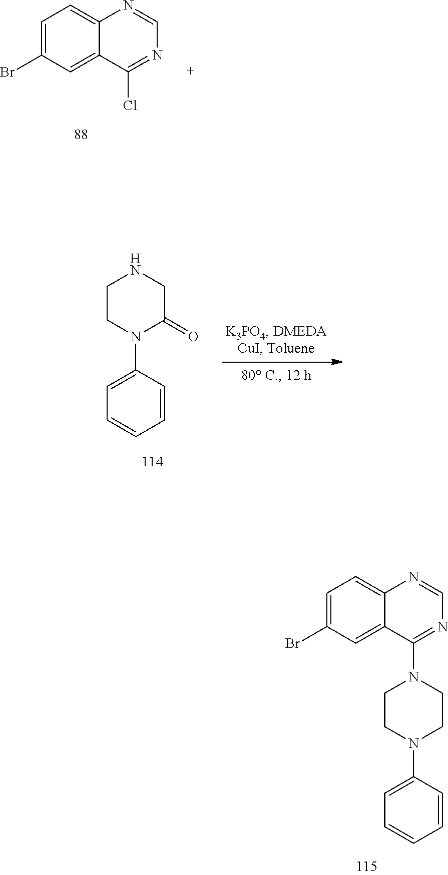 [SMILES: Clc1ncnc2ccc(Br)cc12 ; O=C1CNCCN1c1ccccc1 ; Brc1ccc2ncnc(N3CCN(c4ccccc4)CC3)c2c1]

### 4-(6-bromoquinazolin-4-yl)-1-phenylpiperazin-2-one, 115

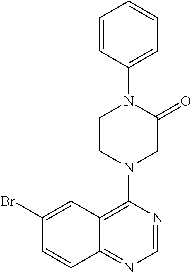 [SMILES: O=C1CN(c2ncnc3ccc(Br)cc23)CCN1c1ccccc1]

To a solution of 1-phenylpiperazin-2-one, trifluoroacetic acid (100 mg, 1.2 eq, 345 mol), 6-bromo-4-chloroquinazoline (69 mg, 1.0 eq, 287 μmol), N,N′-dimethylethylenediamine (2.5 mg, 3 L, 0.1 eq, 28 mol), and K3PO4 (122 mg, 2 eq, 574 μmol) in Toluene (0.8 mL, 0.4 M) was added Copper Iodide (2. mg, 0.05 eq, 14 μmol). The reaction mixture was heated to 80° C. for 12 hours. The reaction mixture was cooled to room temp, diluted with DCM, and filtered through a plug of silica using 5% MeOH:DCM as eluent. The filtrate was dry loaded onto silica gel and purified on a 12 g column (DCM/MeOH 0-8%), affording 4-(6-bromoquinazolin-4-yl)-1-phenylpiperazin-2-one, 115, (76 mg, 198 μmol, 69%). LCMS [M+H]⁺ 384. 1H NMR (500 MHz, CDCl3) δ 8.82 (s, 1H), 8.17 (s, 2H), 7.93 (s, 1H), 7.49-7.39 (m, 2H), 7.38-7.28 (m, 3H), 4.73 (s, 2H), 4.31 (s, 2H), 4.00 (t, J=5.1 Hz, 2H).

### 4-(6-(1H-pyrazol-4-yl)quinazolin-4-yl)-1-phenylpiperazin-2-one, 32

Compound 32 was prepared via General Scheme 3 via the five step procedure. The fifth step is reported below.

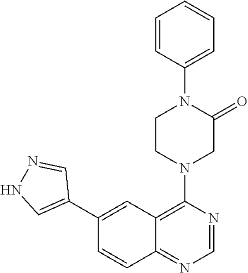 [SMILES: O=C1CN(c2ncnc3ccc(-c4cn[nH]c4)cc23)CCN1c1ccccc1]

A 5 mL Biotage© microwave vial loaded with tripotassium phosphate (96 mg, 4 eq, 455 μmol), [1,1′-Bis(diphenylphosphino)ferrocene]dichloropalladium(II), complex with dichloromethane (7.4 mg, 0.08 eq, 9.09 μmol), 4-(6-chloroquinazolin-4-yl)-1-phenylpiperazin-2-one (38 mg, 1 eq, 114 μmol), and tert-butyl 4-(4,4,5,5-tetramethyl-1,3,2-dioxaborolan-2-yl)-1H-pyrazole-1-carboxylate (40 mg, 1.2 eq, 136 μmol). The vial was capped, purged with argon, then injected with degassed 4:1 Dioxane:H2O (1.5 mL, 76 mM) and heated to 80° C. for 12 h. The reaction was cooled, diluted with DCM:MeOH, filtered through celite, and concentrated. A mixture of DCM:TFA (1:2, 1.5 mL) was added to the crude mixture and stirred at room temperature overnight and basified with 1N NaOH. The mixture was extracted with DCM, washed with 10% NaOH, water, brine, and the organic layer was dried over sodium sulfate, concentrated, and dry-loaded onto silica gel and purified on a 12 g column (DCM/MeOH 0-10%), affording 4-(6-(1H-pyrazol-4-yl)quinazolin-4-yl)-1-phenylpiperazin-2-one, 32 (22 mg, 59 μmol, 52%) as a tan solid. LCMS [M+H]⁺ 371. 1H NMR (500 MHz, DMSO) δ 13.08 (s, 1H), 8.63 (s, 1H), 8.43 (s, 1H), 8.20 (d, J=1.9 Hz, 1H), 8.17-8.12 (m, 2H), 7.84 (d, J=8.6 Hz, 1H), 7.42 (d, J=5.3 Hz, 4H), 7.28 (ddd, J=10.2, 5.8, 3.1 Hz, 1H), 4.55 (s, 2H), 4.24 (t, J=5.2 Hz, 2H), 3.97 (t, J=5.2 Hz, 2H). 13C NMR (126 MHz, CD3OD_SPE) δ 176.88, 167.02, 163.03, 152.74, 149.59, 141.63, 131.48, 131.15, 128.99, 127.63, 127.17, 125.83, 121.19, 120.01, 116.46, 52.65, 49.01, 46.46.

### 4-(4-(4-(2-fluorophenyl)piperazin-1-yl)quinolin-6-yl)pyridin-2-amine, 33

Compound 33 was prepared via General Scheme 2 via the two step procedure reported for the preparation of compound 29. The second step is reported below.

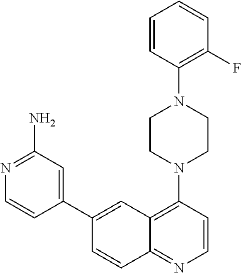 [SMILES: Nc1cc(-c2ccc3nccc(N4CCN(c5ccccc5F)CC4)c3c2)ccn1]

Prepared in analogous manner to Step 2, General Scheme 2 as used for compound 29. 4-(4-(4-(2-fluorophenyl)piperazin-1-yl)quinolin-6-yl)pyridin-2-amine, 33, (31 mg, 0.16 mmol, 49%) as a tan solid. LCMS [M+H]⁺ 400. 1H NMR (500 MHz, CDCl3) δ 8.79 (d, J=5.1 Hz, 1H), 8.28 (d, J=2.0 Hz, 1H), 8.19 (d, J=8.7 Hz, 1H), 8.14 (d, J=5.4 Hz, 1H), 7.89 (dd, J=8.7, 2.0 Hz, 1H), 7.21-7.05 (m, 3H), 7.04-6.99 (m, 2H), 6.98 (d, J=5.1 Hz, 1H), 6.86 (s, 1H), 4.99 (s, 2H), 3.49 (dd, J=5.8, 3.3 Hz, 4H), 3.45-3.39 (m, 4H). 13C NMR (126 MHz, CDCl3) δ 158.88, 157.23, 156.81, 154.85, 151.45, 150.21, 149.62, 148.34, 139.75, 139.68, 135.59, 130.81, 127.98, 124.62, 124.59, 123.50, 123.12, 123.06, 121.98, 119.22, 119.20, 116.41, 116.25, 112.84, 109.55, 106.59, 52.46, 50.64.

### 4-(4-(4-(2,4-dimethylphenyl)piperazin-1-yl)quinolin-6-yl)pyridin-2-amine, 34

Compound 34 was prepared via General Scheme 2 via the two step procedure reported for the preparation of compound 29. The second step is reported below.

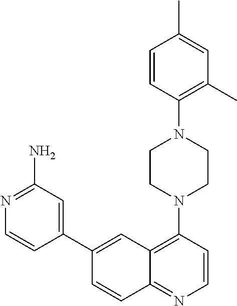 [SMILES: Cc1ccc(N2CCN(c3ccnc4ccc(-c5ccnc(N)c5)cc34)CC2)c(C)c1]

Prepared in analogous manner to Step 2, General Scheme 2 as used for compound 29. 4-(4-(4-(2,4-dimethylphenyl)piperazin-1-yl)quinolin-6-yl)pyridin-2-amine, 34, (33 mg, 80 mol, 41%) as a tan solid. LCMS [M+H]⁺ 410. 1H NMR (400 MHz, CDCl₃) δ 8.77 (d, J=5.1 Hz, 1H), 8.30 (d, J=2.1 Hz, 1H), 8.21 (d, J=8.7 Hz, 1H), 8.11 (d, J=5.7 Hz, 1H), 7.89 (dd, J=8.8, 2.1 Hz, 1H), 7.10-7.03 (m, 3H), 7.03-6.99 (m, 1H), 6.97 (d, J=5.2 Hz, 1H), 6.90 (d, J=1.6 Hz, 1H), 5.21 (s, 2H), 3.48 (dt, J=5.4, 2.3 Hz, 4H), 3.24-3.15 (m, 4H), 2.33 (s, 3H), 2.30 (s, 3H).

### 4-(4-(6,7-dimethoxy-3,4-dihydroisoquinolin-2(1H)-yl)quinazolin-6-yl)pyridin-2-amine, 35

Compound 35 was prepared via General Scheme 1 via the two step procedure reported for the preparation of compound 1. The second step is reported below.

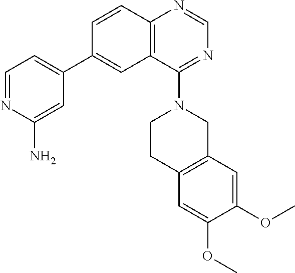 [SMILES: COc1cc2c(cc1OC)CN(c1ncnc3ccc(-c4ccnc(N)c4)cc13)CC2]

To a microwave vial potassium phosphate, tribasic (318 mg, 3 eq, 1.50 mmol), PdCl₂(dppf) (36 mg, 0.1 eq, 50 μmol), 4-(4,4,5,5-tetramethyl-1,3,2-dioxaborolan-2-yl)pyridin-2-amine (121 mg, 1.1 eq, 550 mol), and 6-bromo-4-(6,7-dimethoxy-3,4-dihydroisoquinolin-2(1H)-yl)quinazoline (200 mg, 1 eq, 500 μmol) was added and the vial was sealed. The solids were dissolved in 1,4-Dioxane (2.0 mL) and water (0.40 mL), and the reaction mixture was heated to 90° C. where it stirred for 16 h. After time elapsed, the reaction mixture was cooled to room temperature, diluted with ethyl acetate and filtered through a thick celite pad. The filtrate was collected and concentrated via rotary evaporation. The resulting residue was dissolved in 50 mL of ethyl acetate and washed with water (3×50 mL). The organic layer was dried over sodium sulfate, filtered, and concentrated via rotary evaporation. The crude concentrate was purified via column chromatography (0-10% MeOH/DCM) producing 4-(4-(6,7-dimethoxy-3,4-dihydroisoquinolin-2(1H)-yl)quinazolin-6-yl)pyridin-2-amine, 35, (157 mg, 380 μmol, 76%). LCMS: [M+H]⁺ 414. ¹H NMR (400 MHz, MeOD) δ 8.60 (s, 1H), 8.21 (s, 1H), 8.03 (dd, J=16.2, 7.0 Hz, 2H), 7.86 (d, J=8.8 Hz, 1H), 6.92 (d, J=5.4 Hz, 1H), 6.90-6.67 (m, 3H), 4.01 (d, J=5.9 Hz, 2H), 3.70 (d, J=9.4 Hz, 6H), 3.02 (t, J=5.8 Hz, 2H), 2.48 (s, 2H).

### 4-(4-(4-fluorobenzyl)-1,4-diazepan-1-yl)-6-(1H-pyrazol-4-yl)quinazoline, 36

Compound 36 was prepared via General Scheme 1 via the two step procedure reported for the preparation of compound 1. The second step is reported below.

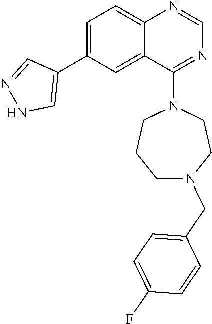 [SMILES: Fc1ccc(CN2CCCN(c3ncnc4ccc(-c5cn[nH]c5)cc34)CC2)cc1]

Potassium acetate (156 mg, 3 eq, 1.59 mmol), PdCl₂(dppf) (38 mg, 0.1 eq, 53 μmol), tert-butyl 4-(4,4,5,5-tetramethyl-1,3,2-dioxaborolan-2-yl)-1H-pyrazole-1-carboxylate (171 mg, 1.1 eq, 583 μmol), 6-bromo-4-(4-(4-fluorobenzyl)-1,4-diazepan-1-yl)quinazoline (220 mg, 1 eq, 530 μmol) were added to a MWV, the solids were degassed then dissolved in degassed solvent 1,4-Dioxane (1.75 mL) and water (350 μL), the solution was heated to 90° C. and stirred using magnetic stirring for 16 h. After time elapsed the solution was diluted with EtOAc and filtered through a pad of celite. the filtrate was then concentrated via rotary evaporation and the residue was dissolved in EtOAc and washed twice with water and once with brine. The organic layer was collected and dried using Sodium Sulfate, which was then concentrated in vacuo, the residue was further purified using flash chromatography (MeOH/DCM 0-20%) resulting in a black sludge. The crude tert-butyl 4-(4-(4-(4-fluorobenzyl)-1,4-diazepan-1-yl)quinazolin-6-yl)-1H-pyrazole-1-carboxylate (66 mg, 1 eq, 0.13 mmol) was suspended in DCM (5 mL) and TFA (75 mg, 51 μL, 5 eq, 0.66 mmol) added. The solution stirred for 2 h at room temperature, after time elapsed the reaction mixture was concentrated via rotary evaporation, the residue was dissolved in ethyl acetate and washed with 2M NaOH solution (2×75 mL). The organic layer was collected, dried with sodium sulfate, filtered and concentrated resulting in 4-(4-(4-fluorobenzyl)-1,4-diazepan-1-yl)-6-(1H-pyrazol-4-yl)quinazoline, 36, (48 mg, 0.12 mmol, 91%). LCMS [M+H]⁺ 403. ¹H NMR (400 MHz, DMSO-D₆) δ 13.06 (s, 1H), 8.39 (s, 1H), 8.18-8.09 (m, 3H), 7.98 (dd, J=8.6, 1.8 Hz, 1H), 7.68 (d, J=8.6 Hz, 1H), 7.27 (ddd, J=8.8, 5.6, 2.6 Hz, 2H), 7.07 (td, J=9.1, 2.5 Hz, 2H), 3.95 (q, J=5.8 Hz, 4H), 3.54 (d, J=3.6 Hz, 2H), 2.86-2.79 (m, 2H), 2.58-2.51 (m, 2H), 2.04-1.93 (m, 2H).

### 4-(4-(4-(pyridin-3-yl)piperazin-1-yl)quinazolin-6-yl)pyridin-2-amine, 37

Compound 37 was prepared via General Scheme 1 via the two step procedure reported for the preparation of compound 1. The second step is reported below.

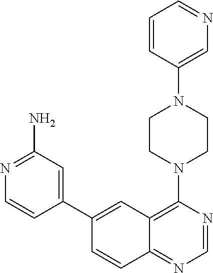 [SMILES: Nc1cc(-c2ccc3ncnc(N4CCN(c5cccnc5)CC4)c3c2)ccn1]

In a 5 mL microwave vial, 6-chloro-4-(4-(pyridin-3-yl)piperazin-1-yl)quinazoline (30 mg, 1 eq, 93 μmol), 4-(4,4,5,5-tetramethyl-1,3,2-dioxaborolan-2-yl)pyridin-2-amine (24 mg, 1.2 eq, 112 μmol), tripotassium phosphate (79 mg, 4 eq, 374 μmol), and [1,1′-Bis(diphenylphosphino)ferrocene]dichloropalladium(II), complex with dichloromethane (6 mg, 0.08 eq, 7.5 μmol) were added. The mixture was capped, purged with argon, then injected with degassed 4:1 Dioxane:H2O (1.4 mL, 67 mM), and was heated at 80° C. for 12 h. After this time, the mixture was cooled to room temperature, diluted with ethyl acetate (30 mL), and washed with water (10 mL×2). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (0-10% MeOH/DCM) to afford 4-(4-(4-(pyridin-3-yl)piperazin-1-yl)quinazolin-6-yl)pyridin-2-amine, 37, (17 mg, 93 μmol, 49%) as a yellow solid. LCMS [M+H]⁺ 384. 1H NMR (500 MHz, CDCl3) δ 8.80 (s, 1H), 8.39 (d, J=2.9 Hz, 1H), 8.18-8.13 (m, 2H), 8.12 (d, J=2.0 Hz, 1H), 8.01 (d, J=8.7 Hz, 1H), 7.97 (dd, J=8.7, 1.9 Hz, 1H), 7.26-7.24 (m, 1H), 7.22 (dd, J=8.4, 4.5 Hz, 1H), 6.94 (dd, J=5.5, 1.6 Hz, 1H), 6.81 (d, J=1.6 Hz, 1H), 4.96 (s, 2H), 4.02 (t, J=5.1 Hz, 4H), 3.51-3.43 (m, 4H). 13C NMR (126 MHz, CDCl3) δ 164.78, 158.72, 154.58, 152.08, 149.73, 147.89, 146.63, 141.31, 138.67, 135.90, 131.41, 129.68, 123.65, 123.08, 122.73, 116.70, 112.51, 106.61, 77.28, 77.02, 76.77, 49.57, 48.46.

### 4-(6-(2-aminopyridin-4-yl)quinazolin-4-yl)-1-phenylpiperazin-2-one, 38

Compound 38 was prepared via General Scheme 3 as shown for the preparation of compound 32. The fifth step is reported below.

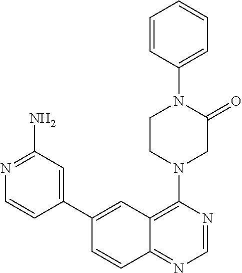 [SMILES: Nc1cc(-c2ccc3ncnc(N4CCN(c5ccccc5)C(=O)C4)c3c2)ccn1]

Prepared in analogous manner to Step 5, General Scheme 3 as used for compound 32. 4-(6-(2-aminopyridin-4-yl)quinazolin-4-yl)-1-phenylpiperazin-2-one, 38, (18 mg, 46 mol, 41%) as a tan solid. LCMS [M+H]⁺ 397. 1H NMR (500 MHz, MeOD) δ 7.97 (s, 1H), 7.63 (d, J=2.0 Hz, 1H), 7.44 (dd, J=8.8, 2.0 Hz, 1H), 7.30 (d, J=5.5 Hz, 1H), 7.24 (d, J=8.7 Hz, 1H), 6.74 (t, J=7.8 Hz, 2H), 6.70-6.65 (m, 2H), 6.65-6.59 (m, 1H), 6.31 (dd, J=5.5, 1.7 Hz, 1H), 6.26 (d, J=1.7 Hz, 1H), 4.05 (s, 2H), 3.68 (t, J=5.3 Hz, 2H), 3.30 (t, J=5.3 Hz, 2H). 13C NMR (126 MHz, CD3OD_SPE) δ 166.74, 163.13, 160.16, 153.79, 151.12, 149.03, 147.40, 141.58, 136.25, 131.66, 128.99, 127.82, 127.18, 125.78, 123.17, 115.96, 110.99, 106.49, 52.72, 48.91, 46.35.

### 4-(4-(4-(2,4-difluorophenyl)piperazin-1-yl)quinazolin-6-yl)pyridin-2-amine, 39

Compound 39 was prepared via General Scheme 1 via the two step procedure reported for the preparation of compound 1. The two steps are reported below.

### 6-bromo-4-(4-(2,4-difluorophenyl)piperazin-1-yl)quinazoline, 116

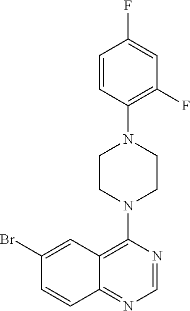 [SMILES: Fc1ccc(N2CCN(c3ncnc4ccc(Br)cc34)CC2)c(F)c1]

In a 5 mL Biotage microwave vial, sodium bicarbonate (17 mg, 1 eq, 0.20 mmol) and 1-(2,4-difluorophenyl)piperazine (40 mg, 1 eq, 0.20 mmol) were added with EtOH (0.5 mL, 0.4 M) and stirred for 10 mins. 6-bromo-4-chloroquinazoline (49 mg, 1 eq, 0.20 mmol) was added, vial sealed, and stirred at 25° C. for 12 h. Upon completion, the reaction was extracted in EtOAc, washed with brine, dried over sodium sulfate, and concentrated. The crude product was dry-loaded onto silica gel and purified on a 12 g silica gel column (0-10% EtOAc/Hexanes) to give 6-bromo-4-(4-(2,4-difluorophenyl)piperazin-1-yl)quinazoline, 116 (48 mg, 120 mol, 59%). LCMS [M+H]⁺ 406. 1H NMR (500 MHz, CDCl3) δ 8.73 (s, 1H), 8.07 (d, J=2.1 Hz, 2H), 7.89 (dd, J=8.7, 2.1 Hz, 1H), 6.95 (td, J=9.0, 5.6 Hz, 1H), 6.85 (tdd, J=11.9, 8.5, 2.9 Hz, 2H), 4.12 (s, 4H), 3.25 (t, J=4.9 Hz, 4H).

### 4-(4-(4-(2,4-difluorophenyl)piperazin-1-yl)quinazolin-6-yl)pyridin-2-amine, 39

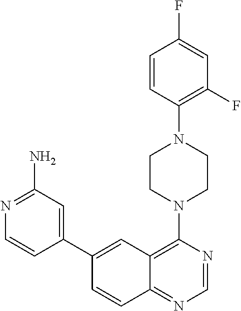 [SMILES: Nc1cc(-c2ccc3ncnc(N4CCN(c5ccc(F)cc5F)CC4)c3c2)ccn1]

In a 5 mL microwave vial, 6-chloro-4-(4-(2,4-difluorophenyl)piperazin-1-yl)quinazoline (49 mg, 1 eq, 0.14 mmol), 4-(4,4,5,5-tetramethyl-1,3,2-dioxaborolan-2-yl)pyridin-2-amine (36 mg, 1.2 eq, 0.16 mmol), tripotassium phosphate (120 mg, 4 eq, 0.54 mmol), and [1,1′-Bis(diphenylphosphino)ferrocene]dichloropalladium(II), complex with dichloromethane (8.9 mg, 0.08 eq, 11 μmol) were added. The mixture was capped, purged with argon, then injected with degassed 4:1 dioxane:H₂O (0.8 mL, 0.2 M), and was heated at 80° C. for 6 h. After this time, the mixture was cooled to room temperature, diluted with ethyl acetate (30 mL), and washed with water (10 mL). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (0-10% MeOH/DCM) to afford 4-(4-(4-(2,4-difluorophenyl)piperazin-1-yl)quinazolin-6-yl)pyridin-2-amine, 39, (27 mg, 65 μmol, 48%) as a light brown solid. LCMS [M+H]⁺ 419. 1H NMR (400 MHz, CDCl3) δ 8.78 (s, 1H), 8.14 (d, J=5.4 Hz, 1H), 8.09 (d, J=1.9 Hz, 1H), 8.03-7.76 (m, 2H), 7.01-6.89 (m, 2H), 6.89-6.77 (m, 3H), 4.91 (s, 2H), 4.01 (t, J=4.9 Hz, 4H), 3.24 (t, J=4.9 Hz, 4H).

### 4-(4-(4-(2-fluorophenyl)piperazin-1-yl)pyrido[3,2-d]pyrimidin-6-yl)isoxazole, 40

Compound 40 was prepared via General Scheme 1 via the two step procedure reported for the preparation of compound 1. The second step is reported below.

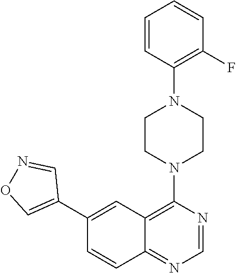 [SMILES: Fc1ccccc1N1CCN(c2ncnc3ccc(-c4cnoc4)cc23)CC1]

In a 5 mL microwave vial, 6-bromo-4-(4-(2-fluorophenyl)piperazin-1-yl)pyrido[3,2-d]pyrimidine (60 mg, 1 eq, 0.15 mmol), 4-(4,4,5,5-tetramethyl-1,3,2-dioxaborolan-2-yl)isoxazole (36 mg, 1.2 eq, 0.19 mmol), tripotassium phosphate (130 mg, 4 eq, 0.62 mmol), and [1,1′-Bis(diphenylphosphino)ferrocene]dichloropalladium(II) (9.0 mg, 0.08 eq, 12 mol) were added. The mixture was capped, purged with argon, then injected with degassed 4:1 Dioxane:H₂O (1.0 mL, 0.2 M), and was heated at 65° C. for 4 h. After this time, the mixture was cooled to room temperature, diluted with ethyl acetate (30 mL), and washed with water (10 mL). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (60-70% EtOAc/Hexanes) to afford 4-(4-(4-(2-fluorophenyl)piperazin-1-yl)pyrido[3,2-d]pyrimidin-6-yl)isoxazole, 40, (9.7 mg, 26 μmol, 17%) as a yellow solid. LCMS [M+H]⁺ 377. 1H NMR (400 MHz, DMSO) δ 9.78 (s, 1H), 9.30 (s, 1H), 8.55 (s, 1H), 8.23 (d, J=2.0 Hz, 2H), 7.23-7.09 (m, 3H), 7.01 (ddd, J=8.9, 5.7, 2.9 Hz, 1H), 4.59 (s, 4H), 3.27 (t, J=4.9 Hz, 4H). 13C NMR (101 MHz, DMSO-D6) δ 158.33, 158.28, 156.23, 154.45, 153.80, 148.53, 146.22, 145.25, 139.67, 139.59, 137.33, 132.78, 125.69, 124.90, 124.87, 122.67, 122.59, 121.55, 119.52, 119.50, 116.11, 115.91, 50.42.

### 4-(4-(4-(4-fluorobenzyl)-1,4-diazepan-1-yl)quinazolin-6-yl)pyridin-2-amine, 41

Compound 41 was prepared via General Scheme 1 via the two step procedure reported for the preparation of compound 1. The second step is reported below.

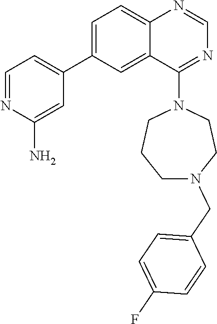 [SMILES: Nc1cc(-c2ccc3ncnc(N4CCCN(Cc5ccc(F)cc5)CC4)c3c2)ccn1]

Potassium acetate (156 mg, 3 Eq, 1.59 mmol), PdCl₂(dppf) (38 mg, 0.1 eq, 53 μmol), 4-(4,4,5,5-tetramethyl-1,3,2-dioxaborolan-2-yl)pyridin-2-amine (128 mg, 1.1 eq, 583 μmol), 6-bromo-4-(4-(4-fluorobenzyl)-1,4-diazepan-1-yl)quinazoline (220 mg, 1 eq, 530 μmol), were added to a MWV, the solids were degassed then dissolved in degassed solvent water (350 μL), 1,4-Dioxane (1.75 mL), the solution was heated to 60° C. and stirred using magnetic stirring for 16 h. After time elapsed the solution was diluted with EtOAc and filtered through a pad of celite. the filtrate was then concentrated via rotary evaporation and the residue was dissolved in EtOAc and washed twice with water and once with brine. The organic layer was collected and dried using Sodium Sulfate, which was then concentrated in vacuo, the residue was further purified using flash chromatography (MeOH/DCM 0-20%) resulting in a black sludge. Which was then further purified by dissolving in 1:1:1 DCM:MeOH:IPA and adding a metal scavenger Silica, and stirred for 72 h, the silica was then filtered off, and concentrated, resulting in 4-(4-(4-(4-fluorobenzyl)-1,4-diazepan-1-yl)quinazolin-6-yl)pyridin-2-amine, 41, (45 mg, 0.11 mmol, 20%) a white fluffy powder. LCMS [M+H]⁺ 429. ¹H NMR (400 MHz, DMSO) δ 8.46 (s, 1H), 8.21 (d, J=2.0 Hz, 1H), 7.97 (t, J=6.4 Hz, 2H), 7.78 (d, J=8.6 Hz, 1H), 7.29 (dd, J=8.3, 5.6 Hz, 2H), 7.08 (t, J=8.7 Hz, 2H), 6.83 (d, J=5.4 Hz, 1H), 6.74 (s, 1H), 3.98 (m, 4H), 3.56 (m, 2H), 2.86 (m, J=5.6 Hz, 2H), 2.61-2.53 (m, 2H), 2.01 (m, 2H).

### 4-(4-(4-fluorobenzyl)piperazin-1-yl)-6-(1-methyl-1H-pyrazol-4-yl)quinazoline, 42

Compound 42 was prepared via General Scheme 1 via the two step procedure reported for the preparation of compound 1. The second step is reported below.

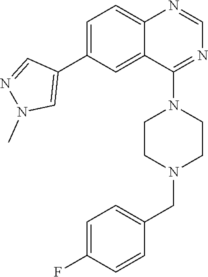 [SMILES: Cn1cc(-c2ccc3ncnc(N4CCN(Cc5ccc(F)cc5)CC4)c3c2)cn1]

Potassium phosphate, tribasic (198 mg, 3 eq, 935 μmol), PdCl₂(dppf) (22.8 mg, 0.1 eq, 31 mol), 1-methyl-4-(4,4,5,5-tetramethyl-1,3,2-dioxaborolan-2-yl)-1H-pyrazole (71 mg, 1.1 eq, 343 mol), 6-bromo-4-(4-(4-fluorobenzyl)piperazin-1-yl)quinazoline (125 mg, 1 eq, 312 mol), were added to a MWV, the solids were degassed then dissolved in degassed solvent water (0.30 mL), 1,4-Dioxane (1.5 mL), the solution was heated to 90° C. and stirred using magnetic stirring for 16 hours. After time elapsed the solution was diluted with EtOAc and filtered through a pad of celite. the filtrate was then concentrated via rotary evaporation and the residue was dissolved in EtOAc and washed twice with water and once with brine. The organic layer was collected and dried using Sodium Sulfate, which was then concentrated in vacuo, the residue was further purified using flash chromatography (MeOH/DCM 0-20%) resulting in a black sludge. Which was then further purified by dissolving in 1:1:1 DCM:MeOH:IPA and adding a metal scavenger silica, and stirred for 72 h, the silica was then filtered off resulting in 4-(4-(4-fluorobenzyl)piperazin-1-yl)-6-(1-methyl-1H-pyrazol-4-yl)quinazoline, 42, (101 mg, 251 μmol, 80%) a white fluffy powder. LCMS [M+H]⁺ 403. ¹H NMR (400 MHz, DMSO) δ 8.53 (s, 1H), 8.26 (s, 1H), 8.02-7.91 (m, 3H), 7.75 (d, J=8.7 Hz, 1H), 7.40-7.31 (m, 2H), 7.18-7.08 (m, 2H), 3.85 (s, 3H), 3.78-3.69 (m, 4H), 3.51 (s, 2H), 2.55 (t, J=4.8 Hz, 4H).

### 4-(4-(4-(4-fluorobenzyl)piperazin-1-yl)quinazolin-6-yl)pyridin-2-amine, 43

Compound 43 was prepared via General Scheme 1 via the two step procedure reported for the preparation of compound 1. The two steps are reported below.

### 6-bromo-4-(4-(4-fluorobenzyl)-1,4-diazepan-1-yl)quinazoline, 117

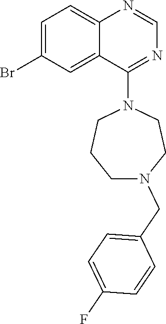 [SMILES: Fc1ccc(CN2CCCN(c3ncnc4ccc(Br)cc34)CC2)cc1]

To a microwave vial, 6-bromo-4-chloroquinazoline (0.300 g, 1 eq, 1.23 mmol), 1-(4-fluorobenzyl)-1,4-diazepane (513 mg, 478 L, 2 eq, 2.46 mmol), and a stir bar was added and the vial was capped. NMP (8.0 mL) followed by TEA (312 mg, 429 μL, 2.5 eq, 3.08 mmol) was added to the reaction vial, and the solution was heated to 80° C. and stirred for 2 hours. After the reaction was complete, the reaction mixture was diluted with EtOAc (50 mL), then washed with water (25 mL×4). The organic layer was separated, dried over sodium sulfate, filtered, and concentrated via rotary evaporation, the resulting residue was purified via column chromatography (MeOH:DCM 0-5%) resulting in 6-bromo-4-(4-(4-fluorobenzyl)-1,4-diazepan-1-yl)quinazoline (0.420 g, 1.01 mmol, 82%). LCMS [M+H]⁺ 416. This compound was used without further characterization.

### 4-(4-(4-(4-fluorobenzyl)piperazin-1-yl)quinazolin-6-yl)pyridin-2-amine, 43

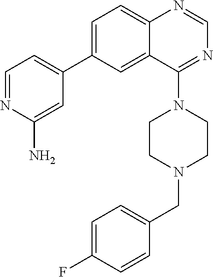 [SMILES: Nc1cc(-c2ccc3ncnc(N4CCN(Cc5ccc(F)cc5)CC4)c3c2)ccn1]

Potassium phosphate, tribasic (198 mg, 3 eq, 935 μmol), PdCl₂(dppf) (22.8 mg, 0.1 eq, 31.2 μmol), 4-(4,4,5,5-tetramethyl-1,3,2-dioxaborolan-2-yl)pyridin-2-amine (75 mg, 1.1 eq, 343 mol), 6-bromo-4-(4-(4-fluorobenzyl)piperazin-1-yl)quinazoline (125 mg, 1 eq, 312 mol), were added to a MWV, the solids were degassed then dissolved in degassed solvent water (0.30 mL), 1,4-Dioxane (1.5 mL), the solution was heated to 90° C. and stirred using magnetic stirring for 16 hours. After time elapsed the solution was diluted with EtOAc and filtered thru a pad of celite. the filtrate was then concentrated via rotary evaporation and the residue was dissolved in EtOAc and washed twice with water and once with brine. The organic layer was collected and dried using Sodium Sulfate, which was then concentrated in vacuo, the residue was further purified using flash chromatography (MeOH/DCM 0-20%) resulting in a black sludge. Which was then further purified by dissolving in 1:1:1 DCM:MeOH:IPA (3 mL) and adding a metal scavenger silica, and stirred for 72 h, the silica was then filtered off resulting in 4-(4-(4-(4-fluorobenzyl)piperazin-1-yl)quinazolin-6-yl)pyridin-2-amine, 43, (84 mg, 0.20 mmol, 65%) a white fluffy powder. LCMS [M+H]⁺ 415. ¹H NMR (400 MHz, DMSO) δ 8.59 (s, 1H), 8.10-7.94 (m, 3H), 7.84 (d, J=8.7 Hz, 1H), 7.33 (t, J=6.9 Hz, 2H), 7.12 (t, J=8.7 Hz, 2H), 6.84 (d, J=5.3 Hz, 1H), 6.75 (s, 1H), 3.76 (s, 4H), 3.49 (s, 2H), 2.56-2.48 (m, 4H).

### (R)-4-(4-(2-methyl-4-(pyridin-2-yl)piperazin-1-yl)quinazolin-6-yl)pyridin-2-amine, 44

Compound 44 was prepared via General Scheme 1 via the two step procedure reported for the preparation of compound 1. The two steps are reported below.

### (R)-6-bromo-4-(2-methyl-4-(pyridin-2-yl)piperazin-1-yl)quinazoline, 118

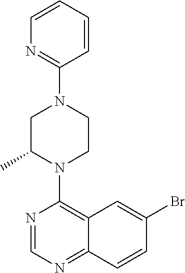 [SMILES: C[C@@H]1CN(c2ccccn2)CCN1c1ncnc2ccc(Br)cc12]

In a 5 mL microwave vial, 6-bromo-4-chloroquinazoline (96 mg, 1 eq, 0.39 mmol), sodium bicarbonate (100 mg, 3 eq, 1.2 mmol), and (R)-3-methyl-1-(pyridin-2-yl)piperazine (70 mg, 1.0 eq, 0.39 mmol) were added. The mixture was capped, DMSO (1.0 mL, 0.4 M) was injected, and stirred overnight at 60° C. for 18 hours. After this time, the mixture was cooled to room temperature, diluted with ethyl acetate (30 mL), and washed with water (10 mL×3). The aqueous layer was washed with ethyl acetate (20 mL×2). The combined organic layer was washed with brine (20 mL×3), dried over sodium sulfate, and evaporated in vacuo to obtain the crude product. The crude product was dry-loaded onto silica gel and purified on a 12 g silica gel column (0-50% EtOAc/Hexanes) to afford (R)-6-bromo-4-(2-methyl-4-(pyridin-2-yl)piperazin-1-yl)quinazoline, 118 (105 mg, 0.273 mmol, 69%). LCMS [M+H]⁺ 385. 1H NMR (500 MHz, CDCl3) δ 8.72 (s, 1H), 8.25-8.17 (m, 1H), 8.06 (d, J=2.2 Hz, 1H), 7.87 (dd, J=8.9, 2.1 Hz, 1H), 7.68-7.56 (m, 1H), 6.73 (s, 2H), 4.89 (s, 1H), 4.43-4.24 (m, 3H), 4.17 (d, J=12.9 Hz, 1H), 3.87 (t, J 12.2 Hz, 1H), 3.54 (d, J=12.9 Hz, 1H), 3.41-3.30 (m, 1H), 1.52 (d, J=6.7 Hz, 3H).

### (R)-4-(4-(2-methyl-4-(pyridin-2-yl)piperazin-1-yl)quinazolin-6-yl)pyridin-2-amine, 44

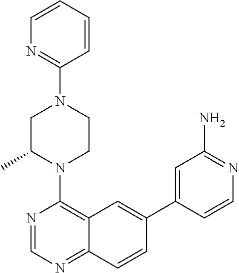 [SMILES: C[C@@H]1CN(c2ccccn2)CCN1c1ncnc2ccc(-c3ccnc(N)c3)cc12]

In a 5 mL microwave vial, (R)-6-bromo-4-(2-methyl-4-(pyridin-2-yl)piperazin-1-yl)quinazoline (105 mg, 1 eq, 0.273 mmol), 4-(4,4,5,5-tetramethyl-1,3,2-dioxaborolan-2-yl)pyridin-2-amine (72 mg, 1.2 eq, 0.328 mmol), tripotassium phosphate (232 mg, 4 eq, 1.09 mmol), and [1,1′-Bis(diphenylphosphino)ferrocene]dichloropalladium(II) (16 mg, 0.08 eq, 21.9 μmol) were added. The mixture was capped, purged with argon, then injected with degassed 4:1 Dioxane:H₂O (1.4 mL, 0.2 M), and was heated at 80° C. for 6 h. After this time, the mixture was cooled to room temperature, diluted with ethyl acetate (30 mL), and washed with water (10 mL×2). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (0-5% MeOH/DCM) to afford (R)-4-(4-(2-methyl-4-(pyridin-2-yl)piperazin-1-yl)quinazolin-6-yl)pyridin-2-amine, 44, (57 mg, 0.14 mmol, 52%) as a yellow solid. LCMS [M+H]⁺ 398. 1H NMR (500 MHz, CDCl3) δ 8.76 (s, 1H), 8.20 (dd, J=5.0, 1.8 Hz, 1H), 8.12 (s, 2H), 7.97 (q, J=8.7 Hz, 2H), 7.60-7.43 (m, 1H), 6.99-6.81 (m, 2H), 6.74-6.56 (m, 2H), 5.24 (s, 2H), 4.88-4.75 (m, 1H), 4.34-4.19 (m, 2H), 4.08 (dd, J=12.9, 3.0 Hz, 1H), 3.90-3.78 (m, 1H), 3.30 (td, J=12.1, 3.6 Hz, 1H), 2.61 (s, 1H), 1.48 (d, J=6.6 Hz, 3H).

### (S)-4-(4-(2-methyl-4-(pyridin-2-yl)piperazin-1-yl)quinazolin-6-yl)pyridin-2-amine, 45

Compound 45 was prepared via General Scheme 1 via the two step procedure reported for the preparation of compound 1. However this compound requires an additional SnAR step to add on a pyridyl group. This step is shown below.

### (S)-6-bromo-4-(2-methylpiperazin-1-yl)quinazoline, 119

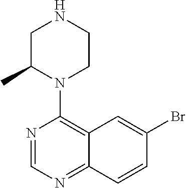 [SMILES: C[C@H]1CNCCN1c1ncnc2ccc(Br)cc12]

In a 5 mL Biotage microwave vial, sodium bicarbonate (311 mg, 3 eq, 3.70 mmol) and tert-butyl (S)-3-methylpiperazine-1-carboxylate (247 mg, 1 eq, 1.23 mmol) were added with DMSO (3.08 mL, 0.4 M) and stirred for 10 mins. 6-bromo-4-chloroquinazoline (300 mg, 1 eq, 1.23 mmol) was added, vial sealed, and stirred at 60° C. for 6 hours. Upon completion, the reaction was extracted in EtOAc, washed with brine, dried over sodium sulfate, concentrated, and dry loaded onto silicia and purified on a 12 g silica gel column from (0-15% EtOAc:Hexanes) to afford tert-butyl (S)-4-(6-bromoquinazolin-4-yl)-3-methylpiperazine-1-carboxylate (369 mg, 907.7 mol, 73.7%) as a yellow solid. LCMS [M+H]⁺ 407. 1H NMR (400 MHz, CDCl₃) δ 8.72 (s, 1H), 7.97 (d, J=2.1 Hz, 1H), 7.89 (d, J=8.9 Hz, 1H), 7.83 (dd, J=9.0, 2.0 Hz, 1H), 4.65 (s, 1H), 4.32-3.79 (m, 3H), 3.62 (t, J=13.4 Hz, 1H), 3.39-3.01 (m, 2H), 1.49 (s, 9H), 1.41 (d, J=6.9 Hz, 3H). In a scintillation vial, tert-butyl (S)-4-(6-bromoquinazolin-4-yl)-3-methylpiperazine-1-carboxylate (369 mg, 1.0 eq, 907 μmol) was dissolved in DCM:TFA (4:1, 9.5 mL) and stirred at room temperature overnight and basified with 1N NaOH. The mixture was extracted with DCM, washed with 10% NaOH, water, brine, and the organic layer was dried over sodium sulfate, and concentrated to afford (S)-6-bromo-4-(2-methylpiperazin-1-yl)quinazoline, 119, (213 mg, 693 μmol, 56%). LCMS [M+H]⁺ 307. 1H NMR (400 MHz, CDCl₃) δ 8.73 (s, 1H), 7.98 (d, J=2.0 Hz, 1H), 7.84-7.73 (m, 3H), 4.56 (td, J=7.0, 3.7 Hz, 1H), 3.99 (dt, J=13.7, 3.1 Hz, 1H), 3.63 (ddd, J=13.6, 11.4, 3.2 Hz, 1H), 3.18 (td, J=12.0, 3.5 Hz, 2H), 3.05 (td, J=11.9, 3.4 Hz, 1H), 2.95 (dt, J=12.4, 1.7 Hz, 1H), 1.49 (d, J=6.8 Hz, 3H).

### (S)-6-bromo-4-(2-methyl-4-(pyridin-2-yl)piperazin-1-yl)quinazoline, 120

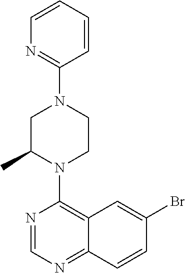 [SMILES: C[C@H]1CN(c2ccccn2)CCN1c1ncnc2ccc(Br)cc12]

To a 5 mL Biotage microwave vial was added potassium carbonate (297 mg, 3 eq, 2.08 mmol), (S)-6-bromo-4-(2-methylpiperazin-1-yl)quinazoline (213 mg, 1 eq, 0.693 mmol) in DMSO (1.39 mL, 0.5 M) and stirred at room temperature for 15 mins. Then 2-fluoropyridine (202 mg, 0.18 mL, 3 eq, 2.08 mmol) was injected via syringe, purged with nitrogen, and heated at 140° C. for 24 hours. The reaction mixture was then cooled to room temperature, extracted in EtOAc. The combined organic layers were washed with brine (×3) and water (×3), dried over sodium sulfate, filtered, concentrated, and dry-loaded onto silica gel and purified on a 12 g column (Hexanes/EtOAc 0-10%), affording (S)-6-bromo-4-(2-methyl-4-(pyridin-2-yl)piperazin-1-yl)quinazoline, 120, (81 mg, 0.21 mmol, 30%) as a yellow orange oil. LCMS [M+H]⁺ 385. 1H NMR (500 MHz, CDCl3) δ 8.72 (s, 1H), 8.22 (dt, J=7.5, 3.7 Hz, 1H), 8.06 (d, J=2.1 Hz, 1H), 7.99 (s, 1H), 7.88 (dd, J=8.8, 2.0 Hz, 1H), 7.62 (d, J=8.7 Hz, 1H), 6.74 (t, J=6.8 Hz, 2H), 4.90 (s, 1H), 4.45-4.24 (m, 2H), 4.18 (d, J=12.9 Hz, 1H), 3.88 (t, J=12.3 Hz, 1H), 3.54 (d, J=13.0 Hz, 1H), 3.38 (s, 1H), 1.52 (d, J=6.6 Hz, 3H).

### (S)-4-(4-(2-methyl-4-(pyridin-2-yl)piperazin-1-yl)quinazolin-6-yl)pyridin-2-amine, 45

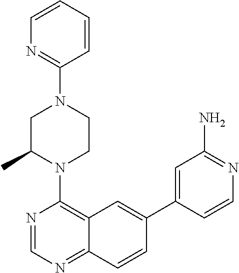 [SMILES: C[C@H]1CN(c2ccccn2)CCN1c1ncnc2ccc(-c3ccnc(N)c3)cc12]

In a 5 mL microwave vial, (S)-6-bromo-4-(2-methyl-4-(pyridin-2-yl)piperazin-1-yl)quinazoline (81 mg, 1 eq, 0.21 mmol), 4-(4,4,5,5-tetramethyl-1,3,2-dioxaborolan-2-yl)pyridin-2-amine (56 mg, 1.2 eq, 0.25 mmol), tripotassium phosphate (180 mg, 4 eq, 0.84 mmol), and [1,1′-bis(diphenylphosphino)ferrocene]dichloropalladium(II) (12 mg, 0.08 eq, 17 μmol) were added. The mixture was capped, purged with argon, then injected with degassed 4:1 Dioxane:H₂O (1.1 mL, 0.2 M), and was heated at 80° C. for 18 h. After this time, the mixture was cooled to room temperature, diluted with ethyl acetate (30 mL), and washed with water (10 mL×2). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (0-5% MeOH/DCM) to afford (S)-4-(4-(2-methyl-4-(pyridin-2-yl)piperazin-1-yl)quinazolin-6-yl)pyridin-2-amine, 45, (29 mg, 0.75 mmol, 35%) as a brown solid. LCMS [M+H]⁺ 398. 1H NMR (400 MHz, CDCl3) δ 8.76 (s, 1H), 8.24-8.17 (m, 1H), 8.16 (s, 1H), 8.09-7.85 (m, 3H), 7.53 (t, J=7.6 Hz, 1H), 7.09 (s, 1H), 6.96 (d, J=5.4 Hz, 1H), 6.77-6.62 (m, 2H), 4.87 (s, 1H), 4.29 (t, J=14.9 Hz, 2H), 4.10 (d, J=12.7 Hz, 1H), 3.88 (t, J=12.3 Hz, 1H), 3.52 (d, J=13.0 Hz, 1H), 3.33 (t, J=11.8 Hz, 1H), 1.50 (d, J=6.6 Hz, 3H). 13C NMR (126 MHz, DMSO) δ 163.42, 160.58, 159.10, 153.94, 151.63, 148.71, 147.53, 147.15, 137.58, 135.09, 130.96, 128.82, 122.67, 116.11, 112.86, 110.07, 106.73, 105.21, 52.29, 48.57, 44.27, 43.79, 15.73.

### (4-(6-(2-aminopyridin-4-yl)quinazolin-4-yl)piperazin-1-yl)(2-fluorophenyl)methanone, 46

Compound 46 was prepared via General Scheme 1 via the two step procedure reported for the preparation of compound 1. The two steps are reported below.

### (4-(6-bromoquinazolin-4-yl)piperazin-1-yl)(2-fluorophenyl)methanone, 121

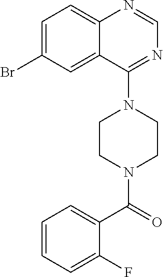 [SMILES: O=C(c1ccccc1F)N1CCN(c2ncnc3ccc(Br)cc23)CC1]

Prepared in a similar manner as compound 117. (4-(6-bromoquinazolin-4-yl)piperazin-1-yl)(2-fluorophenyl)methanone, 121, (287 mg, 0.691 mol, 84%). LCMS [M+H]⁺ 416. ¹H NMR (400 MHz, CDCl₃) δ 8.67 (s, 1H), 7.92 (s, 1H), 7.73 (s, 2H), 7.40-7.31 (m, 2H), 7.16 (t, J=7.5 Hz, 1H), 7.04 (t, J=9.1 Hz, 1H), 3.97-3.90 (m, 2H), 3.80 (t, J=5.1 Hz, 2H), 3.69 (t, J=5.0 Hz, 2H), 3.50 (d, J=6.3 Hz, 2H). ¹³C NMR (101 MHz, CDCl₃) δ 165.51, 163.55, 159.37, 156.91, 154.04, 150.24, 136.27, 131.81, 130.51, 129.38, 126.99, 124.99, 123.69, 119.23, 117.70, 116.03, 115.82, 49.92, 49.63, 46.65, 41.81.

### (4-(6-(2-aminopyridin-4-yl)quinazolin-4-yl)piperazin-1-yl)(2-fluorophenyl)methanone, 46

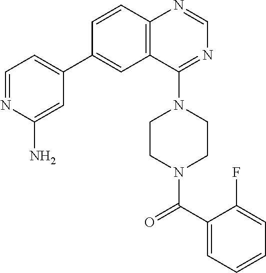 [SMILES: Nc1cc(-c2ccc3ncnc(N4CCN(C(=O)c5ccccc5F)CC4)c3c2)ccn1]

To a microwave vial potassium phosphate, tribasic (276 mg, 3 eq, 1.30 mmol), PdCl₂(dppf) (31 mg, 0.1 eq, 43 μmol), 4-(4,4,5,5-tetramethyl-1,3,2-dioxaborolan-2-yl)pyridin-2-amine (105 mg, 1.1 eq, 477 μmol), and (4-(6-bromoquinazolin-4-yl)piperazin-1-yl)(2-fluorophenyl)methanone (180 mg, 1 eq, 433 mol) was added and the vial was sealed. The solids were dissolved in 1,4-dioxane (2 mL) and water (0.4 mL), and the reaction mixture was heated to 90° C. where it stirred for 16 h. After time elapsed, the reaction mixture was cooled to room temperature, diluted with ethyl acetate and filtered through a thick celite pad. The filtrate was collected and concentrated via rotary evaporation. The resulting residue was dissolved in 50 mL of ethyl acetate and washed with water (3×50 mL). The organic layer was dried over sodium sulfate, filtered, and concentrated via rotary evaporation. The crude concentrate was purified via column chromatography (0-10% MeOH/DCM) producing (4-(6-(2-aminopyridin-4-yl)quinazolin-4-yl)piperazin-1-yl)(2-fluorophenyl)methanone, 46, (118 mg, 275 μmol, 63%). LCMS [M+H]⁺ 429. ¹H NMR (400 MHz, CDCl3) δ 8.74 (s, 1H), 8.08 (d, J=5.4 Hz, 1H), 8.02 (s, 1H), 7.98-7.87 (m, 2H), 7.39 (q, J=6.7 Hz, 2H), 7.20 (t, J=7.5 Hz, 1H), 7.08 (t, J=9.1 Hz, 1H), 6.89-6.79 (m, 2H), 3.99 (s, 2H), 3.90 (t, J=4.9 Hz, 2H), 3.78 (t, J=5.0 Hz, 2H), 3.54 (t, J=4.8 Hz, 2H).

### 4-(6,7-dimethoxy-3,4-dihydroisoquinolin-2(1H)-yl)-6-(1-methyl-1H-pyrazol-4-yl)quinazoline, 47

Compound 47 was prepared via General Scheme 1 via the two step procedure reported for the preparation of compound 1. The two steps are reported below.

### 6-bromo-4-(6,7-dimethoxy-3,4-dihydroisoquinolin-2(1H)-yl)quinazoline, 122

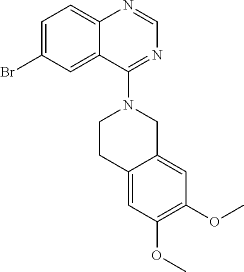 [SMILES: COc1cc2c(cc1OC)CN(c1ncnc3ccc(Br)cc13)CC2]

Prepared in a similar manner as compound 117. 6-bromo-4-(6,7-dimethoxy-3,4-dihydroisoquinolin-2(1H)-yl)quinazoline, 122, (486 mg, 1.21 mol, 85%). LCMS [M+H]⁺ 401. This compound was used without further characterization.

### 4-(6,7-dimethoxy-3,4-dihydroisoquinolin-2(1H)-yl)-6-(1-methyl-1H-pyrazol-4-yl)quinazoline, 47

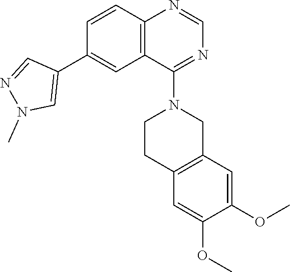 [SMILES: COc1cc2c(cc1OC)CN(c1ncnc3ccc(-c4cnn(C)c4)cc13)CC2]

To a microwave vial potassium phosphate, tribasic (318 mg, 3 eq, 1.50 mmol), PdCl₂(dppf) (36.6 mg, 0.1 eq, 50.0 μmol), 1-methyl-4-(4,4,5,5-tetramethyl-1,3,2-dioxaborolan-2-yl)-1H-pyrazole (114 mg, 1.1 eq, 550 mol), and 6-bromo-4-(6,7-dimethoxy-3,4-dihydroisoquinolin-2(1H)-yl)quinazoline (200 mg, 1 eq, 500 mol) was added and the vial was sealed. The solids were dissolved in 1,4-dioxane (2.0 mL) and water (0.40 mL), and the reaction mixture was heated to 90° C. where it stirred for 16 hours. After time elapsed, the reaction mixture was cooled to room temperature, diluted with ethyl acetate, and filtered through a thick celite pad. The filtrate was collected and concentrated via rotary evaporation. The resulting residue was dissolved in 50 mL of ethyl acetate and washed with water (3×50 mL). The organic layer was dried over sodium sulfate, filtered, and concentrated via rotary evaporation. The crude concentrate was purified via column chromatography (0-10% MeOH/DCM) producing 4-(6,7-dimethoxy-3,4-dihydroisoquinolin-2(1H)-yl)-6-(1-methyl-1H-pyrazol-4-yl)quinazoline, 47, (169 mg, 421 μmol, 84%). LCMS [M+H]⁺ 402. ¹H NMR (400 MHz, CDCl₃) δ 8.61 (s, 1H), 8.34 (s, 1H), 8.13 (d, J=1.8 Hz, 1H), 8.09-7.97 (m, 2H), 7.81 (d, J=8.6 Hz, 1H), 6.90 (s, 1H), 6.82 (s, 1H), 4.90 (s, 2H), 4.01 (t, J=5.7 Hz, 2H), 3.95 (s, 3H), 3.77 (d, J=7.4 Hz, 6H), 3.08 (t, J=5.7 Hz, 2H).

### 4-(4-(4-(2-fluorophenyl)piperidin-1-yl)quinazolin-6-yl)pyridin-2-amine, 48

Compound 48 was prepared via General Scheme 1 via the two step procedure reported for the preparation of compound 1. The second step is reported below.

### 6-bromo-4-(4-(2-fluorophenyl)piperidin-1-yl)quinazoline, 123

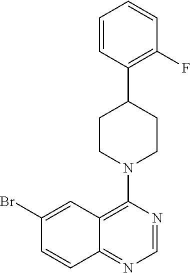 [SMILES: Fc1ccccc1C1CCN(c2ncnc3ccc(Br)cc23)CC1]

Prepared in similar manner as compound 126. 6-bromo-4-(4-(2-fluorophenyl)piperidin-1-yl)quinazoline, 123, (143 mg, 370 μmol, 66%). LCMS [M+H]⁺ 387. 1H NMR (500 MHz, CDCl3) δ 8.73 (s, 1H), 8.04 (d, J=1.9 Hz, 1H), 7.86-7.76 (m, 2H), 7.28 (dd, J=7.6, 1.8 Hz, 1H), 7.22 (tdd, J=7.4, 5.2, 1.8 Hz, 1H), 7.13 (td, J=7.5, 1.3 Hz, 1H), 7.05 (ddd, J=10.7, 8.2, 1.3 Hz, 1H), 4.53 (dt, J=13.4, 2.3 Hz, 2H), 3.37-3.28 (m, 2H), 3.25 (ddt, J=11.7, 8.2, 4.2 Hz, 1H), 2.08-1.94 (m, 4H).

### 4-(4-(4-(2-fluorophenyl)piperidin-1-yl)quinazolin-6-yl)pyridin-2-amine, 48

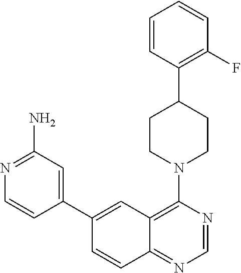 [SMILES: Nc1cc(-c2ccc3ncnc(N4CCC(c5ccccc5F)CC4)c3c2)ccn1]

In a 5 mL microwave vial, 6-bromo-4-(4-(2-fluorophenyl)piperidin-1-yl)quinazoline (143 mg, 1 eq, 0.370 mmol), 4-(4,4,5,5-tetramethyl-1,3,2-dioxaborolan-2-yl)pyridin-2-amine (97 mg, 1.2 eq, 0.444 mmol), tripotassium phosphate (314 mg, 4 eq, 1.48 mmol), and [1,1′-Bis(diphenylphosphino)ferrocene]dichloropalladium(II) (21 mg, 0.08 eq, 29 μmol) were added. The mixture was capped, purged with argon, then injected with degassed 4:1 Dioxane:H₂O (1.9 mL, 0.2 M), and was heated at 80° C. for 18 h. After this time, the mixture was cooled to room temperature, diluted with ethyl acetate (30 mL), and washed with water (10 mL×2). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (0-5% MeOH/DCM) to 4-(4-(4-(2-fluorophenyl)piperidin-1-yl)quinazolin-6-yl)pyridin-2-amine, 48. (39 mg, 98 μmol, 26%) as a pale yellow solid. LCMS [M+H]⁺ 400. 1H NMR (500 MHz, CDCl3) δ 8.76 (s, 1H), 8.10 (d, J=2.1 Hz, 2H), 7.98 (d, J=8.7 Hz, 1H), 7.94 (dd, J=8.7, 1.9 Hz, 1H), 7.29 (dd, J=7.5, 1.8 Hz, 1H), 7.22 (tdd, J=7.4, 5.2, 1.8 Hz, 1H), 7.13 (td, J=7.5, 1.3 Hz, 1H), 7.05 (ddd, J=10.6, 8.2, 1.3 Hz, 1H), 6.94 (dd, J=5.6, 1.5 Hz, 1H), 6.84 (s, 1H), 5.18 (s, 2H), 4.58 (d, J=13.2 Hz, 2H), 3.41-3.30 (m, 2H), 3.30-3.21 (m, 1H), 2.09-1.97 (m, 4H). 13C NMR (126 MHz, CDCl3) δ 164.98, 161.77, 159.82, 158.44, 154.81, 152.20, 150.49, 146.60, 135.16, 131.87, 131.75, 131.20, 129.50, 128.15, 128.08, 127.75, 127.71, 124.49, 124.46, 123.74, 116.76, 115.76, 115.58, 112.51, 107.12, 50.88, 35.97, 32.05.

### 4-(4-(4-fluorobenzyl)piperazin-1-yl)-6-(1H-pyrrolo[2,3-b]pyridin-3-yl)quinazoline, 49

Compound 49 was prepared via General Scheme 1 via the two step procedure reported for the preparation of compound 1. The two steps are reported below.

### 6-bromo-4-(4-(4-fluorobenzyl)piperazin-1-yl)quinazoline, 124

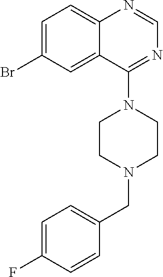 [SMILES: Fc1ccc(CN2CCN(c3ncnc4ccc(Br)cc34)CC2)cc1]

Prepared in a similar manner as compound 117. 6-bromo-4-(4-(4-fluorobenzyl)piperazin-1-yl)quinazoline, 124, (mg amount, mol, 99%). LCMS [M+H]⁺ 402. This compound was used without further characterization.

### 4-(4-(4-fluorobenzyl)piperazin-1-yl)-6-(1H-pyrrolo[2,3-b]pyridin-3-yl)quinazoline, 49

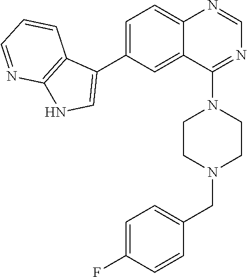 [SMILES: Fc1ccc(CN2CCN(c3ncnc4ccc(-c5c[nH]c6ncccc56)cc34)CC2)cc1]

Potassium phosphate, tribasic (198 mg, 3 eq, 935 μmol), PdCl₂(dppf) (22.8 mg, 0.1 eq, 31.2 mol), tert-butyl 3-(4,4,5,5-tetramethyl-1,3,2-dioxaborolan-2-yl)-1H-pyrrolo[2,3-b]pyridine-1-carboxylate (118 mg, 1.1 eq, 343 μmol), 6-bromo-4-(4-(4-fluorobenzyl)piperazin-1-yl)quinazoline (125 mg, 1 eq, 312 mol), were added to a MWV, the solids were degassed then dissolved in degassed solvent water (350 L), 1,4-Dioxane (1.75 mL), the solution was heated to 90° C. and stirred using magnetic stirring for 16 hour. After time elapsed the solution was diluted with EtOAc and filtered through a pad of celite. the filtrate was then concentrated via rotary evaporation and the residue was dissolved in EtOAc and washed twice with water and once with brine. The organic layer was collected and dried using sodium sulfate, which was then concentrated in vacuo, the residue was further purified using flash chromatography (MeOH/DCM 0-20%) resulting in a black sludge, which was then further purified by dissolving in 1:1:1 DCM:MeOH:IPA and adding a metal scavenger silica, and stirred for 72 h, the silica was then filtered off resulting in 4-(4-(4-fluorobenzyl)piperazin-1-yl)-6-(1H-pyrrolo[2,3-b]pyridin-3-yl)quinazoline, 49, (50 mg, 0.11 mmol, 37%) a white fluffy powder. LCMS [M+H]⁺ 439. ¹H NMR (400 MHz, DMSO) δ 12.06 (s, 1H), 8.56 (s, 1H), 8.26 (dd, J=17.6, 6.3 Hz, 2H), 8.15 (d, J=8.7 Hz, 1H), 8.08 (s, 1H), 8.02 (d, J=2.6 Hz, 1H), 7.82 (d, J=8.7 Hz, 1H), 7.56-7.41 (m, 1H), 7.37-7.29 (m, 2H), 7.13 (dt, J=17.3, 8.2 Hz, 2H), 3.78-3.71 (m, 4H), 3.50 (s, 2H), 2.56 (t, J=4.7 Hz, 4H).

### 4-(4-(4-(thiazol-2-yl)piperazin-1-yl)quinazolin-6-yl)pyridin-2-amine, 50

Compound 50 was prepared via General Scheme 1 via the two step procedure reported for the preparation of compound 1. The two steps are reported below.

### 2-(4-(6-bromoquinazolin-4-yl)piperazin-1-yl)thiazole, 125

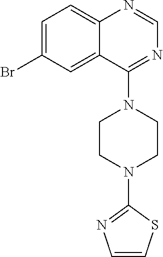 [SMILES: Brc1ccc2ncnc(N3CCN(c4nccs4)CC3)c2c1]

Prepared in a similar manner as compound 117. 2-(4-(6-bromoquinazolin-4-yl)piperazin-1-yl)thiazole, 125, (112 mg, 0.298 mol, 32%) LCMS [M+H]⁺ 377. This compound was used without further characterization.

### 4-(4-(4-(thiazol-2-yl)piperazin-1-yl)quinazolin-6-yl)pyridin-2-amine, 50

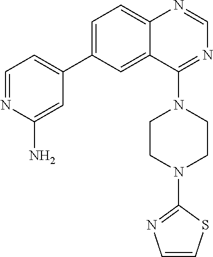 [SMILES: Nc1cc(-c2ccc3ncnc(N4CCN(c5nccs5)CC4)c3c2)ccn1]

To a microwave vial potassium phosphate, tribasic (190 mg, 3 eq, 893 mol), PdCl₂(dppf) (21.8 mg, 0.1 eq, 29.8 μmol), 4-(4,4,5,5-tetramethyl-1,3,2-dioxaborolan-2-yl)pyridin-2-amine (72 mg, 1.1 eq, 327 mol), and 2-(4-(6-bromoquinazolin-4-yl)piperazin-1-yl)thiazole (112 mg, 1 eq, 298 mol) was added and the vial was sealed. The solids were dissolved in 1,4-Dioxane (1 mL) and water (0.2 mL), and the reaction mixture was heated to 90° C. where it stirred for 16 hour. After time elapsed, the reaction mixture was cooled to room temperature, diluted with ethyl acetate and filtered through a thick celite pad. The filtrate was collected and concentrated via rotary evaporation. The resulting residue was dissolved in 50 mL of ethyl acetate and washed with water (3×50 mL). The organic layer was dried over sodium sulfate, filtered, and concentrated via rotary evaporation. The crude concentrate was purified via column chromatography (0-10% MeOH/DCM) producing 4-(4-(4-(thiazol-2-yl)piperazin-1-yl)quinazolin-6-yl)pyridin-2-amine, 50, (74 mg, 0.19 mmol, 64%). LCMS [M+H]⁺ 390. ¹H NMR (400 MHz, DMSO) δ 8.69 (s, 1H), 8.19 (d, J=2.0 Hz, 1H), 8.13-7.99 (m, 2H), 7.93 (d, J=8.7 Hz, 1H), 7.22 (d, J=3.6 Hz, 1H), 6.95 (dd, J=5.4, 1.6 Hz, 1H), 6.87 (dd, J=17.4, 2.6 Hz, 2H), 3.95 (dd, J=6.7, 3.8 Hz, 4H), 3.72-3.64 (m, 4H).

### 4-(4-(4-phenylpiperidin-1-yl)quinazolin-6-yl)pyridin-2-amine, 51

Compound 51 was prepared via General Scheme 1 via the two step procedure reported for the preparation of compound 1. The two steps are reported below.

### 6-bromo-4-(4-phenylpiperidin-1-yl)quinazoline, 126

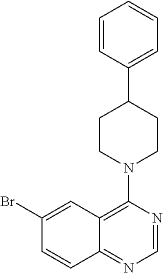 [SMILES: Brc1ccc2ncnc(N3CCC(c4ccccc4)CC3)c2c1]

A solution of 6-bromo-4-chloroquinazoline (300 mg, 1.2 eq, 1.23 mmol), 4-phenylpiperidine (166 mg, 1.0 eq, 1.03 mmol), and potassium carbonate (426 mg, 3.0 eq, 3.08 mmol) in DMSO (2.46 mL, 0.4 M) was stirred at 60° C. for 6 h. Upon cooling to room temperature, the reaction was extracted with EtOAc, washed with brine (×3) and water (×3), dried over anhydrous sodium sulfate, filtered, concentrated, and dry-loaded onto silica gel and purified on a 12 g silica gel column (0-10% EtOAc/Hexanes) to afford 6-bromo-4-(4-phenylpiperidin-1-yl)quinazoline, 126, (347 mg, 0.942 mmol, 92%). LCMS [M+H]⁺ 367. 1H NMR (400 MHz, CDCl3) δ 8.63 (d, J=2.6 Hz, 1H), 8.28 (t, J=5.8 Hz, 1H), 8.06 (t, J=2.4 Hz, 1H), 7.92 (dt, J=9.0, 2.4 Hz, 1H), 7.33 (qd, J=8.5, 2.3 Hz, 3H), 7.24 (d, J=4.2 Hz, 2H), 4.89 (s, 2H), 3.51 (t, J 13.0 Hz, 2H), 3.13-2.92 (m, 1H), 2.19 (s, 2H), 1.94 (tdd, J=15.7, 9.8, 3.5 Hz, 2H).

### 4-(4-(4-phenylpiperidin-1-yl)quinazolin-6-yl)pyridin-2-amine, 51

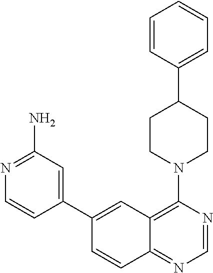 [SMILES: Nc1cc(-c2ccc3ncnc(N4CCC(c5ccccc5)CC4)c3c2)ccn1]

In a 5 mL microwave vial, 6-chloro-4-(4-phenylpiperidin-1-yl)quinazoline (57 mg, 1 eq, 0.18 mmol), 4-(4,4,5,5-tetramethyl-1,3,2-dioxaborolan-2-yl)pyridin-2-amine (46 mg, 1.2 eq, 0.21 mmol), tripotassium phosphate (150 mg, 4 eq, 0.70 mmol), and [1,1′-Bis(diphenylphosphino)ferrocene]dichloropalladium(II) (10 mg, 0.08 eq, 14 μmol) were added. The mixture was capped, purged with argon, then injected with degassed 4:1 dioxane:H₂O (0.9 mL, 0.2 M), and was heated at 80° C. for 18 h. After this time, the mixture was cooled to room temperature, diluted with ethyl acetate (30 mL), and washed with water (10 mL×2). The aqueous layer was washed with ethyl acetate (20 mL×3). The combined organic layer was washed with brine (20 mL), dried over sodium sulfate, and evaporated in vacuo to obtain the crude product. The crude product was dry loaded onto silica gel and purified on a 12 g silica gel column (0-5% MeOH/DCM) to afford 4-(4-(4-phenylpiperidin-1-yl)quinazolin-6-yl)pyridin-2-amine, 51, (23 mg, 0.18 mmol, 34%) as a pale yellow solid. LCMS [M+H]⁺ 382. 1H NMR (400 MHz, CDCl3) δ 8.74 (s, 1H), 8.12 (d, J=5.5 Hz, 1H), 8.08 (s, 1H), 7.93 (q, J=8.6 Hz, 2H), 7.33 (t, J=7.5 Hz, 2H), 7.27 (s, 1H), 7.21 (d, J=7.3 Hz, 2H), 6.91 (d, J=5.3 Hz, 1H), 6.77 (s, 1H), 4.87 (s, 2H), 4.54 (d, J=13.1 Hz, 2H), 3.36-3.21 (m, 2H), 2.94-2.81 (m, 1H), 2.10-1.89 (m, 4H). 13C NMR (101 MHz, CDCl3) δ 165.06, 159.02, 154.76, 152.16, 149.70, 148.48, 145.28, 135.58, 131.24, 129.45, 128.76, 126.93, 126.73, 123.53, 116.85, 112.63, 106.51, 50.89, 42.98, 33.43.

### Step 1, General Scheme 4

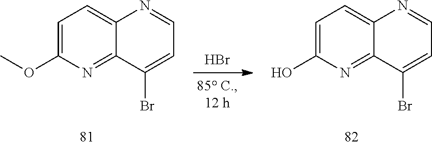

### 8-bromo-1,5-naphthyridin-2-ol, 82

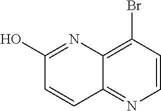 [SMILES: Oc1ccc2nccc(Br)c2n1]

In a 5 mL microwave vial, 8-bromo-2-methoxy-1,5-naphthyridine (54 mg, 1 eq, 0.23 mmol) was added followed by dropwise addition of hydrogen bromide (0.37 g, 0.25 mL, 20 eq, 4.5 mmol) at room temperature. The resulting mixture was heated at 85° C. for 12 hours. Upon completion, excess hydrogen bromide was evaporated in vacuo. The resulting crude mixture was dissolved in DCM:MeOH (9:1) and filtered off to afford 8-bromo-1,5-naphthyridin-2-ol, 82, (37 mg, 0.16 mmol, 73%) as a tan solid. LCMS [M+H]⁺ 226. 1H NMR (500 MHz, DMSO) δ 8.34 (d, J=5.0 Hz, 1H), 7.97 (d, J=9.7 Hz, 1H), 7.90 (d, J=5.0 Hz, 1H), 6.86 (d, J=9.7 Hz, 1H), 4.41 (s, 1H).

### Step 2, General Scheme 4

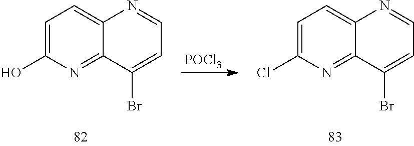

### 8-bromo-2-chloro-1,5-naphthyridine, 83

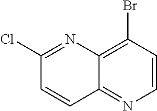 [SMILES: Clc1ccc2nccc(Br)c2n1]

In a 5 mL microwave vial, a mixture of 8-bromo-1,5-naphthyridin-2-ol (165 mg, 1 eq, 733 μmol) in phosphoryl trichloride (674 mg, 411 μL, 6 eq, 4.40 mmol) was refluxed for 2 h. Upon completion, the reaction mixture was added dropwise to ice water and extracted in ethyl acetate. The combined organic layer was washed with brine (20×2 mL), dried over sodium sulfate, filtered, and concentrated in vacuo to obtain the crude product. The crude product was dry-loaded onto silica gel and purified on a 12 g silica gel column (0-50% EtOAc/Hexanes) to afford 8-bromo-2-chloro-1,5-naphthyridine, 83, (94 mg, 386 μmol, 40%). LCMS [M+H]+ 244. 1H NMR (500 MHz, CDCl3) δ 8.85 (d, J=4.8 Hz, 1H), 8.39 (d, J=8.8 Hz, 1H), 7.78 (d, J=4.7 Hz, 1H), 7.70 (d, J=8.7 Hz, 1H).

### Step 3, General Scheme 4

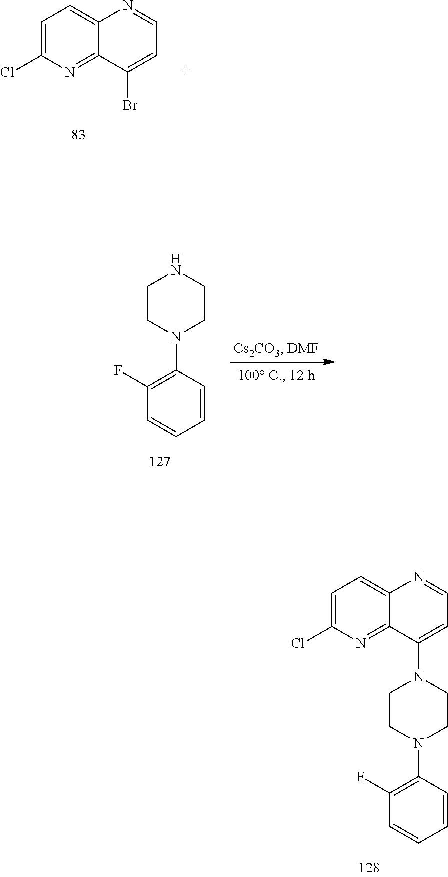 [SMILES: Clc1ccc2nccc(Br)c2n1 ; Fc1ccccc1N1CCNCC1 ; Fc1ccccc1N1CCN(c2ccnc3ccc(Cl)nc23)CC1]

### 2-chloro-8-(4-(2-fluorophenyl)piperazin-1-yl)-1,5-naphthyridine, 128

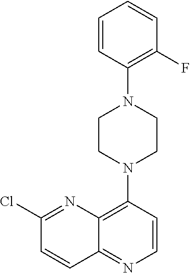 [SMILES: Fc1ccccc1N1CCN(c2ccnc3ccc(Cl)nc23)CC1]

In a 5 mL Biotage microwave vial, 1-(2-fluorophenyl)piperazine (18 mg, 16 μL, 1.2 eq, 99 mol) and cesium carbonate (54 mg, 2 eq, 0.16 mmol) were added, injected with DMF (0.8 mL, 0.1M), and stirred for 10 mins. 8-bromo-2-chloro-1,5-naphthyridine (20 mg, 1 eq, 82 μmol) was added, vial capped, and heated at 100° C. for 3 h. After this time, the mixture was cooled to room temperature, diluted with ethyl acetate (30 mL), and washed with water (10 mL). The aqueous layer was washed with ethyl acetate (20 mL×2). The combined organic layer was washed with brine (20 mL×2), dried over sodium sulfate, and evaporated in vacuo to obtain the crude product. The crude product was dry-loaded onto silica gel and purified on a 12 g silica gel column (0-5% MeOH/DCM) to afford 2-chloro-8-(4-(2-fluorophenyl)piperazin-1-yl)-1,5-naphthyridine, 128, (24 mg, 69 μmol, 84%). LCMS [M+H]⁺ 343. 1H NMR (500 MHz, CDCl3) δ 8.46 (d, J=5.0 Hz, 1H), 7.67 (s, 1H), 7.33 (t, J=9.5 Hz, 1H), 7.19-7.03 (m, 3H), 7.03-6.95 (m, 2H), 4.05 (s, 4H), 3.29-3.22 (m, 4H).

### Step 4, General Scheme 4

### 4-(8-(4-(2-fluorophenyl)piperazin-1-yl)-1,5-naphthyridin-2-yl)pyridin-2-amine, 52

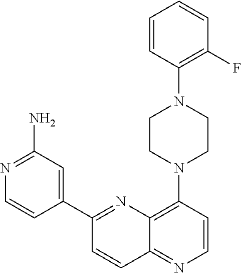 [SMILES: Nc1cc(-c2ccc3nccc(N4CCN(c5ccccc5F)CC4)c3n2)ccn1]

In a 5 mL Biotage microwave vial, 2-chloro-8-(4-(2-fluorophenyl)piperazin-1-yl)-1,5-naphthyridine (23 mg, 1 eq, 67 μmol), 4-(4,4,5,5-tetramethyl-1,3,2-dioxaborolan-2-yl)pyridin-2-amine (16 mg, 1.2 eq, 74 μmol), [1,1′-Bis(diphenylphosphino)ferrocene]dichloropalladium(II), complex with dichloromethane (5.5 mg, 0.08 eq, 6.7 μmol), and cesium carbonate (66 mg, 3 eq, 200 μmol), were added. The vial was capped, purged with nitrogen, then injected with degassed 4:1 Dioxane:H₂O (1.3 mL, 52 mM), and was heated at 95° C. for 12 hrs. After this time, the mixture was cooled to room temperature, diluted with ethyl acetate (30 mL), and washed with water (20 mL). The aqueous layer was washed with ethyl acetate (20 mL×2). The combined organic layer was washed with brine (20 mL×2), dried over sodium sulfate, and evaporated in vacuo to obtain the crude product. The crude product was dry-loaded onto silica gel and purified on a 12 g silica gel column (50-100% EtOAc/Hexanes) to afford 4-(8-(4-(2-fluorophenyl)piperazin-1-yl)-1,5-naphthyridin-2-yl)pyridin-2-amine, 52, (11 mg, 94 μmol, 28%) as a yellow solid. LCMS [M+H]⁺ 401. 1H NMR (500 MHz, CDCl3) δ 8.71 (d, J=4.4 Hz, 1H), 8.18 (dd, J=9.4, 4.6 Hz, 1H), 7.92 (d, J=6.3 Hz, 1H), 7.46 (d, J=4.4 Hz, 1H), 7.30 (d, J=9.4 Hz, 1H), 7.23 (dd, J=6.2, 1.4 Hz, 1H), 7.17 (s, 1H), 7.07 (dd, J=12.2, 6.7 Hz, 2H), 6.98 (qd, J=7.5, 4.9 Hz, 2H), 6.54 (s, 1H), 3.89-3.81 (m, 4H), 3.20 (t, J=5.2 Hz, 4H).

### 4-(8-(4-phenylpiperazin-1-yl)-1,5-naphthyridin-2-yl)pyridin-2-amine, 53

### 2-chloro-8-(4-phenylpiperazin-1-yl)-1,5-naphthyridine, 129

Compound 53 was prepared via General Scheme 4 via the four step procedure reported for the preparation of compound 52. The third and fourth steps are reported below.

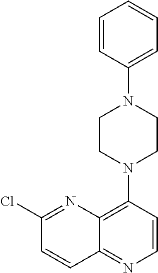 [SMILES: Clc1ccc2nccc(N3CCN(c4ccccc4)CC3)c2n1]

Prepared in a similar manner to Step 3, General Scheme 4 as used for compound 52. 2-chloro-8-(4-phenylpiperazin-1-yl)-1,5-naphthyridine, 129, (32 mg, 99 mol, 80%). LCMS [M+H]⁺ 325. 1H NMR (500 MHz, CDCl3) δ 8.47 (d, J=4.8 Hz, 1H), 8.17 (d, J=9.4 Hz, 1H), 7.59 (d, J=4.8 Hz, 1H), 7.41-7.28 (m, 3H), 7.01 (d, J=8.2 Hz, 2H), 6.93 (t, J=7.3 Hz, 1H), 4.03 (t, J=5.2 Hz, 4H), 3.37 (dd, J=6.1, 4.2 Hz, 4H).

### 4-(8-(4-phenylpiperazin-1-yl)-1,5-naphthyridin-2-yl)pyridin-2-amine, 53

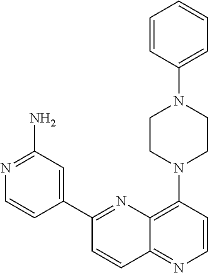 [SMILES: Nc1cc(-c2ccc3nccc(N4CCN(c5ccccc5)CC4)c3n2)ccn1]

Compound 53 was prepared via General Scheme 4 in an analogous manner to compound 52, step 4. 4-(8-(4-phenylpiperazin-1-yl)-1,5-naphthyridin-2-yl)pyridin-2-amine, 53, (11 mg, 98 μmol, 31%) as a yellow solid. LCMS [M+H]⁺ 383. 1H NMR (500 MHz, CDCl3) δ 8.68 (d, J=4.4 Hz, 1H), 8.18 (d, J=9.3 Hz, 1H), 8.14 (d, J=5.5 Hz, 1H), 7.46 (d, J=4.4 Hz, 1H), 7.33-7.27 (m, 3H), 7.13 (dd, J=5.4, 1.4 Hz, 1H), 7.01-6.94 (m, 3H), 6.89 (q, J=8.4 Hz, 1H), 4.86 (s, 2H), 3.86 (dd, J=6.5, 3.8 Hz, 4H), 3.30 (t, J=5.2 Hz, 4H). 13C NMR (101 MHz, CDCl3) δ 157.88, 156.56, 151.05, 148.26, 146.07, 145.68, 142.00, 140.82, 140.48, 139.04, 129.26, 123.57, 120.29, 116.40, 115.98, 112.77, 110.31, 77.32, 77.00, 76.68, 49.21, 45.05.

### 4-(4-((1R,5S)-3-(pyridin-2-yl)-3,8-diazabicyclo[3.2.1]octan-8-yl)quinazolin-6-yl)pyridin-2-amine, 54

Compound 54 was prepared via General Scheme 1 via the two step procedure reported for the preparation of compound 1. The two steps are reported below.

### 6-bromo-4-((1R,5S)-3-(pyridin-2-yl)-3,8-diazabicyclo[3.2.1]octan-8-yl)quinazoline, 130

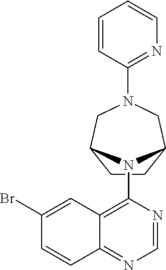 [SMILES: Brc1ccc2ncnc(N3[C@@H]4CC[C@H]3CN(c3ccccn3)C4)c2c1]

In a 5 mL Biotage microwave vial, potassium carbonate (178 mg, 3 eq, 1.29 mmol) and (1R,5S)-3-(pyridin-2-yl)-3,8-diazabicyclo[3.2.1]octane, trifluoroacetic acid (130 mg, 1 eq, 429 μmol) were added with DMF (2.14 mL, 0.2 M) and stirred for 10 mins. 6-bromo-4-chloroquinazoline (104 mg, 1 eq, 429 mol) was added, vial sealed, and stirred at 110° C. for 18 h. After this time, the mixture was cooled to room temperature, diluted with ethyl acetate (30 mL), and washed with water (10 mL). The aqueous layer was washed with ethyl acetate (20 mL). The combined organic layer was washed with brine (20 mL), dried over sodium sulfate, and evaporated in vacuo to obtain the crude product. The crude product was dry loaded onto silica gel and purified on a 12 g silica gel column (0-10% EtOAc/Hexanes) to afford 6-bromo-4-((1R,5S)-3-(pyridin-2-yl)-3,8-diazabicyclo[3.2.1]octan-8-yl)quinazoline, 130, (20 mg, 51 μmol, 12%). LCMS [M+H]⁺ 397. 1H NMR (400 MHz, CDCl₃) δ 8.68 (s, 1H), 8.20 (dd, J=5.2, 1.9 Hz, 1H), 8.13 (d, J=2.1 Hz, 1H), 7.84-7.72 (m, 2H), 7.50 (ddd, J=9.0, 7.1, 2.0 Hz, 1H), 6.71-6.60 (m, 2H), 4.96 (t, J=3.4 Hz, 2H), 4.10 (dd, J=12.0, 2.4 Hz, 2H), 3.39 (dd, J=11.9, 2.3 Hz, 2H), 2.05-1.85 (m, 4H).

### 4-(4-((1R,5S)-3-(pyridin-2-yl)-3,8-diazabicyclo[3.2.1]octan-8-yl)quinazolin-6-yl)pyridin-2-amine, 54

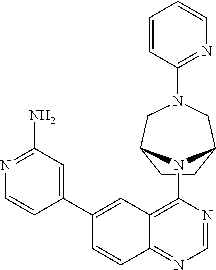 [SMILES: Nc1cc(-c2ccc3ncnc(N4[C@@H]5CC[C@H]4CN(c4ccccn4)C5)c3c2)ccn1]

A solution of (1R,5S)-3-(pyridin-2-yl)-3,8-diazabicyclo[3.2.1]octane (100 mg, 1.0 eq, 0.528 mmol) in NMP (2.64 mL, 0.18 M) was added triethylamine (134 mg, 184 L, 2.5 eq, 1.32 mmol) and tert-butyl (4-(4-chloroquinazolin-6-yl)pyridin-2-yl)carbamate (189 mg, 1 eq, 0.528 mmol). The mixture was stirred at 80° C. for 2 hrs. Upon cooling to room temperature, the reaction was extracted with EtOAc, washed with brine (×3) and water (×3), dried over anhydrous sodium sulfate, filtered, and concentrated in vacuo. A mixture of DCM:TFA (4:1, 2.2 mL) was added to the crude mixture and stirred at room temperature overnight and basified with 1N NaOH. The mixture was extracted with DCM, washed with 10% NaOH, water, brine, and the organic layer was dried over sodium sulfate, concentrated, and dry-loaded onto silica gel and purified on a 12 g column (DCM/MeOH 0-10%), affording 4-(4-((1R,5S)-3-(pyridin-2-yl)-3,8-diazabicyclo[3.2.1]octan-8-yl)quinazolin-6-yl)pyridin-2-amine, 54, (22 mg, 55 mol, 19%) as a yellow solid. LCMS [M+H]⁺ 410. 1H NMR (500 MHz, CDCl3) δ 8.71 (s, 1H), 8.24-8.16 (m, 2H), 8.11 (d, J=5.6 Hz, 1H), 7.95 (d, J=1.7 Hz, 2H), 7.51 (ddd, J=8.9, 7.1, 1.9 Hz, 1H), 6.92 (dd, J=5.5, 1.5 Hz, 1H), 6.80 (s, 1H), 6.70-6.60 (m, 2H), 5.08 (s, 2H), 5.01 (d, J=4.0 Hz, 2H), 4.12 (dd, J=12.0, 2.4 Hz, 2H), 3.43 (dd, J=11.9, 2.2 Hz, 2H), 2.04-1.86 (m, 4H). 13C NMR (126 MHz, CDCl3) δ 163.08, 159.89, 158.65, 155.08, 151.95, 150.14, 147.90, 147.24, 137.77, 135.73, 131.51, 129.48, 123.28, 117.03, 113.80, 112.50, 106.89, 57.79, 51.53, 41.17, 27.13.

### 4-(4-((1R,4R)-5-(pyridin-2-yl)-2,5-diazabicyclo[2.2.1]heptan-2-yl)quinazolin-6-yl)pyridin-2-amine, 55

Compound 55 was prepared via General Scheme 1 via the two step procedure reported for the preparation of compound 1. The two steps are reported below.

### 6-bromo-4-((1R,4R)-5-(pyridin-2-yl)-2,5-diazabicyclo[2.2.1]heptan-2-yl)quinazoline, 131

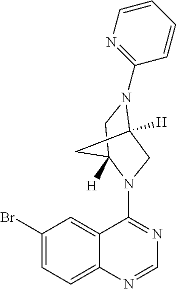 [SMILES: Brc1ccc2ncnc(N3C[C@H]4C[C@@H]3CN4c3ccccn3)c2c1]

A solution of (1S,4S)-2-(pyridin-2-yl)-2,5-diazabicyclo[2.2.1]heptane (43.5 mg, 1.0 eq, 0.248 mmol) in NMP (1.24 mL, 0.18 M) was added triethylamine (62.8 mg, 86.5 L, 2.5 eq, 0.621 mmol) and 6-bromo-4-chloroquinazoline (60 mg, 1 eq, 0.248 mmol). The mixture was stirred at 80° C. for 2 hrs. Upon cooling to room temperature, the reaction was extracted with EtOAc, washed with brine (×3) and water (×3), dried over anhydrous sodium sulfate, filtered, and concentrated, and dry-loaded onto silica gel and purified on a 12 g column (Hexanes/EtOAc 50%), affording 6-bromo-4-((1R,4R)-5-(pyridin-2-yl)-2,5-diazabicyclo[2.2.1]heptan-2-yl)quinazoline, 131, (91 mg, 0.24 mmol, 96%). LCMS [M+H]⁺ 383. 1H NMR (500 MHz, CDCl3) δ 8.55 (s, 1H), 8.12-8.02 (m, 2H), 7.74-7.60 (m, 2H), 7.39 (ddd, J=8.8, 7.1, 1.9 Hz, 1H), 6.52 (dd, J=7.1, 5.0 Hz, 1H), 6.29 (d, J=8.4 Hz, 1H), 5.39 (t, J=1.8 Hz, 1H), 5.13 (s, 1H), 4.24 (dd, J=9.1, 1.9 Hz, 1H), 3.91 (d, J=9.1 Hz, 1H), 3.71 (dd, J=9.4, 2.1 Hz, 1H), 3.58 (d, J=9.4 Hz, 1H), 2.13 (s, 2H).

### 4-(4-((1R,4R)-5-(pyridin-2-yl)-2,5-diazabicyclo[2.2.1]heptan-2-yl)quinazolin-6-yl)pyridin-2-amine, 55

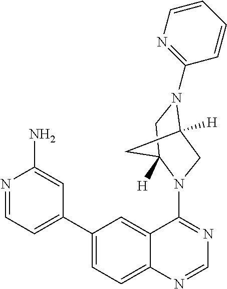 [SMILES: Nc1cc(-c2ccc3ncnc(N4C[C@H]5C[C@@H]4CN5c4ccccn4)c3c2)ccn1]

In a 5 mL microwave vial, 6-bromo-4-((1S,4S)-5-(pyridin-2-yl)-2,5-diazabicyclo[2.2.1]heptan-2-yl)quinazoline (65 mg, 1 eq, 0.17 mmol), 4-(4,4,5,5-tetramethyl-1,3,2-dioxaborolan-2-yl)pyridin-2-amine (45 mg, 1.2 eq, 0.2 mmol), tripotassium phosphate (140 mg, 4 eq, 0.68 mmol), and [1,1′-Bis(diphenylphosphino)ferrocene]dichloropalladium(II) (10 mg, 0.08 eq, 14 μmol) were added. The mixture was capped, purged with argon, then injected with degassed 4:1 Dioxane:H₂O (0.85 mL, 0.2 M), and was heated at 90° C. for 6 h. After this time, the mixture was cooled to room temperature, diluted with ethyl acetate (30 mL), and washed with water (10 mL×2). The aqueous layer was washed with ethyl acetate (20 mL). The combined organic layer was washed with brine (20 mL), dried over sodium sulfate, and evaporated in vacuo to obtain the crude product. The crude product was dry-loaded onto silica gel and purified on a 12 g silica gel column (0-5% MeOH/DCM) to 4-(4-((1R,4R)-5-(pyridin-2-yl)-2,5-diazabicyclo[2.2.1]heptan-2-yl)quinazolin-6-yl)pyridin-2-amine, 55, (39 mg, 98 μmol, 26%) as a white solid. LCMS [M+H]⁺ 396. 1H NMR (500 MHz, CDCl3) δ 8.61 (d, J=1.3 Hz, 1H), 8.22 (d, J=3.3 Hz, 1H), 8.07 (dd, J=5.4, 1.9 Hz, 1H), 8.02 (d, J=5.7 Hz, 1H), 7.88 (d, J=1.7 Hz, 2H), 7.48-7.37 (m, 1H), 6.96 (d, J=11.6 Hz, 1H), 6.87 (d, J=5.6 Hz, 1H), 6.55 (dt, J=7.6, 4.1 Hz, 1H), 6.35 (dd, J=8.6, 4.0 Hz, 1H), 5.67 (s, 2H), 5.50-5.40 (m, 1H), 5.16 (s, 1H), 4.43 (d, J=9.4 Hz, 1H), 4.07-3.98 (m, 1H), 3.77 (dt, J=9.5, 2.5 Hz, 1H), 3.65 (d, J=9.3 Hz, 1H), 2.27-2.12 (m, 2H). 13C NMR (126 MHz, CDCl3) δ 160.34, 157.77, 156.44, 154.98, 151.88, 151.08, 147.40, 143.53 138.09, 134.10, 130.87, 129.14, 123.70, 116.51, 112.71, 111.77, 108.01, 107.49, 60.64, 59.56, 57.16, 54.30, 36.73.

### 4-(4-(4-(pyridin-2-yl)-1,4-diazepan-1-yl)quinazolin-6-yl)pyridin-2-amine, 56

Compound 56 was prepared via General Scheme 1 via the two step procedure reported for the preparation of compound 1. The two steps are reported below.

### 6-bromo-4-(4-(pyridin-2-yl)-1,4-diazepan-1-yl)quinazoline, 132

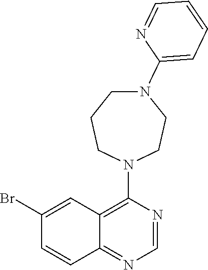 [SMILES: Brc1ccc2ncnc(N3CCCN(c4ccccn4)CC3)c2c1]

Prepared using same procedure as seen for compound 131. 6-bromo-4-(4-(pyridin-2-yl)-1,4-diazepan-1-yl)quinazoline, 132, (167 mg, 434 μmol). LCMS [M+H]⁺ 385. 1H NMR (500 MHz, CDCl3) δ 8.58 (s, 1H), 8.12 (t, J=1.3 Hz, 1H), 8.08 (dt, J=4.9, 1.7 Hz, 1H), 7.85-7.74 (m, 2H), 7.44 (ddd, J=8.9, 7.1, 2.0 Hz, 1H), 6.59-6.49 (m, 2H), 4.18 (t, J=5.3 Hz, 2H), 4.07 (t, J=5.4 Hz, 2H), 4.00-3.91 (m, 2H), 3.77 (t, J=6.0 Hz, 2H), 2.30-2.15 (m, 2H).

### 4-(4-(4-(pyridin-2-yl)-1,4-diazepan-1-yl)quinazolin-6-yl)pyridin-2-amine, 56

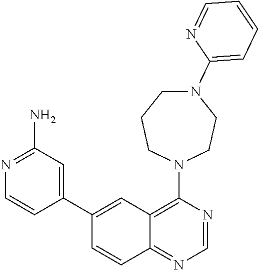 [SMILES: Nc1cc(-c2ccc3ncnc(N4CCCN(c5ccccn5)CC4)c3c2)ccn1]

Specifically compound 56 was prepared in analogous fashion to conditions employed for compound 31 (28 mg, 70 μmol, 18% yield). LCMS [M+H]⁺ 398. 1H NMR (500 MHz, CDCl3) δ 8.62 (s, 1H), 8.19 (s, 1H), 8.11 (s, 1H), 8.07 (d, J=5.0 Hz, 1H), 7.91 (s, 2H), 7.44 (t, J=8.0 Hz, 1H), 6.91 (d, J=5.1 Hz, 1H), 6.84 (s, 1H), 6.63-6.47 (m, 2H), 5.12 (s, 2H), 4.22 (d, J=5.5 Hz, 2H), 4.10 (d, J=5.3 Hz, 2H), 3.99 (d, J=5.7 Hz, 2H), 3.79 (t, J=6.2 Hz, 2H), 2.26 (s, 2H).

### 4-(4-(3-fluoropyridin-2-yl)piperazin-1-yl)-6-(1H-pyrrolo[2,3-b]pyridin-3-yl)quinazoline, 57

Compound 57 was prepared via General Scheme 1 via the two procedure reported for the preparation of compound 1. The two steps are reported below.

### 6-bromo-4-(4-(3-fluoropyridin-2-yl)piperazin-1-yl)quinazoline, 133

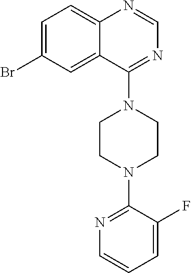 [SMILES: Fc1cccnc1N1CCN(c2ncnc3ccc(Br)cc23)CC1]

Prepared in a similar manner as compound 117. (mg amount, mol, 68%). LCMS [M+H]⁺ 389. ¹H NMR (400 MHz, CDCl₃) δ 8.67 (s, 1H), 7.99-7.90 (m, 2H), 7.76-7.65 (m, 2H), 7.24-7.12 (m, 1H), 6.72 (ddd, J=7.9, 4.8, 3.1 Hz, 1H), 3.94-3.80 (m, 4H), 3.68-3.58 (m, 4H). ¹³C NMR (101 MHz, CDCl₃) δ 163.45, 154.23, 151.25. 150.46, 149.49, 148.70, 142.80, 135.76, 130.46, 127.13, 123.37, 123.18, 118.60, 117.68, 116.33, 49.54, 47.36.

### 4-(4-(3-fluoropyridin-2-yl)piperazin-1-yl)-6-(1H-pyrrolo[2,3-b]pyridin-3-yl)quinazoline, 57

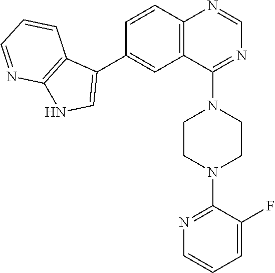 [SMILES: Fc1cccnc1N1CCN(c2ncnc3ccc(-c4c[nH]c5ncccc45)cc23)CC1]

Tert-butyl 3-(4-(4-(3-fluoropyridin-2-yl)piperazin-1-yl)quinazolin-6-yl)-1H-pyrrolo[2,3-b]pyridine-1-carboxylate (163 mg, 1 eq, 311 mol) was suspended in DCM (3 mL) and TFA (710 mg, 479 μL, 20 Eq, 6.229 mmol) added. The solution stirred for 2 h at room temperature, after time elapsed the reaction mixture was concentrated via rotary evaporation, the residue was dissolved in ethyl acetate and washed with 2M NaOH solution twice (2×75 mL). The organic layer was collected, dried with sodium sulfate, filtered and concentrated resulting in 4-(4-(3-fluoropyridin-2-yl)piperazin-1-yl)-6-(1H-pyrrolo[2,3-b]pyridin-3-yl)quinazoline, 57, (120 mg, 282 μmol, 90%). LCMS [M+H]⁺ 426. ¹H NMR (500 MHz, MeOD) δ 8.53 (s, 1H), 8.27 (dd, J=7.9, 1.5 Hz, 1H), 8.24-8.15 (m, 2H), 8.09 (dd, J=8.7, 1.9 Hz, 1H), 7.92 (dt, J=4.9, 1.4 Hz, 1H), 7.83 (d, J=8.7 Hz, 1H), 7.71 (s, 1H), 7.30 (ddd, J=13.1, 7.9, 1.5 Hz, 1H), 7.18 (dd, J=8.0, 4.8 Hz, 1H), 6.79 (ddd, J=8.0, 4.9, 3.2 Hz, 1H), 4.00-3.95 (m, 4H), 3.67-3.61 (m, 4H).

### 1-(1-(6-(1H-pyrrolo[2,3-b]pyridin-3-yl)quinazolin-4-yl)piperidin-4-yl)pyridin-2(1H)-one, 58

Compound 58 was prepared via General Scheme 1 via the procedure reported for the preparation of compound 1; however, an additional step was required to remove a BOC protecting group. The second and final steps are reported below.

### 1-(1-(6-bromoquinazolin-4-yl)piperidin-4-yl)pyridin-2(1H)-one, 134

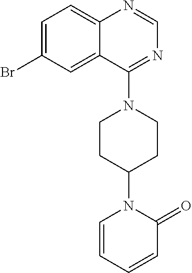 [SMILES: O=c1ccccn1C1CCN(c2ncnc3ccc(Br)cc23)CC1]

Prepared in a similar manner as compound 117. 1-(1-(6-bromoquinazolin-4-yl)piperidin-4-yl)pyridin-2(1H)-one, 134, (556 mg, 1.44 mol, 86%). LCMS [M+H]⁺ 386. ¹H NMR (500 MHz, CDCl₃) δ 8.41 (s, 1H), 7.84 (dd, J=5.1, 2.0 Hz, 1H), 7.69 (d, J=2.1 Hz, 1H), 7.48-7.38 (m, 2H), 7.26 (ddd, J=8.9, 7.1, 2.1 Hz, 1H), 6.54 (dd, J=7.1, 5.0 Hz, 1H), 6.43 (d, J=8.3 Hz, 1H), 5.10 (tt, J=7.6, 3.8 Hz, 1H), 3.75 (ddd, J=13.5, 7.3, 3.6 Hz, 2H), 3.42-3.34 (m, 2H), 1.92 (ddt, J 14.3, 7.4, 3.7 Hz, 2H), 1.71 (dtd, J=11.9, 7.8, 3.6 Hz, 2H).

### Tert-butyl 3-(4-(4-(2-oxopyridin-1(2H)-yl)piperidin-1-yl)quinazolin-6-yl)-1H-pyrrolo[2,3-b]pyridine-1-carboxylate, 135

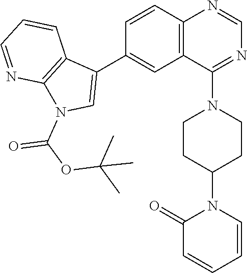 [SMILES: CC(C)(C)OC(=O)n1cc(-c2ccc3ncnc(N4CCC(n5ccccc5=O)CC4)c3c2)c2cccnc21]

To a microwave vial potassium phosphate, tribasic (306 mg, 3 eq, 1.44 mmol), PdCl₂(dppf) (35 mg, 0.1 eq, 48.0 μmol), tert-butyl 3-(4,4,5,5-tetramethyl-1,3,2-dioxaborolan-2-yl)-1H-pyrrolo[2,3-b]pyridine-1-carboxylate (174 mg, 1.05 eq, 504 μmol), and 1-(1-(6-bromoquinazolin-4-yl)piperidin-4-yl)pyridin-2(1H)-one (185 mg, 1 eq, 480 mol) was added and the vial was sealed. The solids were dissolved in 1,4-Dioxane (2.0 mL) and water (0.40 mL), and the reaction mixture was heated to 90° C. where it stirred for 16 hours. After time elapsed, the reaction mixture was cooled to room temperature, diluted with ethyl acetate, and filtered through a thick celite pad. The filtrate was collected and concentrated via rotary evaporation. The resulting residue was dissolved in 50 mL of ethyl acetate and washed with water (3×50 mL). The organic layer was dried over sodium sulfate, filtered, and concentrated via rotary evaporation. The crude concentrate was purified via column chromatography (0-10% MeOH/DCM) producing tert-butyl 3-(4-(4-(2-oxopyridin-1(2H)-yl)piperidin-1-yl)quinazolin-6-yl)-1H-pyrrolo[2,3-b]pyridine-1-carboxylate, 135, (126 mg, 242 mol, 50%). LCMS [M+H]⁺ 523. This compound was carried forward to the next step without other characterization.

### 1-(1-(6-(1H-pyrrolo[2,3-b]pyridin-3-yl)quinazolin-4-yl)piperidin-4-yl)pyridin-2(1H)-one, 58

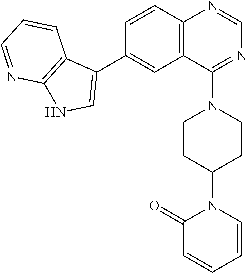 [SMILES: O=c1ccccn1C1CCN(c2ncnc3ccc(-c4c[nH]c5ncccc45)cc23)CC1]

Tert-butyl 3-(4-(4-(2-oxopyridin-1(2H)-yl)piperidin-1-yl)quinazolin-6-yl)-1H-pyrrolo[2,3-b]pyridine-1-carboxylate (126 mg, 1 eq, 242 mol) was suspended in DCM (2 mL) and TFA (553 mg, 373 L, 20 Eq, 4.85 mmol) added. The solution stirred for 2 h at room temperature, after time elapsed the reaction mixture was concentrated via rotary evaporation, the residue was dissolved in ethyl acetate and washed with 2M NaOH solution twice (2×75 mL). The organic layer was collected, dried with sodium sulfate, filtered and concentrated resulting in 1-(1-(6-(1H-pyrrolo[2,3-b]pyridin-3-yl)quinazolin-4-yl)piperidin-4-yl)pyridin-2(1H)-one, 58, (100 mg, 237 mol, 97%). LCMS [M+H]⁺ 423. ¹H NMR (400 MHz, CDCl₃) δ 12.20 (s, 1H), 8.78 (s, 1H), 8.49-8.43 (m, 1H), 8.30 (dd, J=8.0, 1.3 Hz, 1H), 8.21-8.08 (m, 2H), 8.08-7.95 (m, 2H), 7.70 (s, 1H), 7.59 (ddd, J=8.4, 7.1, 2.0 Hz, 1H), 7.30-7.20 (m, 1H), 6.88 (ddd, J=7.1, 5.0, 1.0 Hz, 1H), 6.77 (dt, J=8.3, 0.9 Hz, 1H), 5.45 (tt, J=7.6, 3.8 Hz, 1H), 4.23-4.11 (m, 2H), 3.76 (ddd, J=13.2, 8.2, 3.4 Hz, 2H), 2.29 (ddt, J=13.8, 7.3, 3.4 Hz, 2H), 2.14-2.02 (m, 2H).

### 1-(1-(6-(1H-pyrazol-4-yl)quinazolin-4-yl)piperidin-4-yl)pyridin-2(1H)-one, 59

Compound 59 was prepared via General Scheme 1 via the two step procedure reported for the preparation of compound 1. The second step is reported below.

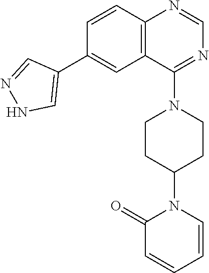 [SMILES: O=c1ccccn1C1CCN(c2ncnc3ccc(-c4cn[nH]c4)cc23)CC1]

To a microwave vial potassium phosphate, tribasic (306 mg, 3 eq, 1.44 mmol), PdCl₂(dppf) (35.1 mg, 0.1 eq, 48.0 μmol), tert-butyl 4-(4,4,5,5-tetramethyl-1,3,2-dioxaborolan-2-yl)-1H-pyrazole-1-carboxylate (148 mg, 1.05 eq, 504 mol), and 1-(1-(6-bromoquinazolin-4-yl)piperidin-4-yl)pyridin-2(1H)-one (185 mg, 1 eq, 480 μmol) was added and the vial was sealed. The solids were dissolved in 1,4-dioxane (2.0 mL) and water (0.40 mL), and the reaction mixture was heated to 90° C. where it stirred for 16 hours. After time elapsed, the reaction mixture was cooled to room temperature, diluted with ethyl acetate, and filtered through a thick celite pad. The filtrate was collected and concentrated via rotary evaporation. The resulting residue was dissolved in 50 mL of ethyl acetate and washed with water (3×50 mL). The organic layer was dried over sodium sulfate, filtered, and concentrated via rotary evaporation. The crude concentrate was purified via column chromatography (0-10% MeOH/DCM) producing 1-(1-(6-(1H-pyrazol-4-yl)quinazolin-4-yl)piperidin-4-yl)pyridin-2(1H)-one, 59, (123 mg, 330 μmol, 68%). LCMS [M+H]⁺ 373. ¹H NMR (400 MHz, CDCl₃) δ 8.74 (s, 1H), 8.19 (ddd, J=5.0, 2.0, 0.8 Hz, 1H), 8.00-7.83 (m, 3H), 7.67-7.37 (m, 2H), 6.91 (ddd, J=7.1, 5.1, 1.0 Hz, 1H), 6.80 (s, 1H), 6.78 (s, 1H), 5.46 (tt, J=7.7, 3.9 Hz, 1H), 4.14 (ddd, J=11.2, 6.8, 3.6 Hz, 2H), 3.82-3.67 (m, 2H), 2.31 (ddd, J=12.9, 7.2, 3.6 Hz, 2H), 2.08 (dtd, J=12.2, 8.0, 3.5 Hz, 2H).

### 6-(3-methyl-1H-pyrazol-4-yl)-4-(4-(pyridin-2-yl)piperazin-1-yl)quinazoline, 60

Compound 60 was prepared via General Scheme 1 via the two step procedure reported for the preparation of compound 1. The second step is reported below.

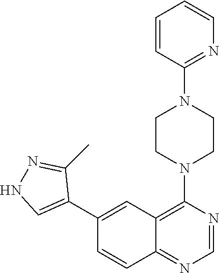 [SMILES: Cc1n[nH]cc1-c1ccc2ncnc(N3CCN(c4ccccn4)CC3)c2c1]

Compound 60 was prepared according to the same procedure to synthesize compound 50. 6-(3-methyl-1H-pyrazol-4-yl)-4-(4-(pyridin-2-yl)piperazin-1-yl)quinazoline, 60, (61 mg, 165 μmol, 41% yield) as a tan solid. LCMS [M+H]⁺ 372. 1H NMR (500 MHz, DMSO) δ12.80 (s, 1H), 8.62 (s, 1H), 8.15 (dd, J=5.0, 2.0 Hz, 1H), 7.99 (d, J=7.4 Hz, 3H), 7.83 (d, J=8.8 Hz, 1H), 7.57 (ddd, J=8.8, 7.1, 2.0 Hz, 1H), 6.86 (d, J=8.6 Hz, 1H), 6.67 (dd, J=7.1, 4.9 Hz, 1H), 3.87 (dd, J=7.0, 3.6 Hz, 4H), 3.75 (dd, J=6.9, 3.7 Hz, 4H), 2.50 (d, J=3.0 Hz, 3H).

### 4-(4-(4-(3-fluoropyridin-2-yl)piperazin-1-yl)quinazolin-6-yl)pyridin-2-amine, 61

Compound 61 was prepared via General Scheme 1 via the two step procedure reported for the preparation of compound 1. The second step is reported below.

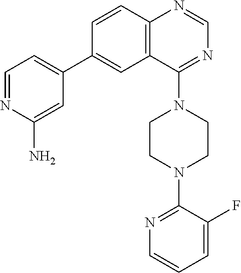 [SMILES: Nc1cc(-c2ccc3ncnc(N4CCN(c5ncccc5F)CC4)c3c2)ccn1]

To a microwave vial potassium phosphate, tribasic (328 mg, 3 eq, 1.548 mmol), PdCl₂(dppf) (37.7 mg, 0.1 Eq, 51.5 μmol), 4-(4,4,5,5-tetramethyl-1,3,2-dioxaborolan-2-yl)pyridin-2-amine (124 mg, 1.1 eq, 567 μmol), and 6-bromo-4-(4-(3-fluoropyridin-2-yl)piperazin-1-yl)quinazoline (200 mg, 1 eq, 515 μmol) was added and the vial was sealed. The solids were dissolved in 1,4-Dioxane (2.0 mL) and water (0.40 mL), and the reaction mixture was heated to 90° C. where it stirred for 16 hours. After time elapsed, the reaction mixture was cooled to room temperature, diluted with ethyl acetate, and filtered through a thick celite pad. The filtrate was collected and concentrated via rotary evaporation. The resulting residue was dissolved in 50 mL of ethyl acetate and washed with water (3×50 mL). The organic layer was dried over sodium sulfate, filtered, and concentrated via rotary evaporation. The crude concentrate was purified via column chromatography (0-10% MeOH/DCM) producing 4-(4-(4-(3-fluoropyridin-2-yl)piperazin-1-yl)quinazolin-6-yl)pyridin-2-amine, 61, (141 mg, 352.0 μmol, 68%). LCMS [M+H]⁺ 402. ¹H NMR (500 MHz, CDCl₃) δ 8.73 (s, 1H), 8.13 (d, J=5.4 Hz, 1H), 8.06 (d, J=2.0 Hz, 1H), 8.00 (dd, J=4.8, 1.7 Hz, 1H), 7.95-7.85 (m, 2H), 7.27-7.19 (m, 1H), 6.89-6.84 (m, 1H), 6.77 (ddd, J=7.9, 4.8, 3.1 Hz, 1H), 6.71 (s, 1H), 3.95-3.89 (m, 4H), 3.71-3.65 (m, 4H).

### 1-(1-(6-(2-aminopyridin-4-yl)quinazolin-4-yl)piperidin-4-yl)pyridin-2(1H)-one, 62

Compound 62 was prepared via General Scheme 1 via the two step procedure reported for the preparation of compound 1. The second step is reported below.

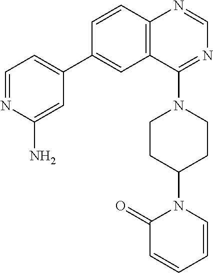 [SMILES: Nc1cc(-c2ccc3ncnc(N4CCC(n5ccccc5=O)CC4)c3c2)ccn1]

To a microwave vial potassium phosphate, tribasic (306 mg, 3 eq, 1.44 mmol), PdCl₂(dppf) (35 mg, 0.1 eq, 48.0 μmol), 4-(4,4,5,5-tetramethyl-1,3,2-dioxaborolan-2-yl)pyridin-2-amine (111 mg, 1.05 eq, 504 μmol), and 1-(1-(6-bromoquinazolin-4-yl)piperidin-4-yl)pyridin-2(1H)-one (185 mg, 1 eq, 480 μmol) was added and the vial was sealed. The solids were dissolved in 1,4-Dioxane (2.0 mL) and water (0.40 mL), and the reaction mixture was heated to 90° C. where it stirred for 16 hours. After time elapsed, the reaction mixture was cooled to room temperature, diluted with ethyl acetate, and filtered through a thick celite pad. The filtrate was collected and concentrated via rotary evaporation. The resulting residue was dissolved in 50 mL of ethyl acetate and washed with water (3×50 mL). The organic layer was dried over sodium sulfate, filtered, and concentrated via rotary evaporation. The crude concentrate was purified via column chromatography (0-10% MeOH/DCM) producing 1-(1-(6-(2-aminopyridin-4-yl)quinazolin-4-yl)piperidin-4-yl)pyridin-2(1H)-one, 62, (142 mg, 358 μmol, 74%). LCMS [M+H]⁺ 402. ¹H NMR (500 MHz, CDCl₃) δ 8.69 (s, 1H), 8.13-8.07 (m, 2H), 8.03-7.97 (m, 1H), 7.91-7.82 (m, 2H), 7.56-7.49 (m, 1H), 6.87-6.78 (m, 2H), 6.72-6.66 (m, 2H), 5.36 (tt, J=7.8, 3.8 Hz, 1H), 4.08 (ddd, J=11.9, 7.2, 3.6 Hz, 2H), 3.68 (ddd, J=12.9, 8.4, 3.3 Hz, 2H), 2.19 (ddt, J=14.4, 7.5, 3.6 Hz, 2H), 1.98 (dtd, J=12.2, 8.0, 3.4 Hz, 2H).

### 4-(4-(4-(2,4-difluorophenyl)piperidin-1-yl)quinazolin-6-yl)pyridin-2-amine, 63

Compound 63 was prepared via General Scheme 1 via the two step procedure reported for the preparation of compound 1. The two steps are reported below.

### 6-bromo-4-(4-(2,4-difluorophenyl)piperidin-1-yl)quinazoline, 136

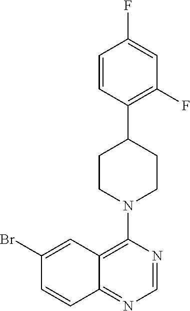 [SMILES: Fc1ccc(C2CCN(c3ncnc4ccc(Br)cc34)CC2)c(F)c1]

A solution of 6-bromo-4-chloroquinazoline (74 mg, 1.2 eq, 0.30 mmol) and 4-(2,4-difluorophenyl)piperidine (50 mg, 1.0 eq, 0.25 mmol) in NMP (0.51 mL, 0.4 M) was added triethylamine (64 mg, 88 L, 2.5 eq, 0.63 mmol). The mixture was stirred at 80° C. for 2 h. Upon cooling to room temperature, the reaction was extracted with EtOAc, washed with brine (×3) and water (×3), dried over anhydrous sodium sulfate, filtered, concentrated, and dry-loaded onto silica gel and purified on a 12 g silica gel column (0-10% EtOAc/Hexanes) to afford 6-bromo-4-(4-(2,4-difluorophenyl)piperidin-1-yl)quinazoline, 136, (78 mg, 0.19 mmol, 76%). LCMS [M+H]⁺ 405. 1H NMR (400 MHz, CDCl3) δ 8.73 (s, 1H), 8.05 (d, J=2.0 Hz, 1H), 7.90 (d, J=9.1 Hz, 1H), 7.83 (dd, J=8.9, 2.1 Hz, 1H), 7.18 (tt, J=8.3, 6.3 Hz, 1H), 6.88 (t, J=8.5 Hz, 2H), 4.57 (d, J=13.1 Hz, 2H), 3.39 (tt, J 12.4, 3.8 Hz, 1H), 3.29 (t, J=12.8 Hz, 2H), 2.36 (qd, J=12.9, 3.8 Hz, 2H), 2.02-1.88 (m, 2H).

### 4-(4-(4-(2,4-difluorophenyl)piperidin-1-yl)quinazolin-6-yl)pyridin-2-amine, 63

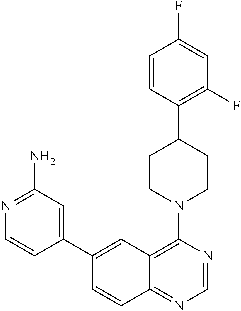 [SMILES: Nc1cc(-c2ccc3ncnc(N4CCC(c5ccc(F)cc5F)CC4)c3c2)ccn1]

In a 5 mL microwave vial, 6-bromo-4-(4-(2,4-difluorophenyl)piperidin-1-yl)quinazoline (78 mg, 1 eq, 0.19 mmol), 4-(4,4,5,5-tetramethyl-1,3,2-dioxaborolan-2-yl)pyridin-2-amine (51 mg, 1.2 eq, 0.23 mmol), tripotassium phosphate (160 mg, 4 eq, 0.77 mmol), and [1,1′-Bis(diphenylphosphino)ferrocene]dichloropalladium(II) (11 mg, 0.08 eq, 15 μmol) were added. The mixture was capped, purged with argon, then injected with degassed 4:1 dioxane:H₂O (1.0 mL, 0.2 M), and was heated at 80° C. for 18 h. After this time, the mixture was cooled to room temperature, diluted with ethyl acetate (30 mL), and washed with water (10 mL×2). The aqueous layer was washed with ethyl acetate (20 mL×2). The combined organic layer was washed with brine (20 mL), dried over sodium sulfate, and evaporated in vacuo to obtain the crude product. The crude product was dry-loaded onto silica gel and purified on a 12 g silica gel column (0-3% MeOH/DCM) to 4-(4-(4-(2,4-difluorophenyl)piperidin-1-yl)quinazolin-6-yl)pyridin-2-amine, 63, (20 mg, 0.19 mmol, 25%) as a brown solid. LCMS [M+H]⁺ 418. 1H NMR (500 MHz, CDCl3) δ 8.75 (s, 1H), 8.18-8.05 (m, 2H), 8.00-7.88 (m, 2H), 7.17 (tt, J=8.3, 6.3 Hz, 1H), 6.92 (d, J=5.1 Hz, 1H), 6.87 (t, J=8.5 Hz, 2H), 6.81 (s, 1H), 4.98 (s, 2H), 4.55 (d, J=13.3 Hz, 2H), 3.37 (ddt, J 12.4, 7.6, 3.8 Hz, 1H), 3.28 (t, J=12.8 Hz, 2H), 2.45-2.29 (m, 2H), 1.96-1.85 (m, 2H). 13C NMR (126 MHz, DMSO) δ 163.78, 161.83, 161.75, 160.51, 159.88, 159.80, 154.08, 151.53, 148.60, 147.30, 135.20, 131.05, 128.84, 128.77, 128.68, 122.75, 119.80, 119.66, 119.51, 115.84, 112.19, 111.98, 109.98, 105.38, 50.01, 32.57, 29.88.

### 4-(4-(8-(pyridin-2-yl)-2,8-diazaspiro[4.5]decan-2-yl)quinazolin-6-yl)pyridin-2-amine, 64

Compound 64 was prepared via General Scheme 1 via the two step procedure reported for the preparation of compound 1. However this compound requires an additional step to add a pyridyl group. This step is shown below.

### 4-(4-(8-(pyridin-2-yl)-2,8-diazaspiro[4.5]decan-2-yl)quinazolin-6-yl)pyridin-2-amine, 64. 2-(6-bromoquinazolin-4-yl)-2,8-diazaspiro[4.5]decane, 137

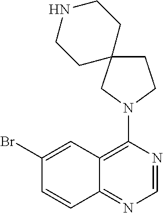 [SMILES: Brc1ccc2ncnc(N3CCC4(CCNCC4)C3)c2c1]

Prepared following similar procedure that was used to synthesize compound 117. tert-butyl 2-(6-bromoquinazolin-4-yl)-2,8-diazaspiro[4.5]decane-8-carboxylate, 137, (398 mg, 832 μmol, 100%) LCMS [M+H]⁺ 448. 1H NMR (400 MHz, CDCl3) δ 8.59 (s, 1H), 8.27-8.21 (m, 1H), 7.80-7.72 (m, 2H), 4.05 (t, J=7.0 Hz, 2H), 3.47 (td, J=6.3, 4.5 Hz, 4H), 3.41-3.31 (m, 1H), 2.42-2.32 (m, 1H), 2.02-1.94 (m, 2H), 1.62 (q, J=5.5 Hz, 4H), 1.44 (d, J=0.9 Hz, 9H). tert-butyl 2-(6-bromoquinazolin-4-yl)-2,8-diazaspiro[4.5]decane-8-carboxylate was then subjected to TFA in DCM (1:4, 7.8 mL) and let stir at room temperature for 3 h. Excess TFA was evaporated in vacuo to afford 2-(6-bromoquinazolin-4-yl)-2,8-diazaspiro[4.5]decane as a TFA salt (376 mg, 815 μmol, 98%). LCMS [M+H]⁺ 348. This compound was used without any further purification or characterization.

### 2-(6-bromoquinazolin-4-yl)-8-(pyridin-2-yl)-2,8-diazaspiro[4.5]decane, 138

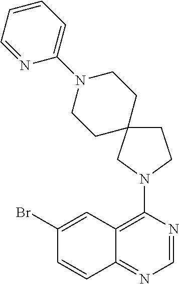 [SMILES: Brc1ccc2ncnc(N3CCC4(CCN(c5ccccn5)CC4)C3)c2c1]

To a 5 mL Biotage microwave vial was added 2-(6-bromoquinazolin-4-yl)-2,8-diazaspiro[4.5]decane, Trifluoroacetic acid (376 mg, 1.0 eq, 815 μmol) and potassium carbonate (338 mg, 3.0 eq, 2.45 mmol). The vial was capped and 2-fluoropyridine (237 mg, 3.0 eq, 2.45 mmol) and DMSO (1.63 mL, 0.5 M) were injected. The reaction was heated at 140° C. for 5 h. Upon completion, the reaction was extracted in EtOAc, washed with brine (×3) and water (×3), dried over sodium sulfate, filtered, concentrated in vacuo, and dry-loaded onto silica gel and purified on a 12 g silica gel column (0-20% EtOAc:Hexanes) to afford 2-(6-bromoquinazolin-4-yl)-8-(pyridin-2-yl)-2,8-diazaspiro[4.5]decane, 138, (144 mg, 339 μmol, 70%). LCMS [M+H]⁺ 425. 1H NMR (500 MHz, CDCl3) δ 8.39 (s, 1H), 8.04 (d, J=2.1 Hz, 1H), 7.98 (ddd, J=5.0, 2.0, 0.8 Hz, 1H), 7.54 (dd, J=8.9, 2.0 Hz, 1H), 7.49 (d, J=8.9 Hz, 1H), 7.26 (ddd, J=8.9, 7.1, 2.0 Hz, 1H), 6.46 (dd, J=8.6, 1.0 Hz, 1H), 6.40 (ddd, J=7.2, 4.9, 0.9 Hz, 1H), 3.84 (t, J=7.0 Hz, 2H), 3.59 (s, 2H), 3.41 (dtdd, J=17.7, 13.3, 9.1, 4.4 Hz, 4H), 1.80 (t, J=7.0 Hz, 2H), 1.64-1.47 (m, 4H).

### 4-(4-(8-(pyridin-2-yl)-2,8-diazaspiro[4.5]decan-2-yl)quinazolin-6-yl)pyridin-2-amine, 64

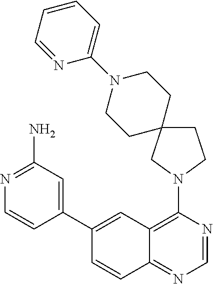 [SMILES: Nc1cc(-c2ccc3ncnc(N4CCC5(CCN(c6ccccn6)CC5)C4)c3c2)ccn1]

Compound 64 was prepared according to the same procedure to synthesize compound 50. 4-(4-(8-(pyridin-2-yl)-2,8-diazaspiro[4.5]decan-2-yl)quinazolin-6-yl)pyridin-2-amine, 64, (76 mg, 173 μmol, 51% yield) as a brown solid. LCMS [M+H]⁺ 438. 1H NMR (500 MHz, CDCl3) δ 8.63 (s, 1H), 8.33 (d, J=1.3 Hz, 1H), 8.22-8.15 (m, 2H), 7.90 (d, J=1.3 Hz, 2H), 7.47 (ddd, J=8.9, 7.1, 2.0 Hz, 1H), 6.90 (dd, J=5.4, 1.6 Hz, 1H), 6.75-6.70 (m, 1H), 6.68 (dd, J=8.6, 1.2 Hz, 1H), 6.61 (ddd, J=7.2, 4.9, 0.9 Hz, 1H), 4.57 (s, 2H), 4.14 (t, J=7.0 Hz, 2H), 3.87 (s, 2H), 3.72-3.53 (m, 4H), 2.04 (d, J=14.0 Hz, 2H), 1.82-1.74 (m, 4H). 13C NMR (126 MHz, CDCl3) δ 160.25, 159.47, 159.18, 155.07, 151.97, 149.86, 149.15, 148.17, 137.67, 135.13, 130.92, 129.11, 123.81, 116.64, 113.29, 112.79, 107.39, 106.28, 60.74, 49.68, 43.03, 41.18, 36.01, 34.15.

### 4-(4-(2-(pyridin-2-yl)-2,8-diazaspiro[4.5]decan-8-yl)quinazolin-6-yl)pyridin-2-amine, 65

Compound 65 was prepared via General Scheme 1 via the two step procedure reported for the preparation of compound 1. The two steps are reported below.

### 8-(6-bromoquinazolin-4-yl)-2-(pyridin-2-yl)-2,8-diazaspiro[4.5]decane, 139

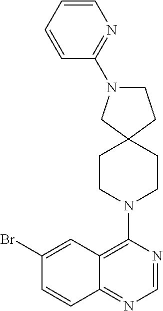 [SMILES: Brc1ccc2ncnc(N3CCC4(CCN(c5ccccn5)C4)CC3)c2c1]

Prepared using same procedure as seen for compound 131. 8-(6-bromoquinazolin-4-yl)-2-(pyridin-2-yl)-2,8-diazaspiro[4.5]decane, 139, (263 mg, 620 μmol, 44%). LCMS [M+H]⁺ 425. 1H NMR (500 MHz, CDCl3) δ 8.53 (s, 1H), 7.98 (dd, J=5.3, 1.9 Hz, 1H), 7.81 (d, J=2.0 Hz, 1H), 7.62-7.52 (m, 2H), 7.26 (ddd, J=8.8, 7.1, 2.0 Hz, 1H), 6.43-6.31 (m, 1H), 6.17 (d, J=8.5 Hz, 1H), 3.76-3.63 (m, 2H), 3.50 (ddd, J 13.1, 7.4, 4.7 Hz, 2H), 3.36 (t, J=7.0 Hz, 2H), 3.31 (s, 2H), 1.82 (q, J=6.9 Hz, 2H), 1.71-1.57 (m, 4H).

### 4-(4-(2-(pyridin-2-yl)-2,8-diazaspiro[4.5]decan-8-yl)quinazolin-6-yl)pyridin-2-amine, 65

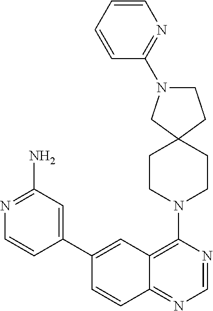 [SMILES: Nc1cc(-c2ccc3ncnc(N4CCC5(CCN(c6ccccn6)C5)CC4)c3c2)ccn1]

Compound 65 was prepared according to the same procedure to synthesize compound 50. 4-(4-(2-(pyridin-2-yl)-2,8-diazaspiro[4.5]decan-8-yl)quinazolin-6-yl)pyridin-2-amine, 65, (79 mg, 181 μmol, 29% yield) as a brown solid. LCMS [M+H]⁺ 438. 1H NMR (500 MHz, CDCl3) δ 8.74 (s, 1H), 8.21-8.13 (m, 2H), 8.05 (d, J=1.9 Hz, 1H), 7.99-7.89 (m, 2H), 7.45 (ddd, J=8.8, 7.1, 1.9 Hz, 1H), 6.90 (dd, J=5.4, 1.5 Hz, 1H), 6.73 (t, J=1.0 Hz, 1H), 6.55 (ddd, J=7.1, 5.1, 1.0 Hz, 1H), 6.36 (dt, J=8.6, 1.0 Hz, 1H), 4.61 (s, 2H), 3.95 (dt, J=13.5, 5.2 Hz, 2H), 3.75 (ddd, J=13.0, 7.4, 4.8 Hz, 2H), 3.55 (t, J=7.0 Hz, 2H), 3.51 (s, 2H), 2.01 (t, J=7.0 Hz, 2H), 1.91-1.81 (m, 4H). 13C NMR (126 MHz, CDCl3) δ 165.00, 159.22, 157.40, 154.73, 152.18, 149.44, 149.18, 148.36, 137.22, 135.78, 131.27, 129.50, 123.38, 116.82, 112.71, 111.71, 106.47, 106.29, 56.55, 47.83, 45.17, 41.16, 36.65, 35.20.

### 4-(4-((3aR,6aS)-5-(pyridin-2-yl)hexahydropyrrolo[3,4-c]pyrrol-2(1H)-yl)quinazolin-6-yl)pyridin-2-amine, 66

Compound 66 was prepared via General Scheme 1 via the two step procedure reported for the preparation of compound 1. The second step is reported below.

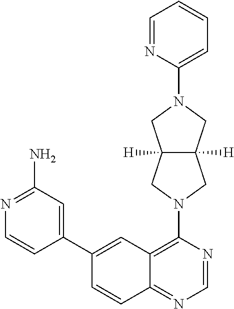 [SMILES: Nc1cc(-c2ccc3ncnc(N4C[C@H]5CN(c6ccccn6)C[C@H]5C4)c3c2)ccn1]

Compound 66 was prepared according to the same procedure to synthesize compound 50. 4-(4-((3aR,6aS)-5-(pyridin-2-yl)hexahydropyrrolo[3,4-c]pyrrol-2(1H)-yl)quinazolin-6-yl)pyridin-2-amine, 66, (14 mg, 34 μmol, 13% yield) as a brown solid. LCMS [M+H]⁺ 410. 1H NMR (500 MHz, DMSO) δ 8.50-8.41 (m, 2H), 8.06 (dd, J=5.3, 1.9 Hz, 1H), 8.03-7.94 (m, 2H), 7.79 (d, J=8.6 Hz, 1H), 7.48 (ddd, J=8.8, 7.1, 2.0 Hz, 1H), 6.91 (dd, J=5.3, 1.7 Hz, 1H), 6.80 (d, J=1.7 Hz, 1H), 6.55 (dd, J=7.1, 5.0 Hz, 1H), 6.43 (d, J=8.5 Hz, 1H), 6.01 (s, 2H), 4.30 (dd, J=11.7, 6.5 Hz, 2H), 3.95 (dd, J=11.7, 3.9 Hz, 2H), 3.69 (dd, J=10.7, 6.8 Hz, 2H), 3.43 (dd, J=10.9, 3.8 Hz, 2H), 3.18 (t, J=5.3 Hz, 2H). 13C NMR (126 MHz, DMSO) δ 160.46, 159.18, 156.95, 154.38, 151.25, 148.63, 147.82, 147.52, 136.95, 134.37, 130.41, 128.16, 123.68, 115.92, 111.42, 110.14, 106.53, 105.13, 54.38, 50.23.

### 4-(6-(2-aminopyridin-4-yl)pyrido[3,2-d]pyrimidin-4-yl)-1-(2-fluorophenyl)piperazin-2-one, 67

Compound 67 was prepared via General Scheme 3 via the five step procedure as reported for compound 32.

### tert-butyl 4-(2-fluorophenyl)-3-oxopiperazine-1-carboxylate, 140

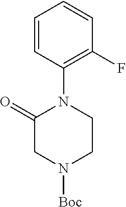 [SMILES: CC(C)(C)OC(=O)N1CCN(c2ccccc2F)C(=O)C1]

Prepared in an analogous manner as Step 2, General Scheme 3 as seen for compound 32. tert-butyl 4-(2-fluorophenyl)-3-oxopiperazine-1-carboxylate, 140, (539 mg, 1.83 mmol, 100%). LCMS [M+H]⁺ 295. 1H NMR (500 MHz, CDCl3) δ 7.35-7.27 (m, 2H), 7.23-7.14 (m, 2H), 4.27 (s, 2H), 3.80 (t, J=5.3 Hz, 2H), 3.68 (t, J=5.3 Hz, 2H), 1.50 (s, 9H).

### 1-(2-fluorophenyl)piperazin-2-one, 141

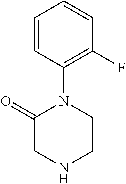 [SMILES: O=C1CNCCN1c1ccccc1F]

Prepared in an analogous manner as Step 3, General Scheme 3 as seen for compound 32. 1-(2-fluorophenyl)piperazin-2-one, 141, (129 mg, 662 μmol, 34%). LCMS [M+H]⁺ 195. This compound was used without any further purification or characterization.

### 4-(6-chloropyrido[3,2-d]pyrimidin-4-yl)-1-(2-fluorophenyl)piperazin-2-one, 142

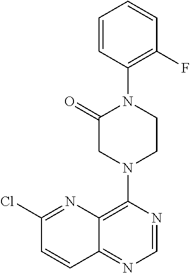 [SMILES: O=C1CN(c2ncnc3ccc(Cl)nc23)CCN1c1ccccc1F]

Prepared in an analogous manner as Step 4, General Scheme 3 as seen for compound 32. 4-(6-chloropyrido[3,2-d]pyrimidin-4-yl)-1-(2-fluorophenyl)piperazin-2-one, 142, (120 mg, 336 μmol, 60%). LCMS [M+H]⁺ 358. 1H NMR (500 MHz, DMSO) δ 8.27 (s, 1H), 7.86 (d, J=8.9 Hz, 1H), 7.55 (d, J=8.8 Hz, 1H), 7.10 (td, J=7.8, 1.7 Hz, 1H), 7.06-6.98 (m, 1H), 6.95 (ddd, J=10.0, 8.3, 1.5 Hz, 1H), 6.89 (td, J=7.6, 1.5 Hz, 1H), 4.70 (s, 2H), 4.27 (s, 2H), 3.49 (t, J=5.3 Hz, 2H).

### 4-(6-(2-aminopyridin-4-yl)pyrido[3,2-d]pyrimidin-4-yl)-1-(2-fluorophenyl)piperazin-2-one, 67

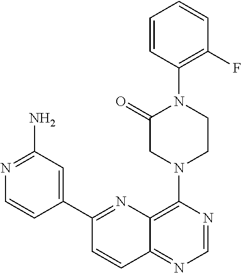 [SMILES: Nc1cc(-c2ccc3ncnc(N4CCN(c5ccccc5F)C(=O)C4)c3n2)ccn1]

Prepared in an analogous manner as Step 4, General Scheme 3 as seen for compound 32. 4-(6-(2-aminopyridin-4-yl)pyrido[3,2-d]pyrimidin-4-yl)-1-(2-fluorophenyl)piperazin-2-one, 67, (34 mg, 83 μmol, 42%) as a tan solid. LCMS [M+H]⁺ 416. 1H NMR (500 MHz, DMSO) δ 8.64 (s, 1H), 8.35 (d, J=8.8 Hz, 1H), 8.28 (d, J=8.8 Hz, 1H), 8.07 (d, J=5.3 Hz, 1H), 7.50 (td, J=7.7, 1.7 Hz, 1H), 7.43-7.38 (m, 1H), 7.34 (ddd, J=10.1, 8.3, 1.5 Hz, 1H), 7.28 (td, J=7.6, 1.5 Hz, 1H), 7.21 (dd, J=5.4, 1.6 Hz, 1H), 7.16 (s, 1H), 6.18 (s, 2H), 5.01 (s, 4H), 3.98 (s, 2H).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A compound encompassed within one of the following formulas: 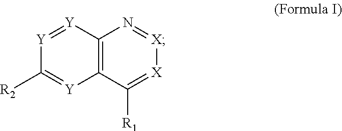
including pharmaceutically acceptable salts, solvates, and/or prodrugs thereof,
wherein each of X, Y, R1 and R2 independently include any chemical moiety that permits the resulting compound to inhibit DYRK1A, DYRK1B, DYRK2, DYRK3, CLK1, CLK2, CLK3, CLK4, homeodomain-interacting kinases (HIPKs), and/or CMGC kinases leading to inhibition of WNT signaling.

2. The compound of claim 1, wherein each of X, Y, R1 and R2 independently include any chemical moiety that permits the resulting compound to inhibit one or more of DYRK1A activity;
DYRK1A related PI3K/Akt signaling;
DYRK1A related tau phosphorylation;
DYRK1A related NFAT phosphorylation;
DYRK1A related ASK1/JNK1 pathway activation;
DYRK1A related p53 phosphorylation;
DYRK1 A related Amph 1 phosphorylation;
DYRK1A related Dynamin 1 phosphorylation;
DYRK1A related Synaptojanin phosphorylation;
DYRK1A related presenilin 1 (the catalytic sub-unit of γ-secretase) activity;
DYRK1A related amyloid precursor protein phosphorylation;
DYRK1A related SIRT1 activation;
DYRK2 related heat shock factor 1 and 26S proteasome activities;
DYRK3 related mTOR activity;
DYRK3 phosphorylation (e.g., PRAS40);
DYRK1B activity;
CMGC/CLK kinase activity;
CLK1 activity;
CLK2 activity;
CLK3 activity;
CLK4 activity;
CDK7 activity;
CDK8 activity;
CDK19 activity;
CDK8/19 activity;
PI3K activity;
PI3K mutant activity;
PDGFrA/B activity;
mTOR activity;
c-KIT activity;
RYK activity; and
WNT signaling.

3. The compound of claim 1, wherein each of X, Y, R1 and R2 independently include any chemical moiety that permits the resulting compound capable of binding to a DYRK or CLK protein.

4. The compound of claim 1,
wherein one of the “X” substituents is carbon and the other is nitrogen, or wherein both of the “X” substituents are carbon; and
wherein one of the “Y” substituents is nitrogen and the other “Y” substituents are carbon, or wherein two of the “Y” substituents are nitrogen and one “Y” substituent is carbon, or wherein all of the “Y” substituents are carbon.

5. The compound of claim 4, wherein the resulting formula is selected from:

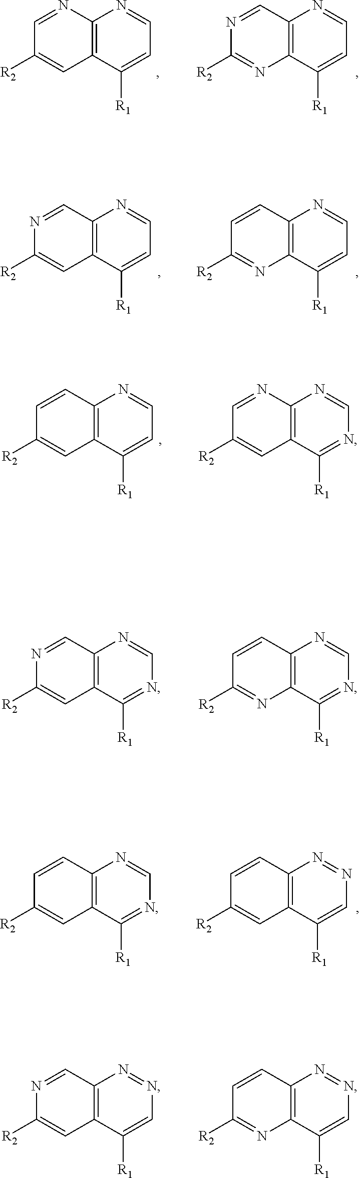

6. The compound of claim 1, wherein R1 is selected from hydrogen 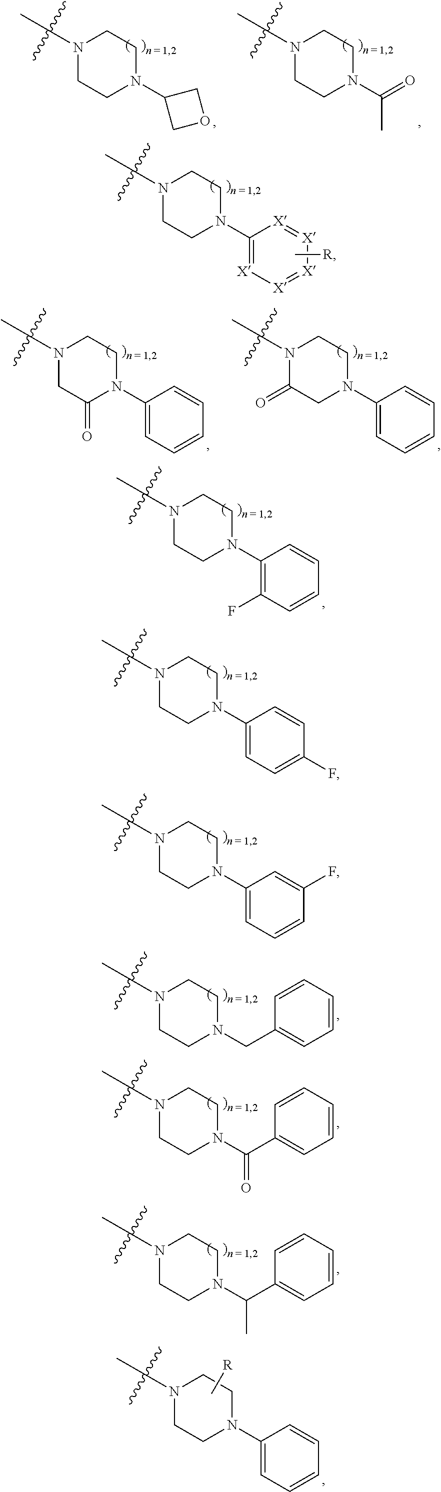 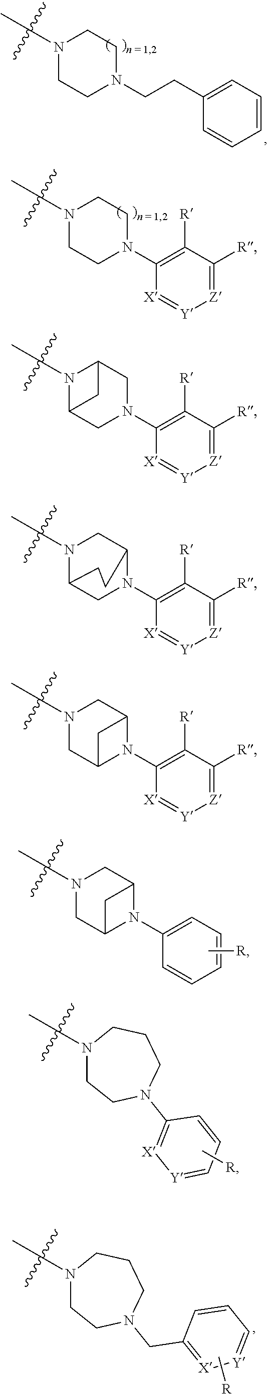 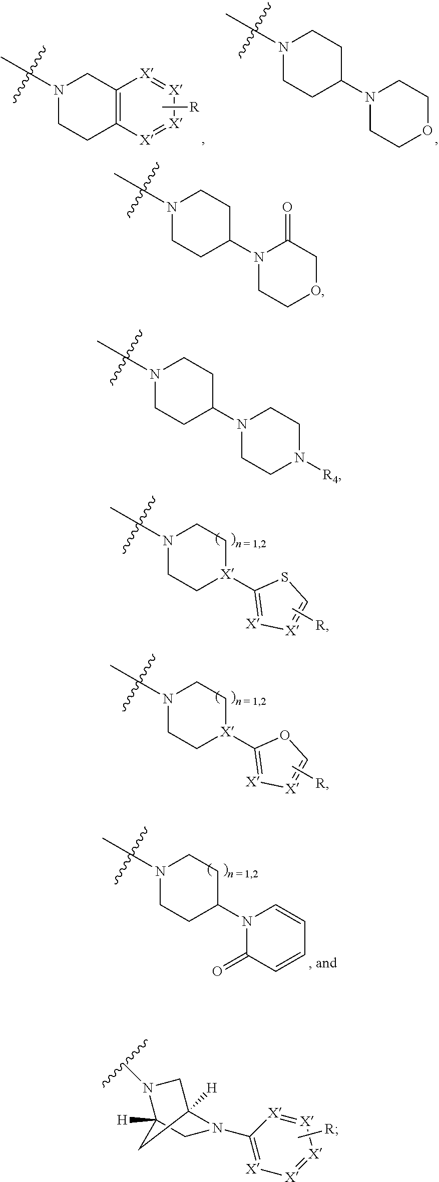
wherein R4 is selected from

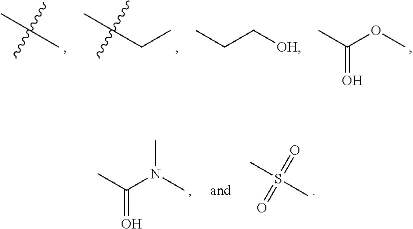

7. The compound of claim 1, wherein R2 is selected from hydrogen, halogen (e.g., fluorine, bromine, iodine, chlorine), aryl, substituted aryl, heteroaryl, substituted heteroaryl, 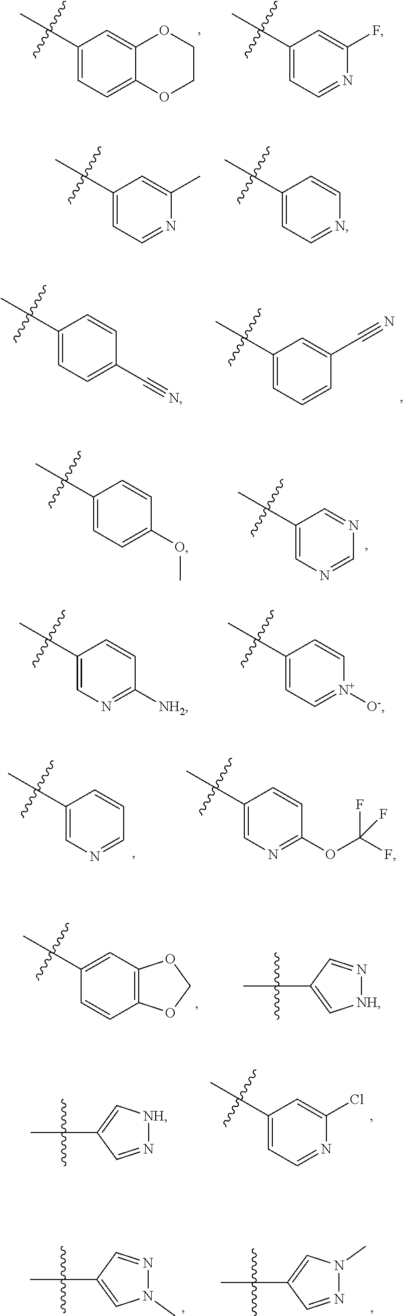 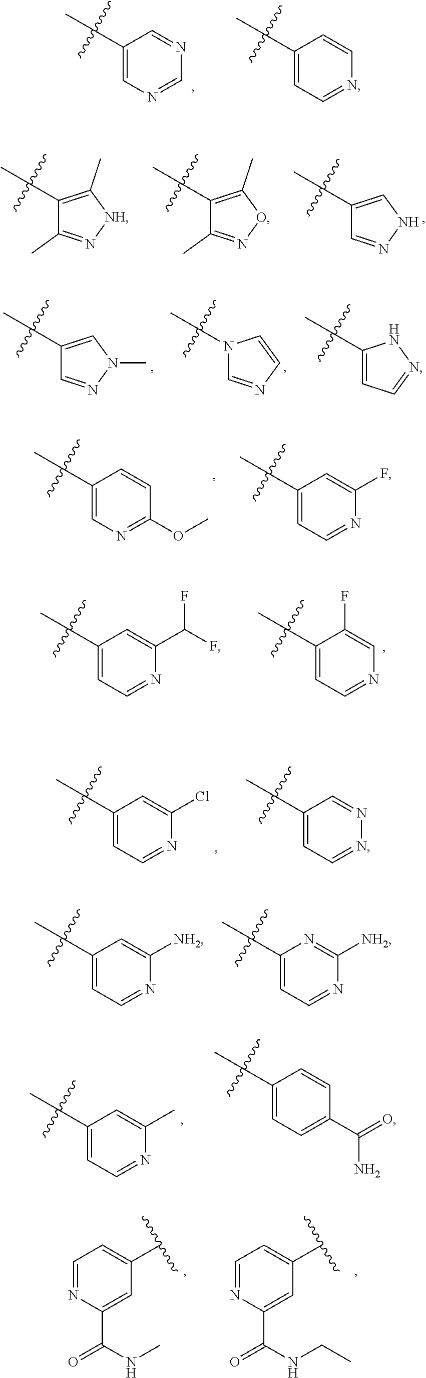 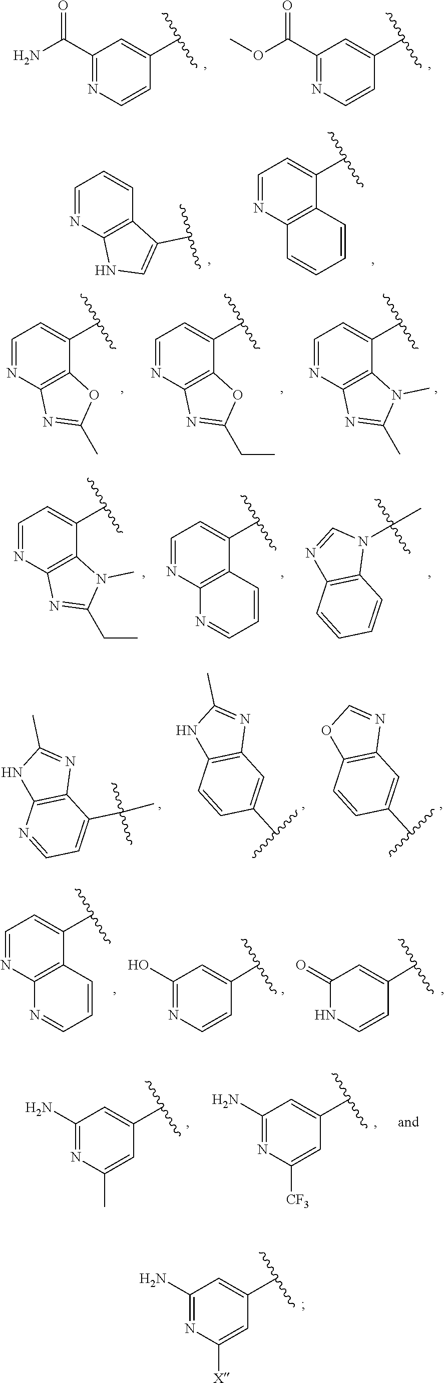
wherein X″ is selected from alkyl, haloalkyl, amino, alkylamino, hydroxy, fluoro, chloro, bromo, and cyano groups.

8. The compound of claim 6, wherein X′, Y′, and Z′ are independently N, C or CR′.

9. The compound of claim 6 or claim 7, wherein R, R′, and R″ are independently selected from hydrogen, halogen (e.g., fluorine, bromine, chlorine, iodine), di-halogen (di-fluorine, di-bromine, di-chlorine, di-iodine), CF3, OCH3, CHF2H, OCF3, methyl, di-methyl, alkoxy, alkylsulfonyl, cyano, carboxy, ester, amido, substituted amido, sulfonamide, substituted sulfonamide, methylenedioxy, heterocyclyl alkyl, heterocyclyl, heterocyclyl alkyl amido, a lipophilic moiety comprising ether.

10. The compound of claim 6 or claim 7, wherein R3 is selected from hydrogen, halogen (e.g., fluorine, bromine, chlorine, iodine), methyl, ethyl, and methoxy.

11. The compound of claim 1, wherein said compound is selected from the group of compounds recited in Table 1 and/or Compounds 1-67 recited in Example I.

12. A pharmaceutical composition comprising a compound of claim 1.

13. A method of treating, ameliorating, or preventing a disorder related to one or more of DYRK1A activity, DYRK1B activity, DYRK2 activity, DYRK3 activity, CLK1 activity, CLK2 activity, CLK3 activity, CLK4 activity, CDK7 activity, CDK8/19 activity, PI3K activity, PDGFrA/B activity, mTOR activity, WNT signaling activity, HIPK activity, and CMGC kinase activity leading to inhibition of WNT signaling, in a patient comprising administering to said patient a therapeutically effective amount of the pharmaceutical composition of claim 12.

14. The method of claim 13, wherein said disorder is selected from Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

15. The method of claim 13, wherein said patient is a human patient.

16. The method of claim 13, further comprising administering to said patient one or more agents for treating Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

17. A kit comprising a compound of claim 1 and instructions for administering said compound to a patient having a disorder related to one or more of DYRK1A activity, DYRK1B activity, DYRK2 activity, DYRK3 activity, CLK1 activity, CLK2 activity, CLK3 activity, CLK4 activity, CDK7 activity, CDK8/19 activity, PI3K activity, PDGFrA/B activity, mTOR activity, WNT signaling activity, HIPK activity, and CMGC kinase activity leading to inhibition of WNT signaling.

18. The kit of claim 17, wherein the disorder is Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

19. The kit of claim 17, further comprising one or more agents for treating Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.

20. A method for inhibiting one or more of DYRK1A activity, DYRK1B activity, DYRK2 activity, DYRK3 activity, CLK1 activity, CLK2 activity, CLK3 activity, CLK4 activity, CDK7 activity, CDK8/19 activity, PI3K activity, PDGFrA/B activity, mTOR activity, WNT signaling activity, HIPK activity, and CMGC kinase activity leading to inhibition of WNT signaling in a subject, comprising administering to the subject a compound of claim 1.

21. The method of claim 20, wherein administration of the compound results in inhibition of one or more of
DYRK1A activity;
DYRK1A related PI3K/Akt signaling;
DYRK1A related tau phosphorylation;
DYRK1A related NFAT phosphorylation;
DYRK1A related ASK1/JNK1 pathway activation;
DYRK1A related p53 phosphorylation;
DYRK1A related Amph 1 phosphorylation;
DYRK1A related Dynamin 1 phosphorylation;
DYRK1A related Synaptojanin phosphorylation;
DYRK1A related presenilin 1 (the catalytic sub-unit of γ-secretase) activity;
DYRK1A related amyloid precursor protein phosphorylation;
DYRK1A related SIRT1 activation;
DYRK2 related heat shock factor 1 and 26S proteasome activities;
DYRK3 related mTOR activity;
DYRK3 phosphorylation (e.g., PRAS40);
DYRK1B activity;
CMGC/CLK kinase activity;
CLK1 activity;
CLK2 activity;
CLK3 activity;
CLK4 activity;
CDK7 activity;
CDK8 activity;
CDK19 activity;
CDK8/19 activity;
PI3K activity;
PI3K mutant activity;
PDGFrA/B activity;
mTOR activity;
c-KIT activity;
RYK activity; and
WNT signaling.

22. The method of claim 20, wherein the subject is human subject suffering from or at risk for developing a disorder related to DYRK1A activity, DYRK1B activity, DYRK2 activity, DYRK3 activity, CLK1 activity, CLK2 activity, CLK3 activity, CLK4 activity, CDK7 activity, CDK8/19 activity, PI3K activity, PDGFrA/B activity, mTOR activity, WNT signaling activity, HIPK activity, and CMGC kinase activity leading to inhibition of WNT signaling.

23. The method of claim 20, wherein the disorder is Alzheimer's disease, down syndrome, diabetes, autoimmune diseases, inflammatory disorders (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer and metastatic colorectal cancer (e.g., metastatic colorectal cancer in the liver)), and other diseases.
